(19) United States
(12) Patent Application Publication
(10) Pub. No.: US 2006/0183800 A1
(43) Pub. Date: Aug. 17, 2006

(54) Methods and fluorinated compositions for treating amyloid-related diseases

(76) Inventors: Xianqi Kong, Dollard-des-Ormeaux (CA); Xinfu Wu, Laval (CA); Abderrahim Bouzide, Laval (CA); Isabelle Valade, Laval (CA); David Migneault, Laval (CA); Francesco Bellini, Town of Mount Royal (CA)

Correspondence Address: LAHIVE & COCKFIELD, 28 STATE STREET, BOSTON, MA, 02109, US

(21) Appl. No.: 11/274,761

(22) Filed: Nov. 14, 2005

Related U.S. Application Data
(60) Provisional application No. 60/627,765, filed on Nov. 12, 2004.
(60) Provisional application No. 60/638,965, filed on Dec. 22, 2004.

(51) Int. Cl. A61K 31/185; C07C 309/15

(52) U.S. Cl. 514/553; 562/104; 562/105

(57) ABSTRACT

Methods, compounds, pharmaceutical compositions and kits are described for treating or preventing amyloid-related disease. Also described are methods, compounds, pharmaceutical compositions and kits for detecting, diagnosing, monitoring and treating or preventing amyloid-related disease.

### RELATED APPLICATIONS

This application claims priority to U.S. Ser. No. 60/638,965, filed Dec. 22, 2004 and U.S. Ser. No. 60/627,765 filed Nov. 12, 2004. The entire contents of each of these applications are incorporated herein in their entirety.

### BACKGROUND

Amyloidosis refers to a pathological condition characterized by the presence of amyloid fibrils. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringment appearance in polarized light after staining. They also share common ultrastructural features and common X-ray diffraction and infrared spectra.

Amyloid-related diseases can either be restricted to one organ or spread to several organs. The first instance is referred to as “localized amyloidosis” while the second is referred to as “systemic amyloidosis.”

Some amyloid diseases can be idiopathic, but most of these diseases appear as a complication of a previously existing disorder. For example, primary amyloidosis (AL amyloid) can appear without any other pathology or can follow plasma cell dyscrasia or multiple myeloma.

Secondary amyloidosis is usually seen associated with chronic infection (such as tuberculosis) or chronic inflammation (such as rheumatoid arthritis). A familial form of secondary amyloidosis is also seen in other types of familial amyloidosis, e.g., Familial Mediterranean Fever (FMF). This familial type of amyloidosis is genetically inherited and is found in specific population groups. In both primary and secondary amyloidosis, deposits are found in several organs and are thus considered systemic amyloid diseases.

“Localized amyloidoses” are those that tend to involve a single organ system. Different amyloids are also characterized by the type of protein present in the deposit. For example, neurodegenerative diseases such as scrapie, bovine spongiform encephalitis, Creutzfeldt-Jakob disease, and the like are characterized by the appearance and accumulation of a protease-resistant form of a prion protein (referred to as AScr or PrP-27) in the central nervous system. Similarly, Alzheimer's disease, another neurodegenerative disorder, is characterized by neuritic plaques and neurofibrillary tangles. In this case, the amyloid plaques found in the parenchyma and the blood vessel is formed by the deposition of fibrillar Aβ amyloid protein. Other diseases such as adult-onset diabetes (type II diabetes) are characterized by the localized accumulation of amyloid fibrils in the pancreas.

Once these amyloids have formed, there is no known, widely accepted therapy or treatment which significantly dissolves amyloid deposits in situ, prevents further amyloid deposition or prevents the initiation of amyloid deposition.

Each amyloidogenic protein has the ability to undergo a conformational change and to organize into β-sheets and form insoluble fibrils which may be deposited extracellularly or intracellularly. Each amyloidogenic protein, although different in amino acid sequence, has the same property of forming fibrils and binding to other elements such as proteoglycan, amyloid P and complement component. Moreover, each amyloidogenic protein has amino acid sequences which, although different, show similarities such as regions with the ability to bind to the glycosaminoglycan (GAG) portion of proteoglycan (referred to as the GAG binding site) as well as other regions which promote β-sheet formation. Proteoglycans are macromolecules of various sizes and structures that are distributed almost everywhere in the body. They can be found in the intracellular compartment, on the surface of cells, and as part of the extracellular matrix. The basic structure of all proteoglycans is comprised of a core protein and at least one, but frequently more, negatively charged polysaccharide chains (GAGs) attached to the core protein. Many different GAGs have been discovered including chondroitin sulfate, dermatan sulfate, keratan sulfate, heparin, and hyaluronan.

In specific cases, amyloid fibrils, once deposited, can become toxic to the surrounding cells. For example, the Aβ fibrils organized as senile plaques have been shown to be associated with dead neuronal cells, dystrophic neurites, astrocytosis, and microgliosis in patients with Alzheimer's disease. When tested in vitro, oligomeric (soluble) as well as fibrillar Aβ peptide was shown to be capable of triggering an activation process of microglia (brain macrophages), which would explain the presence of microgliosis and brain inflammation found in the brain of patients with Alzheimer's disease. Both oligomeric and fibrillar Aβ peptide can also induce neuronal cell death in vitro. See, e.g., M P Lambert, et al., Proc. Natl. Acad. Sci. USA 95, 6448-53 (1998).

In another type of amyloidosis seen in patients with type II diabetes, the amyloidogenic protein IAPP, when organized in oligomeric forms or in fibrils, has been shown to induce β-islet cell toxicity in vitro. Hence, appearance of IAPP fibrils in the pancreas of type II diabetic patients contributes to the loss of the β islet cells (Langerhans) and organ dysfunction which can lead to insulinemia.

Another type of amyloidosis is related to β₂ microglobulin and is found in long-term hemodialysis patients. Patients undergoing long term hemodialysis will develop β₂-microglobulin fibrils in the carpal tunnel and in the collagen rich tissues in several joints. This causes severe pains, joint stiffness and swelling.

Amyloidosis is also characteristic of Alzheimer's disease. Alzheimer's disease is a devastating disease of the brain that results in progressive memory loss leading to dementia, physical disability, and death over a relatively long period of time. With the aging populations in developed countries, the number of Alzheimer's patients is reaching epidemic proportions.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites) and activated microglia (microgliosis and astrocytosis). A main constituent of these amyloid plaques is the amyloid-β peptide (Aβ), a 39-43 amino-acid protein that is produced through cleavage of the β-amyloid precursor protein (APP). Extensive research has been conducted on the relevance of Aβ deposits in Alzheimer's disease, see, e.g., Selkoe, Trends in Cell Biology 8, 447-453 (1998). Aβ naturally arises from the metabolic processing of the amyloid precursor protein (“APP”) in the endoplasmic reticulum (“ER”), the Golgi apparatus, or the endosomal-lysosomal pathway, and most is normally secreted as a 40 (“Aβ1-40”) or 42 (“Aβ1-42”) amino acid peptide (Selkoe, Annu. Rev. Cell Biol. 10, 373-403 (1994)). A role for Aβ as a primary cause for Alzheimer's disease is supported by the presence of extracellular Aβ deposits in senile plaques of Alzheimer's disease, the increased production of Aβ in cells harboring mutant Alzheimer's disease associated genes, e.g., amyloid precursor protein, presenilin I and presenilin II; and the toxicity of extracellular soluble (oligomeric) or fibrillar Aβ to cells in culture. See, e.g., Gervais, Eur. Biopharm. Review, 40-42 (Autumn 2001); May, DDT 6, 459-62 (2001). Although symptomatic treatments exist for Alzheimer's disease, this disease cannot be prevented or cured at this time.

Alzheimer's disease is characterized by diffuse and neuritic plaques, cerebral angiopathy, and neurofibrillary tangles. Plaque and blood vessel amyloid is believed to be formed by the deposition of insoluble Aβ amyloid protein, which may be described as diffuse or fibrillary. Both soluble oligomeric Aβ and fibrillar Aβ are also believed to be neurotoxic and inflammatory.

Another type of amyloidosis is cerebral amyloid angiopathy (CAA). CAA is the specific deposition of amyloid-β fibrils in the walls of leptomingeal and cortical arteries, arterioles and veins. It is commonly associated with Alzheimer's disease, Down's syndrome and normal aging, as well as with a variety of familial conditions related to stroke or dementia (see Frangione et al., Amyloid: J. Protein Folding Disord. 8, Suppl. 1, 36-42 (2001)).

Presently available therapies for treatment of β-amyloid diseases are almost entirely symptomatic, providing only temporary or partial clinical benefit. Although some pharmaceutical agents have been described that offer partial symptomatic relief, no comprehensive pharmacological therapy is currently available for the prevention or treatment of, for example, Alzheimer's disease.

A variety of imaging techniques have been used to diagnose diseases. Included among these imaging techniques is X-ray imaging. In X-ray imaging, the images produced reflect the different densities of structures and tissue in the body of the patient. To improve the diagnostic usefulness of this imaging technique, contrast agents may be used to increase the density of tissues of interest relative to surrounding tissues. Examples of such contrast agents include, for example, barium and iodinated compounds, which may be used for X-ray studies of the gastrointestinal region, including the esophagus, stomach, intestines and rectum. Contrast agents may also be used for computed tomography (CT) and computer assisted tomography (CAT) studies to improve visualization of tissue of interest, for example, the gastrointestinal tract.

Magnetic resonance imaging (MRI) is another imaging technique. Unlike X-ray imaging, MRI does not involve ionizing radiation. MRI may be used for producing cross-sectional images of the body in a variety of scanning planes such as, for example, axial, coronal, sagittal or orthogonal. MRI employs a magnetic field, radio frequency energy and magnetic field gradients to make images of the body. The contrast or signal intensity differences between tissues mainly reflect the T1 (longitudinal) and T2 (transverse) relaxation values and the proton density, which generally corresponds to the free water content, of the tissues. To change the signal intensity in a region of a patient by the use of a contrast medium, several possible approaches are available. For example, a contrast medium may be designed to change the T1, the T2 or the proton density.

Generally speaking, MRI requires the use of contrast agents. If MRI is performed without employing a contrast agent, differentiation of the tissue of interest from the surrounding tissues in the resulting image may be difficult. In the past, attention has focused primarily on paramagnetic contrast agents for MRI. Paramagnetic contrast agents involve materials that contain unpaired electrons. The unpaired electrons act as small magnets within the main magnetic field to increase the rate of longitudinal (T1) and transverse (T2) relaxation. Paramagnetic contrast agents typically comprise metal ions, for example, transition metal ions, which provide a source of unpaired electrons. However, these metal ions are also generally highly toxic. In an effort to decrease toxicity, the metal ions are typically chelated with ligands.

Metal oxides, most notably iron oxides, have also been used as MRI contrast agents. While small particles (e.g., particles having a diameter of less than about 20 nm) of iron oxide may have desirable paramagnetic relaxation properties, their predominant effect is through bulk susceptibility. Nitroxides are another class of MRI contrast agents that are also paramagnetic. These have relatively low relaxivity and are generally less effective than paramagnetic ions.

These MRI contrast agents suffer from a number of limitations. For example, increased image noise may be associated with certain contrast agents, including contrast agents involving chelated metals. This noise generally arises out of intrinsic peristaltic motions and motions from respiration or cardiovascular action. In addition, the signal intensity for contrast agents generally depends upon the concentration of the agent as well as the pulse sequence used. Absorption of contrast agents can complicate interpretation of the images, particularly in the distal portion of the small intestine, unless sufficiently high concentrations of the paramagnetic species are used. See, e.g., Kormmesser et al., Magnetic Resonance Imaging, 6:124 (1988).

Other compounds which can be used for imaging include radiopharmaceuticals, which are drugs containing a radionuclide (e.g., ¹⁸F). Radiopharmaceuticals are used in the field of radiology known as nuclear medicine for the diagnosis or therapy of various diseases. In vivo diagnostic information may be obtained by administration, e.g., by intravenous injection, of a radiopharmaceutical and determining its biodistribution using a radiation-detecting camera. In PET, radio nuclides, typically fluorine-18, are incorporated into substances such to produce radiopharmaceuticals which are ingested by the patient. As the radio nuclides decay, positrons are emitted and they collide, in a very short distance, with an electron and become annihilated and converted into two photons, or gamma rays, traveling linearly in opposite directions to one another with each ray having an energy of 511 KeV. PET scanners typically include laterally spaced rings with detectors which encircle the patient. A typical detector within the ring is a BgO crystal in front of a photomultiplier tube. Each ring is thus able to discern an annihilation event occurring in a single plane. The analog PMT signals are analyzed by coincidence detection circuits to detect coincident or simultaneous signals generated by PMT's on opposite sides of the patient, i.e., opposed detectors on the ring. Specifically, when two opposed detectors detect simultaneous 511 KeV events, a line passing through both detectors establishes a line of response (LOR). By processing a number of LORs indicative of annihilation events an image is reconstructed of the organ using computed tomographic techniques.

Previously disclosed fluorine-containing imaging agents include: fluorinated fatty acid sulfonate derivates (U.S. Pat. No. 5,660,815); perfluoro-tert-butyl containing organic compounds (U.S. Pat. Nos. 5,116,599; 5,234,680; and 5,324,504); fluoro-substituted benzene derivatives (U.S. Pat. Nos. 5,130,119; 5,318,770; and 4,612,185); fluorine containing nitroxyl compounds (M. D. Adams et al., U.S. Pat. No. 5,362,477 issued 1994); fluorinated metal-chelating compounds and chelates (JP 6-136347, EP 592306, EP 603403, and JP 5-186372); fluorinated fullerenes (U.S. Pat. No. 5,248,498); fluorine-amine compounds (U.S. Pat. Nos. 4,960,815 and 5,081,304); N-methyl-glucamine salts (U.S. Pat. Nos. 4,639,364 and 4,913,853); fluorocarbons (WO 89/03693); perfluoro crown ethers (U.S. Pat. No. 4,838,274); perfluoro dioxolanes (U.S. Pat. No. 5,070,213); perfluoro tert-butyl aryl compounds (U.S. Pat. No. 5,401,493); ¹⁹F labeled dextrans and antibodies (U.S. Pat. No. 5,236,694); and perfluoro tert-butyl containing steroids (U.S. Pat. No. 5,397,563).

### SUMMARY OF THE INVENTION

The present invention relates to the use of certain fluorinated compounds in the treatment of amyloid-related diseases. In particular, the invention relates to a method of treating or preventing an amyloid-related disease in a subject comprising administering to the subject a therapeutic amount of a compound of the invention. The invention also pertains to each of the novel compounds of the invention as described herein. Among the compounds for use in the invention are those according to the following Formulae, such that, when administered, amyloid fibril formation, organ specific dysfunction (e.g., neurodegeneration), or cellular toxicity is reduced or inhibited.

Fluorine features a van der Waals radius (1.2 A) similar to hydrogen (1.35 A). Therefore, hydrogen replacement (with F) does not cause significant conformational changes. Fluorination can also lead to increased lipophilicity, thus enhancing the bioavailability of many drugs. The carbon-fluorine bond strength (460 kJ/mol in CH₃F) exceeds that of equivalent C—H bonds. Perfluorocarbons (PFCs) display high chemical and biological inertness and a capacity to dissolve considerable amounts of gases, particularly oxygen, carbon dioxide and air per unit volume. PFCs can dissolve about a 50% volume of oxygen at 37° C. under a pure oxygen atmosphere. Fluorocarbon formulations are useful in diagnostic procedures, for example as contrast agents (Riess, J. G., Hemocompatible Materials and Devices: Prospectives Towards the 21st Century, Technomics Publ. Co, Lancaster, Pa. USA, Chap 14 (1991); Vox Sanguinis, 61:225-239, 1991). Fluorocarbons are also believed to be safer and less toxic than other corresponding halogenated hydrocarbons, such as chlorocarbons. N-chlorinated compounds may decompose to form hydrochloric acid, which is toxic to subjects.

In one embodiment, the present invention pertains to fluorinated compounds of Formula I: 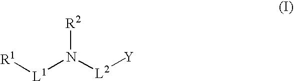
wherein:

R¹ is fluorine, hydrogen, a substituted or unsubstituted cycloalkyl, a substituted or unsubstituted aryl, a substituted or unsubstituted acyl, a substituted or unsubstituted arylcycloalkyl, a substituted or unsubstituted bicyclic or tricyclic ring, a bicyclic or tricyclic fused ring group, or a substituted or unsubstituted C₂-C₁₀ alkyl group;

R² is hydrogen, fluorine, a substituted or unsubstituted acyl, a substituted or unsubstituted alkyl, a substituted or unsubstituted mercaptoalkyl, a substituted or unsubstituted alkenyl, a substituted or unsubstituted alkynyl, a substituted or unsubstituted cycloalkyl, a substituted or unsubstituted aryl, a substituted or unsubstituted arylalkyl, a substituted or unsubstituted thiazolyl, a substituted or unsubstituted triazolyl, a substituted or unsubstituted imidazolyl, a substituted or unsubstituted benzothiazolyl, or a substituted or unsubstituted benzoimidazolyl;

Y is SO₃⁻X⁺, OSO₃⁻X⁺, SSO₃^{-X}⁺, or SO₂⁻X⁺;

X⁺ is hydrogen or a cationic group; and

L¹ and L² are each independently a substituted or unsubstituted C₁-C₁₂ alkyl group or absent;

and pharmaceutically acceptable salts, esters, or prodrugs thereof, provided that at least one of R¹, R², L¹, or L² comprise one or more fluorine atoms, provided that when L² comprises one fluorine atom and Y is SO₂⁻X⁺, at least one of R¹ and R² is not hydrogen.

In another embodiment, the compounds of formula (I) include the compounds of formula (II): 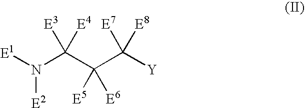
wherein:

E¹ and E² are each independently hydrogen or fluorine;

E³, E⁴, E⁵, E⁶, E⁷, and E⁸ are each independently fluorine, hydrogen, a substituted or unsubstituted cycloalkyl, a substituted or unsubstituted aryl, a substituted or unsubstituted acyl, a substituted or unsubstituted arylcycloalkyl, a substituted or unsubstituted bicyclic or tricyclic ring, a bicyclic or tricyclic fused ring group, or a substituted or unsubstituted C₂-C₁₀ alkyl group;

Y is SO₃⁻X⁺, OSO₃⁻X⁺, SSO₃⁻X⁺, or SO₂⁻X⁺;

X⁺ is hydrogen or a cationic group; and pharmaceutically acceptable salts, esters or prodrugs thereof, provided that at least one of E¹, E², E³, E⁴, E⁵, E⁶, E⁷, and E⁸ comprise one or more fluorine atoms.

In one embodiment, the compounds disclosed herein prevent or inhibit amyloid protein assembly into insoluble fibrils which, in vivo, are deposited in various organs, or they favor clearance of pre-formed deposits or slows deposition in patients already having deposits. In another embodiment, the compound may also prevent the amyloid protein, in its soluble, oligomeric form or in its fibrillar form, from binding or adhering to a cell surface and causing cell damage or toxicity. In another embodiment, the compounds may prevent formation of toxic oligomers and prevent oligomer induced toxicity. In yet another embodiment, the compound may block amyloid-induced cellular toxicity or macrophage activation. In another embodiment, the compound may block amyloid-induced neurotoxicity or microglial activation. In another embodiment, the compound protects cells from amyloid induced cytotoxicity of β-islet cells of the pancreas. In another embodiment, the compound may enhance clearance from a specific organ, e.g., the brain or it may decrease concentration of the amyloid protein in such a way that amyloid fibril formation is inhibited in the targeted organ.

The compounds of the invention may be administered therapeutically or prophylactically to treat diseases associated with amyloid fibril formation, aggregation or deposition. The compounds of the invention may act to ameliorate the course of an amyloid related disease using any of the following mechanisms (this list is meant to be illustrative and not limiting): slowing/preventing formation of toxic oligomers, slowing the rate of amyloid fibril formation or deposition; lessening the degree of amyloid deposition; inhibiting, reducing, or preventing amyloid fibril formation; inhibiting neurodegeneration or cellular toxicity induced by amyloid; inhibiting amyloid induced inflammation; enhancing the clearance of amyloid; or favoring the degradation of amyloid protein prior to its organization in oligomeric protofibrils or fibrils.

The compounds of the invention may be administered therapeutically or prophylactically to treat diseases associated with amyloid-β fibril formation, aggregation or deposition. The compounds of the invention may act to ameliorate the course of an amyloid-related disease using any of the following mechanisms (this list is meant to be illustrative and not limiting): slowing the rate of amyloid-β oligomerization, fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; enhancing the clearance of amyloid-β from the brain; or favoring the degradation of amyloid-β protein prior to its organization in fibrils.

Therapeutic compounds of the invention may be effective in controlling amyloid-β deposition either following their entry into the brain (following penetration of the blood brain barrier) or from the periphery. When acting from the periphery, a compound may alter the equilibrium of Aβ between the brain and the plasma so as to favor the exit of Aβ from the brain. It may also increase the catabolism of neuronal Aβ and change the rate of exit from the brain. An increase in the exit of Aβ from the brain would result in a decrease in Aβ brain and cerebral spinal fluid (CSF) concentration and therefore favor a decrease in Aβ deposition. Alternatively, compounds that penetrate the brain could control deposition by acting directly on brain Aβ e.g., by maintaining it in a non-oligomeric or non-fibrillar form, favoring its clearance from the brain, or by slowing down APP processing. These compounds could also prevent Aβ in the brain from interacting with the cell surface and therefore prevent neurotoxicity, neurodegeneration or inflammation. They may also decrease Aβ production by activated microglia. The compounds may also increase degradation by macrophages or neuronal cells.

In one embodiment, the method is used to treat Alzheimer's disease (e.g., sporadic, familial, or early AD). The method can also be used prophylactically or therapeutically to treat other clinical occurrences of amyloid-β deposition, such as in Down's syndrome individuals and in patients with cerebral amyloid angiopathy (“CAA”) or hereditary cerebral hemorrhage.

In another embodiment, the method is used to treat mild cognitive impairment. Mild Cognitive Impairment (“MCI”) is a condition characterized by a state of mild but measurable impairment in thinking skills, which is not necessarily associated with the presence of dementia. MCI frequently, but not necessarily, precedes Alzheimer's disease.

Additionally, abnormal accumulation of APP and of amyloid-β protein in muscle fibers has been implicated in the pathology of sporadic inclusion body myositis (IBM) (Askanas, et al., Proc. Natl. Acad. Sci. USA 93, 1314-1319 (1996); Askanas, et al., Current Opinion in Rheumatology 7, 486-496 (1995)). Accordingly, the compounds of the invention can be used prophylactically or therapeutically in the treatment of disorders in which amyloid-beta protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (AMD). AMD is a cause of irreversible vision loss in older individuals. It is believed that Aβ deposition could be an important component of the local inflammatory events that contribute to atrophy of the retinal pigmented epithelium, drusen biogenesis, and the pathogenesis of AMD (Johnson, et al., Proc. Natl. Acad. Sci. USA 99(18), 11830-5 (2002)).

The present invention therefore relates to the use of compounds of Formula I, or otherwise described herein in the prevention or treatment of amyloid-related diseases, including, inter alia, Alzheimer's disease, cerebral amyloid angiopathy, mild cognitive impairment, inclusion body myositis, Down's syndrome, macular degeneration, as well as other types of amyloidosis like IAPP-related amyloidosis (e.g., diabetes), primary (AL) amyloidosis, secondary (AA) amyloidosis and β₂ microglobulin-related (dialysis-related) amyloidosis.

In Type II diabetes related amyloidosis (IAPP), the amyloidogenic protein IAPP induces β-islet cell toxicity when organized in oligomeric forms or in fibrils. Hence, appearance of IAPP fibrils in the pancreas of type II diabetic patients contributes to the loss of the P islet cells (Langerhans) and organ dysfunction which leads to insulinemia.

Primary amyloidosis (AL amyloid) is usually found associated with plasma cell dyscrasia and multiple myeloma. It can also be found as an idiopathic disease.

Secondary (AA) amyloidosis is usually seen associated with chronic infection (such as tuberculosis) or chronic inflammation (such as rheumatoid arthritis). A familial form of secondary amyloidosis is also seen in Familial Mediterranean Fever (FMF).

β₂ microglobulin-related (dialysis-related) amyloidosis is found in long-term hemodialysis patients. Patients undergoing long term hemodialysis will develop β₂-microglobulin fibrils in the carpal tunnel and in the collagen rich tissues in several joints. This causes severe pains, joint stiffness and swelling. These deposits are due to the inability to maintain low levels of β₂M in plasma of dialyzed patients. Increased plasma concentrations of β₂M protein will induce structural changes and may lead to the deposition of modified β₂M as insoluble fibrils in the joints.

The fluorinated compounds of the invention also have numerous other applications as imaging probes, diagnostic reagents, and contrast agents.

### DETAILED DESCRIPTION OF THE INVENTION

The present invention relates to the use of compounds of Formula I, or compounds otherwise described herein in the treatment of amyloid-related diseases. For convenience, some definitions of terms referred to herein are set forth below.

Amyloid-Related Diseases

AA (Reactive) Amyloidosis

Generally, AA amyloidosis is a manifestation of a number of diseases that provoke a sustained acute phase response. Such diseases include chronic inflammatory disorders, chronic local or systemic microbial infections, and malignant neoplasms. The most common form of reactive or secondary (AA) amyloidosis is seen as the result of long-standing inflammatory conditions. For example, patients with Rheumatoid Arthritis or Familial Mediterranean Fever (which is a genetic disease) can develop AA amyloidosis. The terms “AA amyloidosis” and “secondary (AA) amyloidosis” are used interchangeably.

AA fibrils are generally composed of 8,000 Dalton fragments (AA peptide or protein) formed by proteolytic cleavage of serum amyloid A protein (ApoSAA), a circulating apolipoprotein which is mainly synthesized in hepatocytes in response to such cytokines as IL-1, IL-6 and TNF. Once secreted, ApoSAA is complexed with HDL. Deposition of AA fibrils can be widespread in the body, with a preference for parenchymal organs. The kidneys are usually a deposition site, and the liver and the spleen may also be affected. Deposition is also seen in the heart, gastrointestinal tract, and the skin.

Underlying diseases which can lead to the development of AA amyloidosis include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril amyloid deposits. These include such conditions as Hodgkin's lymphoma, renal carcinoma, carcinomas of gut, lung and urogenital tract, basal cell carcinoma, and hairy cell leukemia. Other underlying conditions that may be associated with AA amyloidosis are Castleman's disease and Schnitzler's syndrome.

AL Amyloidoses (Primary Amyloidosis)

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia. AL amyloidosis is also described in detail in Current Drug Targets, 2004, 5 159-171.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the kidney, liver, spleen and heart, may be involved.

Hereditary Systemic Amyloidoses

There are many forms of hereditary systemic amyloidoses. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins. Table 1 summarizes the fibril composition of exemplary forms of these disorders.

TABLE 1
Fibril Composition of Exemplary Amyloid-Related Diseases

| | Genetic | |
|---|---|---|
| Fibril Peptide/Protein | Variant | Clinical Syndrome |
| ATTR protein from Transthyretin | Met30, many | Familial amyloid polyneuropathy (FAP), |
| and fragments | others | (Mainly peripheral nerves) |
| ATTR protein from Transthyretin | Thr45, Ala60, | Cardiac involvement predominant without |
| and fragments | Ser84, Met111, | neuropathy, familial amyloid polyneuropathy, |
| | Ile122 | senile systemic amyloidosis, Tenosynovium |
| N-terminal fragment of | Arg26 | Familial amyloid polyneuropathy (FAP), |
| Apolipoprotein A1 (apoAI) | | (mainly peripheral nerves) |
| N-terminal fragment of | Arg26, Arg50, | Ostertag-type, non-neuropathic (predominantly |
| Apoliproprotein A1 (AapoAI) | Arg 60, others | visceral involvement) |
| AapoAII from Apolipoprotein AII | | Familial amyloidosis |
| Lysozyme (Alys) | Thr56, His67 | Ostertag-type, non-neuropathic (predominantly |
| | | visceral involvement) |
| Fibrogen alpha chain fragment | Leu554, Val | Cranial neuropathy with lattic corneal |
| | 526 | dystrophy |
| Gelsolin fragment (Agel) | Asn187, | Cranial neuropathy with lattice corneal |
| | Tyr187 | dystrophy |
| Cystatin C fragment (ACys) | Glu68 | Hereditary cerebral hemorrhage (cerebral |
| | | amyloid angiopathy) - Icelandic type |
| β-amyloid protein (Aβ) derived from | Gln693 | Hereditary cerebral hemorrhage (cerebral |
| Amyloid Precursor Protein (APP) | | amyloid angiopathy) - Dutch type |
| β-amyloid protein (Aβ) derived from | Ile717, Phe717, | Familial Alzheimer's Disease |
| Amyloid Precursor Protein (APP) | Gly717 | |
| β-amyloid protein (Aβ) derived from | Gln618 | Alzheimer's disease, Down's syndrome, |
| Amyloid Precursor Protein (APP), | | hereditary cerebral hemorrhage with |
| e.g., bPP 695 | | amyloidosis, Dutch type |
| β-amyloid protein (Aβ) derived from | Asn670, | Familial Dementia - probably Alzheimer's |
| Amyloid Precursor Protein (APP) | Leu671 | Disease |
| Prion Protein (PrP, APrP^{SC}) derived | Leu102, | Familial Creutzfeldt-Jakob disease; Gerstmann- |
| from Prp precursor protein (51-91 | Val167, | Sträussler-Scheinker syndrome (hereditary |
| insert) | Asn178, | spongiform encephalopathies, prion diseases) |
| | Lys200 | |
| AA derived from Serum amyloid A | | Familial Mediterranean fever, predominant |
| protein (ApoSAA) | | renal involvement (autosomal recessive) |
| AA derived from Serum amyloid A | | Muckle-Well's syndrome, nephropathy, |
| protein (ApoSAA) | | deafness, urticaria, limb pain |
| Unknown | | Cardiomyopathy with persistent atrial standstill |
| Unknown | | Cutaneous deposits (bullous, papular, |
| | | pustulodermal) |
| AH amyloid protein, derived from | Aγ I | Myeloma associated amyloidosis |
| immunoglobulin heavy chain | | |
| (gamma I) | | |
| ACal amyloid protein from | (Pro) calcitonin | Medullary carcinomas of the thyroid |
| (pro)calcitonin | | |
| AANF amyloid protein from atrial | | Isolated atrial amyloid |
| natriuretic factor | | |
| Apro from Prolactin | | Prolactinomas |
| Abri/ADan from ABri peptide | | British and Danish familial Dementia |

Data derived from Tan SY, Pepys MB. Amyloidosis. Histopathology, 25(5), 403-414 (November 1994), WHO/IUIS Nomenclature Subcommittee, Nomenclature of Amyloid and Amyloidosis. Bulletin of the World Health Organisation 1993; 71: 10508; and Merlini et al., Clin Chem Lab Med 2001; 39(11): 1065-75.

The data provided in Table 1 are exemplary and are not intended to limit the scope of the invention. For example, more than 40 separate point mutations in the transthyretin gene have been described, all of which give rise to clinically similar forms of familial amyloid polyneuropathy.

In general, any hereditary amyloid disorder can also occur sporadically, and both hereditary and sporadic forms of a disease present with the same characteristics with regard to amyloid. For example, the most prevalent form of secondary AA amyloidosis occurs sporadically, e.g. as a result of ongoing inflammation, and is not associated with Familial Mediterranean Fever. Thus general discussion relating to hereditary amyloid disorders below can also be applied to sporadic amyloidoses.

Transthyretin (TTR) is a 14 kiloDalton protein that is also sometimes referred to as prealbumin. It is produced by the liver and choroid plexus, and it functions in transporting thyroid hormones and vitamin A. At least 50 variant forms of the protein, each characterized by a single amino acid change, are responsible for various forms of familial amyloid polyneuropathy. For example, substitution of proline for leucine at position 55 results in a particularly progressive form of neuropathy; substitution of methionine for leucine at position 111 resulted in a severe cardiopathy in Danish patients.

Amyloid deposits isolated from heart tissue of patients with systemic amyloidosis have revealed that the deposits are composed of a heterogeneous mixture of TTR and fragments thereof, collectively referred to as ATTR, the full length sequences of which have been characterized. ATTR fibril components can be extracted from such plaques and their structure and sequence determined according to the methods known in the art (e.g., Gustavsson, A., et al., Laboratory Invest. 73: 703-708, 1995; Kametani, F., et al., Biochem. Biophys. Res. Commun. 125: 622-628, 1984; Pras, M., et al., PNAS 80: 539-42, 1983).

Persons having point mutations in the molecule apolipoprotein Al (e.g., Gly→Arg26; Trp→Arg50; Leu→Arg60) exhibit a form of amyloidosis (“Östertag type”) characterized by deposits of the protein apolipoprotein AI or fragments thereof (AApoAI). These patients have low levels of high density lipoprotein (HDL) and present with a peripheral neuropathy or renal failure.

A mutation in the alpha chain of the enzyme lysozyme (e.g., Ile→Thr56 or Asp→His57) is the basis of another form of Östertag-type non-neuropathic hereditary amyloid reported in English families. Here, fibrils of the mutant lysozyme protein (Alys) are deposited, and patients generally exhibit impaired renal function. This protein, unlike most of the fibril-forming proteins described herein, is usually present in whole (unfragmented) form (Benson, M. D., et al. CIBA Fdn. Symp. 199: 104-131, 1996).

Immunoglobulin light chains tend to form aggregates in various morphologies, including fibrillar (e.g., AL amyloidosis and AH amyloidosis), granular (e.g., light chain deposition disease (LCDD), heavy chain deposition disease (HCDD), and light-heavy chain deposition disease (LHCDD)), crystalline (e.g., Acquired Farconi's Syndome), and microtubular (e.g., Cryoglobulinemia). AL and AH amyloidosis is indicated by the formation of insoluble fibrils of immunoglobulin light chains and heavy chain, respectively, and/or their fragments. In AL fibrils, lambda (λ) chains such as λ VI chains (λ6 chains), are found in greater concentrations than kappa (κ) chains. λIII chains are also slightly elevated. Merlini et al., CLIN CHEM LAB MED 39(11):1065-75 (2001). Heavy chain amyloidosis (AH) is generally characterized by aggregates of gamma chain amyloid proteins of the IgG1 subclass. Eulitz et al., PROC NATL ACAD Sci USA 87:6542-46 (1990).

Comparison of amyloidogenic to non-amyloidogenic light chains has revealed that the former can include replacements or substitutions that appear to destabilize the folding of the protein and promote aggregation. AL and LCDD have been distinguished from other amyloid diseases due to their relatively small population monoclonal light chains, which are manufactured by neoplastic expansion of an antibody-producing B cell. AL aggregates typically are well-ordered fibrils of lambda chains. LCDD aggregates are relatively amorphous aggregations of both kappa and lambda chains, with a majority being kappa, in some cases κIV. Bellotti et al., JOURNAL OF STRUCTURAL BIOLOGY 13:280-89 (2000). Comparison of amyloidogenic and non-amyloidogenic heavy chains in patients having AH amyloidosis has revealed missing and/or altered components. Eulitz et al., PROC NATL ACAD SCI USA 87:6542-46 (1990) (pathogenic heavy chain characterized by significantly lower molecular mass than non-amyloidogenic heavy chains); and Solomon et al. AM J HEMAT 45(2) 171-6 (1994) (amyloidogenic heavy chain characterized as consisting solely of the VH-D portion of the non-amyloidogenic heavy chain).

Accordingly, potential methods of detecting and monitoring treatment of subjects having or at risk of having AL, LCDD, AH, and the like, include but are not limited to immunoassaying plasma or urine for the presence or depressed deposition of amyloidogenic light or heavy chains, e.g., amyloid λ, amyloid κ, amyloid κIV, amyloid γ, or amyloid γ1.

Brain Amyloidosis

The most frequent type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary. Nevertheless, fibril peptides forming plaques are very similar in both types. Brain amyloidosis includes those diseases, conditions, pathologies, and other abnormalities of the structure or function of the brain, including components thereof, in which the causative agent is amyloid. The area of the brain affected in an amyloid-related disease may be the stroma including the vasculature or the parenchyma including functional or anatomical regions, or neurons themselves. A subject need not have received a definitive diagnosis of a specifically recognized amyloid-related disease. The term “amyloid related disease” includes brain amyloidosis.

Amyloid-β peptide (“Aβ”) is a 39-43 amino acid peptide derived by proteolysis from a large protein known as Beta Amyloid Precursor Protein (“βAPP”). Mutations in βAPP result in familial forms of Alzheimer's disease, Down's syndrome, cerebral amyloid angiopathy, and senile dementia, characterized by cerebral deposition of plaques composed of Aβ fibrils and other components, which are described in further detail below. Known mutations in APP associated with Alzheimer's disease occur proximate to the cleavage sites of β or γ-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase in the amount of the 42/43 amino acid form of Aβ generated from APP. The familial form of Alzheimer's disease represents only 10% of the subject population. Most occurrences of Alzheimer's disease are sporadic cases where APP and Aβ do not possess any mutation. The structure and sequence of Aβ peptides of various lengths are well known in the art. Such peptides can be made according to methods known in the art, or extracted from the brain according to known methods (e.g., Glenner and Wong, Biochem. Biophys. Res. Comm. 129, 885-90 (1984); Glenner and Wong, Biochem. Biophys. Res. Comm. 122, 1131-35 (1984)). In addition, various forms of the peptides are commercially available. APP is expressed and constitutively catabolized in most cells. The dominant catabolic pathway appears to be cleavage of APP within the Aβ sequence by an enzyme provisionally termed α-secretase, leading to release of a soluble ectodomain fragment known as APPsα. This cleavage precludes the formation of Aβ peptide. In contrast to this non-amyloidogenic pathway, APP can also be cleaved by enzymes known as β- and γ-secretase at the N- and C-termini of the Aβ, respectively, followed by release of Aβ into the extracellular space. To date, BACE has been identified as β-secretase (Vasser, et al., Science 286:735-741, 1999) and presenilins have been implicated in γ-secretase activity (De Strooper, et al., Nature 391, 387-90 (1998)). The 39-43 amino acid Aβ peptide is produced by sequential proteolytic cleavage of the amyloid precursor protein (APP) by the β and γ secretases enzyme. Although Aβ40 is the predominant form produced, 5-7% of total Aβ exists as Aβ42 (Cappai et al., Int. J. Biochem. Cell Biol. 31. 885-89 (1999)).

The length of the Aβ peptide appears to dramatically alter its biochemical/biophysical properties. Specifically, the additional two amino acids at the C-terminus of Aβ42 are very hydrophobic, presumably increasing the propensity of Aβ42 to aggregate. For example, Jarrett, et al. demonstrated that Aβ42 aggregates very rapidly in vitro compared to Aβ40, suggesting that the longer forms of Aβ may be the important pathological proteins that are involved in the initial seeding of the neuritic plaques in Alzheimer's disease (Jarrett, et al., Biochemistry 32, 4693-97 (1993); Jarrett, et al., Ann. N.Y. Acad. Sci. 695, 144-48 (1993)). This hypothesis has been further substantiated by the recent analysis of the contributions of specific forms of Aβ in cases of genetic familial forms of Alzheimer's disease (“FAD”). For example, the “London” mutant form of APP (APPV7171) linked to FAD selectively increases the production of Aβ 42/43 forms versus Aβ 40 (Suzuki, et al., Science 264, 1336-40 (1994)) while the “Swedish” mutant form of APP (APPK670N/M671L) increases levels of both Aβ40 and Aβ42/43 (Citron, et al., Nature 360, 672-674 (1992); Cai, et al., Science 259, 514-16, (1993)). Also, it has been observed that FAD-linked mutations in the Presenilin-1 (“PS1”) or Presenilin-2 (“PS2”) genes will lead to a selective increase in Aβ42/43 production but not Aβ40 (Borchelt, et al., Neuron 17, 1005-13 (1996)). This finding was corroborated in transgenic mouse models expressing PS mutants that demonstrate a selective increase in brain Aβ42 (Borchelt, op cit.; Duff, et al., Neurodegeneration 5(4), 293-98 (1996)). Thus the leading hypothesis regarding the etiology of Alzheimer's disease is that an increase in Aβ42 brain concentration due to an increased production and release of Aβ42 or a decrease in clearance (degradation or brain clearance) is a causative event in the disease pathology.

Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia. CAA may also be sporadic.

As used herein, the terms “β amyloid,” “amyloid-β,” and the like refer to amyloid β proteins or peptides, amyloid β precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ1-39, Aβ1-40, Aβ1-41, Aβ1-42, and Aβ1-43. For convenience of nomenclature, “Aβ1-42” may be referred to herein as “Aβ(1-42)” or simply as “Aβ42” or “Aβ₄₂” (and likewise for any other amyloid peptides discussed herein). As used herein, the terms “β amyloid,” “amyloid-β,” and “Aβ” are synonymous.

Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque). See, e.g., M P Lambert, et al., Proc. Nat'l Acad. Sci. USA 95, 6448-53 (1998). “Amyloidosis” or “amyloid disease” or “amyloid-related disease” refers to a pathological condition characterized by the presence of amyloid fibers. “Amyloid” is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra.

Gelsolin is a calcium binding protein that binds to fragments and actin filaments. Mutations at position 187 (e.g., Asp→Asn; Asp→Tyr) of the protein result in a form of hereditary systemic amyloidosis, usually found in patients from Finland, as well as persons of Dutch or Japanese origin. In afflicted individuals, fibrils formed from gelsolin fragments (Agel), usually consist of amino acids 173-243 (68 kDa carboxyterminal fragment) and are deposited in blood vessels and basement membranes, resulting in corneal dystrophy and cranial neuropathy which progresses to peripheral neuropathy, dystrophic skin changes and deposition in other organs. (Kangas, H., et al. Human Mol. Genet. 5(9): 1237-1243, 1996).

Other mutated proteins, such as mutant alpha chain of fibrinogen (AfibA) and mutant cystatin C (Acys) also form fibrils and produce characteristic hereditary disorders. AfibA fibrils form deposits characteristic of a nonneuropathic hereditary amyloid with renal disease; Acys deposits are characteristic of a hereditary cerebral amyloid angiopathy reported in Iceland (Isselbacher, Harrison's Principles of Internal Medicine, McGraw-Hill, San Francisco, 1995; Benson, et al.). In at least some cases, patients with cerebral amyloid angiopathy (CAA) have been shown to have amyloid fibrils containing a non-mutant form of cystatin C in conjunction with amyloid beta protein (Nagai, A., et al. Molec. Chem. Neuropathol. 33: 63-78, 1998).

Certain forms of prion disease are now considered to be heritable, accounting for up to 15% of cases, which were previously thought to be predominantly infectious in nature. (Baldwin, et al., in Research Advances in Alzheimer's Disease and Related Disorders, John Wiley and Sons, New York, 1995). In hereditary and sporadic prion disorders, patients develop plaques composed of abnormal isoforms of the normal prion protein (PrP^{SC}).

A predominant mutant isoform, PrP^{SC}, also referred to as AScr, differs from the normal cellular protein in its resistance to protease degradation, insolubility after detergent extraction, deposition in secondary lysosomes, post-translational synthesis, and high β-pleated sheet content. Genetic linkage has been established for at least five mutations resulting in Creutzfeldt-Jacob disease (CJD), Gerstmann-Sträussler-Scheinker syndrome (GSS), and fatal familial insomnia (FFI). (Baldwin, supra) Methods for extracting fibril peptides from scrapie fibrils, determining sequences and making such peptides are known in the art (e.g., Beekes, M., et al. J. Gen. Virol. 76: 2567-76, 1995).

For example, one form of GSS has been linked to a PrP mutation at codon 102, while telencephalic GSS segregates with a mutation at codon 117. Mutations at codons 198 and 217 result in a form of GSS in which neuritic plaques characteristic of Alzheimer's disease contain PrP instead of Aβ peptide. Certain forms of familial CJD have been associated with mutations at codons 200 and 210; mutations at codons 129 and 178 have been found in both familial CJD and FFI. (Baldwin, supra).

Cerebral Amyloidosis

Local deposition of amyloid is common in the brain, particularly in elderly individuals. The most frequent type of amyloid in the brain is composed primarily of Aβ peptide fibrils, resulting in dementia or sporadic (non-hereditary) Alzheimer's disease. The most common occurrences of cerebral amyloidosis are sporadic and not familial. For example, the incidence of sporadic Alzheimer's disease and sporadic CAA greatly exceeds the incidence of familial AD and CAA. Moreover, sporadic and familial forms of the disease cannot be distinguished from each other (they differ only in the presence or absence of an inherited genetic mutation); for example, the clinical symptoms and the amyloid plaques formed in both sporadic and familial AD are very similar, if not identical.

Cerebral amyloid angiopathy (CAA) refers to the specific deposition of amyloid fibrils in the walls of leptomingeal and cortical arteries, arterioles and veins. It is commonly associated with Alzheimer's disease, Down's syndrome and normal aging, as well as with a variety of familial conditions related to stroke or dementia (see Frangione et al., Amyloid: J. Protein Folding Disord. 8, Suppl. 1, 36-42 (2001)). CAA can occur sporadically or be hereditary.

Amyloid deposition, either systemic or focal, increases with age. For example, fibrils of wild type transthyretin (TTR) are commonly found in the heart tissue of elderly individuals. These may be asymptomatic, clinically silent, or may result in heart failure. Asymptomatic fibrillar focal deposits may also occur in the brain (Aβ), corpora amylacea of the prostate (β₂ microglobulin), joints and seminal vesicles.

Dialysis-Related Amyloidosis (DRA)

Plaques composed of β₂ microglobulin (β₂M) fibrils commonly develop in patients receiving long term hemodialysis or peritoneal dialysis. β₂ microglobulin is a 11.8 kiloDalton polypeptide and is the light chain of Class I MHC antigens, which are present on all nucleated cells. Under normal circumstances, β₂M is usually distributed in the extracellular space unless there is an impaired renal function, in which case β₂M is transported into tissues where it polymerizes to form amyloid fibrils. Failure of clearance such as in the case of impaired renal function, leads to deposition in the carpal tunnel and other sites (primarily in collagen-rich tissues of the joints). Unlike other fibril proteins, β₂M molecules are not produced by cleavage of a longer precursor protein and are generally present in unfragmented form in the fibrils. (Benson, supra). Retention and accumulation of this amyloid precursor has been shown to be the main pathogenic process underlying DRA. DRA is characterized by peripheral joint osteoarthropathy (e.g., joint stiffness, pain, swelling, etc.). Isoforms of β₂M, glycated β₂M, or polymers of β₂M in tissue are the most amyloidogenic form (as opposed to native β₂M). Unlike other types of amyloidosis, β₂M is confined largely to osteoarticular sites. Visceral depositions are rare. Occasionally, these deposits may involve blood vessels and other important anatomic sites.

Despite improved dialysis methods for removal of β₂M, the majority of patients have plasmatic β₂M concentrations that remain dramatically higher than normal. These elevated β₂M concentrations generally lead to Dialysis-Related Amyloidosis (DRA) and cormorbidities that contribute to mortality.

Islet Amyloid Polypeptide and Diabetes

Islet hyalinosis (amyloid deposition) was first described over a century ago as the presence of fibrous protein aggregates in the pancreas of patients with severe hyperglycemia (Opie, E L., J Exp. Med. 5: 397-428, 1901). Today, islet amyloid, composed predominantly of islet amyloid polypeptide (IAPP), or amylin, is a characteristic histopathological marker in over 90% of all cases of Type II diabetes (also known as Non-Insulin Dependent Diabetes, or NIDDM). These fibrillar accumulations result from the aggregation of the islet amyloid polypeptide (IAPP) or amylin, which is a 37 amino acid peptide, derived from a larger precursor peptide, called pro-IAPP.

IAPP is co-secreted with insulin in response to β-cell secretagogues. This pathological feature is not associated with insulin-dependent (Type I) diabetes and is a unifying characteristic for the heterogeneous clinical phenotypes diagnosed as NIDDM (Type II diabetes).

Longitudinal studies in cats and immunocytochemical investigations in monkeys have shown that a progressive increase in islet amyloid is associated with a dramatic decrease in the population of insulin-secreting β-cells and increased severity of the disease. More recently, transgenic studies have strengthened the relationship between IAPP plaque formation and β-cell apoptosis and dysfunction, indicating that amyloid deposition is a principal factor in increasing severity of Type II diabetes.

IAPP has also been shown to induce β-islet cell toxicity in vitro, indicating that appearance of IAPP fibrils in the pancreas of Type II or Type I diabetic patients (post-islet transplantation) could contribute to the loss of the β-cell islets (Langerhans) and organ dysfunction. In patients with Type II diabetes, the accumulation of pancreatic IAPP leads to formation of oligomeric IAPP, leading to a buildup of IAPP-amyloid as insoluble fibrous deposits which eventually destroys the insulin-producing β cells of the islet, resulting in β cell depletion and failure (Westermark, P., Grimelius, L., Acta Path. Microbiol. Scand., sect. A. 81: 291-300, 1973; de Koning, E J P., et al., Diabetologia 36: 378-384, 1993; and Lorenzo, A., et al., Nature 368: 756-760, 1994). Accumulation of IAPP as fibrous deposits can also have an impact on the ratio of pro-IAPP to IAPP normally found in plasma by increasing this ratio due to the trapping of IAPP in deposits. Reduction of β cell mass can be manifested by hyperglycemia and insulinemia. This β-cell mass loss can lead to a need for insulin therapy.

Diseases caused by the death or malfunctioning of a particular type or types of cells can be treated by transplanting into the patient healthy cells of the relevant type of cell. This approach has been used for Type I diabetes patients. Often pancreatic islet cells from a donor are cultured in vitro prior to transplantation, to allow them to recover after the isolation procedure or to reduce their immunogenicity. However, in many instances islet cell transplantation is unsuccessful, due to death of the transplanted cells. One reason for this poor success rate is IAPP, which organizes into toxic oligomers. Toxic effects may result from intracellular and extracellular accumulation of fibril oligomers. The IAPP oligomers can form fibrils and become toxic to the cells in vitro. In addition, IAPP fibrils are likely to continue to grow after the cells are transplanted and cause death or dysfunction of the cells. This may occur even when the cells are from a healthy donor and the patient receiving the transplant does not have a disease that is characterized by the presence of fibrils. For example, compounds of the present invention may also be used in preparing tissues or cells for transplantation according to the methods described in International Patent Application (PCT) number WO 01/003680.

The compounds of the invention may also stabilize the ratio of the concentrations of Pro-IAPP/IAPP, pro-Insulin/Insulin and C-peptide levels. In addition, as biological markers of efficacy, the results of the different tests, such as the arginine-insulin secretion test, the glucose tolerance test, insulin tolerance and sensitivity tests, could all be used as markers of reduced β-cell mass and/or accumulation of amyloid deposits. Such class of drugs could be used together with other drugs targeting insulin resistance, hepatic glucose production, and insulin secretion. Such compounds might prevent insulin therapy by preserving β-cell function and be applicable to preserving islet transplants.

Hormone-Derived Amyloidoses

Endocrine organs may harbor amyloid deposits, particularly in aged individuals. Hormone-secreting tumors may also contain hormone-derived amyloid plaques, the fibrils of which are made up of polypeptide hormones such as calcitonin (medullary carcinoma of the thyroid), and atrial natriuretic peptide (isolated atrial amyloidosis). Sequences and structures of these proteins are well known in the art.

Miscellaneous Amyloidoses

There are a variety of other forms of amyloid disease that are normally manifest as localized deposits of amyloid. In general, these diseases are probably the result of the localized production or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid. Other amyloid related diseases include those described in Table 1, such as familial amyloid polyneuropathy (FAP), senile systemic amyloidosis, Tenosynovium, familial amyloidosis, Ostertag-type, non-neuropathic amyloidosis, cranial neuropathy, hereditary cerebral hemorrhage, familial dementia, chronic dialysis, familial Creutzfeldt-Jakob disease; Gerstmann-Sträussler-Scheinker syndrome, hereditary spongiform encephalopathies, prion diseases, familial Mediterranean fever, Muckle-Well's syndrome, nephropathy, deafness, urticaria, limb pain, cardiomyopathy, cutaneous deposits, multiple myeloma, benign monoclonal gammopathy, maccoglobulinaemia, myeloma associated amyloidosis, medullary carcinomas of the thyroid, isolated atrial amyloid, and diabetes.

The compounds of the invention may be administered therapeutically or prophylactically to treat diseases associated with amyloid fibril formation, aggregation or deposition, regardless of the clinical setting. The compounds of the invention may act to ameliorate the course of an amyloid related disease using any of the following mechanisms, such as, for example but not limited to: slowing the rate of amyloid fibril formation or deposition; lessening the degree of amyloid deposition; inhibiting, reducing, or preventing amyloid fibril formation; inhibiting amyloid induced inflammation; enhancing the clearance of amyloid from, for example, the brain; or protecting cells from amyloid induced (oligomers or fibrillar) toxicity.

In an embodiment, the compounds of the invention may be administered therapeutically or prophylactically to treat diseases associated with amyloid-β fibril formation, aggregation or deposition. The compounds of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; enhancing the clearance of amyloid-β from the brain; or favoring greater catabolism of Aβ.

Compounds of the invention may be effective in controlling amyloid-β deposition either following their entry into the brain (following penetration of the blood brain barrier) or from the periphery. When acting from the periphery, a compound may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. In addition, compounds that penetrate the brain may control deposition by acting directly on brain Aβ, e.g., by maintaining it in a non-fibrillar form or favoring its clearance from the brain. The compounds may slow down APP processing; may increase degradation of Aβ fibrils by macrophages or by neuronal cells; or may decrease Aβ production by activated microglia. These compounds could also prevent Aβ in the brain from interacting with the cell surface and therefore prevent neurotoxicity, neurodegeneration, or inflammation.

In a preferred embodiment, the method is used to treat Alzheimer's disease (e.g., sporadic or familial AD). The method can also be used prophylactically or therapeutically to treat other clinical occurrences of amyloid-β deposition, such as in Down's syndrome individuals and in patients with cerebral amyloid angiopathy (“CAA”), hereditary cerebral hemorrhage, or early Alzheimer's disease. According to certain aspects of the invention, amyloid-β is a peptide having 39-43 amino-acids, or amyloid-β is an amyloidogenic peptide produced from βAPP.

In another embodiment, the method is used to treat mild cognitive impairment. Mild Cognitive Impairment (“MCI”) is a condition characterized by a state of mild but measurable impairment in thinking skills, which is not necessarily associated with the presence of dementia. MCI frequently, but not necessarily, precedes Alzheimer's disease. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to “Mild-to-Moderate Cognitive Impairment,” as is well understood in this art.

Additionally, abnormal accumulation of APP and of amyloid-β protein in muscle fibers has been implicated in the pathology of sporadic inclusion body myositis (IBM) (Askanas, V., et al. (1996) Proc. Natl. Acad. Sci. USA 93: 1314-1319; Askanas, V. et al. (1995) Current Opinion in Rheumatology 7: 486-496). Accordingly, the compounds of the invention can be used prophylactically or therapeutically in the treatment of disorders in which amyloid-beta protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson, et al., Proc. Natl. Acad. Sci. USA 99(18), 11830-5 (2002)). Therefore, the invention also relates to the treatment or prevention of age-related macular degeneration.

In another embodiment, the invention also relates to a method of treating or preventing an amyloid-related disease in a subject (preferably a human) comprising administering to the subject a therapeutic amount of a compound according to the following Formulae or otherwise described herein, such that amyloid fibril formation or deposition, neurodegeneration, or cellular toxicity is reduced or inhibited. In another embodiment, the invention relates to a method of treating or preventing an amyloid-related disease in a subject (preferably a human) comprising administering to the subject a therapeutic amount of a compound according to the following Formulae or otherwise described herein, such that cognitive function is improved or stabilized or further deterioration in cognitive function is prevented, slowed, or stopped in patients with brain amyloidosis, e.g., Alzheimer's disease, Down's syndrome or cerebral amyloid angiopathy. These compounds can also improve quality of daily living in these subjects.

The therapeutic compounds of the invention may treat amyloidosis related to type II diabetes by, for example, stabilizing glycemia, preventing or reducing the loss of β cell mass, reducing or preventing hyperglycemia due to loss of β cell mass, and modulating (e.g., increasing or stabilizing) insulin production. The compounds of the invention may also stabilize the ratio of the concentrations of pro-IAPP/IAPP.

The therapeutic compounds of the invention may treat AA (secondary) amyloidosis and/or AL (primary) amyloidosis, by stabilizing renal function, decreasing proteinuria, increasing creatinine clearance (e.g., by at least 50% or greater or by at least 100% or greater), by leading to remission of chronic diarrhea or weight gain (e.g., 10% or greater), or by reducing serum creatinine. Visceral amyloid content as determined, e.g., by SAP scintigraphy may also be reduced.

Compounds of the Invention

The present invention relates, at least in part, to the use of certain chemical compounds (and pharmaceutical formulations thereof) in the prevention or treatment of amyloid-related diseases, including, inter alia, Alzheimer's disease, cerebral amyloid angiopathy, inclusion body myositis, Down's syndrome, diabetes related amyloidosis, hemodialysis-related amyloidosis (β₂M), primary amyloidosis (e.g., λ or κ chain-related), familial amyloid polyneuropathy (FAP), senile systemic amyloidosis, familial amyloidosis, Ostertag-type non-neuropathic amyloidosis, cranial neuropathy, hereditary cerebral hemorrhage, familial dementia, chronic dialysis, familial Creutzfeldt-Jakob disease, Gerstmann-Sträussler-Scheinker syndrome, hereditary spongiform encephalopathies, prion diseases, familial Mediterranean fever, Muckle-Well's syndrome, nephropathy, deafness, urticaria, limb pain, cardiomyopathy, cutaneous deposits, multiple myeloma, benign monoclonal gammopathy, maccoglobulinaemia, myeloma associated amyloidosis, medullary carcinomas of the thyroid, and isolated atrial amyloid.

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis. Furthermore, alkenes can include either the E- or Z-geometry, where appropriate. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention.

A “small molecule” refers to a compound that is not itself the product of gene transcription or translation (e.g., protein, RNA, or DNA) and preferably has a low molecular weight, e.g., less than about 2500 amu.

As used herein, “alkyl” groups include saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups). The term “aliphatic group” includes organic moieties characterized by straight or branched-chains, typically having between 1 and 22 carbon atoms. In complex structures, the chains may be branched, bridged, or cross-linked. Aliphatic groups include alkyl groups, alkenyl groups, and alkynyl groups.

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and more preferably 18 or fewer. Likewise, preferred cycloalkyl groups have from 4-10 carbon atoms in their ring structure, and more preferably have 4-7 carbon atoms in the ring structure. The term “lower alkyl” refers to alkyl groups having from 1 to 6 carbons in the chain, and to cycloalkyl groups having from 3 to 6 carbons in the ring structure.

Unless the number of carbons is otherwise specified, “lower” as in “lower aliphatic,” “lower alkyl,” “lower alkenyl,” etc. as used herein means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), and more preferably 4 or fewer. Likewise, preferred cycloalkyl groups have from 3-8 carbon atoms in their ring structure, and more preferably have 5 or 6 carbons in the ring structure. The term “C₁-C₆” as in “C₁-C₆ alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, or aromatic (including heteroaromatic) groups.

An “arylalkyl” group is an alkyl group substituted with an aryl group (e.g., phenylmethyl (i.e., benzyl)). An “alkylaryl” moiety is an aryl group substituted with an alkyl group (e.g., p-methylphenyl (i.e., p-tolyl)). The term “n-alkyl” means a straight-chain (i.e., unbranched) unsubstituted alkyl group. An “alkylene” group is a divalent analog of the corresponding alkyl group. The terms “alkenyl” and “alkynyl” refer to unsaturated aliphatic groups analogous to alkyls, but which contain at least one double or triple carbon-carbon bond respectively. Suitable alkenyl and alkynyl groups include groups having 2 to about 12 carbon atoms, preferably from 2 to about 6 carbon atoms.

The term “aromatic group” or “aryl group” includes unsaturated and aromatic cyclic hydrocarbons as well as unsaturated and aromatic heterocycles containing one or more rings. Aryl groups may also be fused or bridged with alicyclic or heterocyclic rings that are not aromatic so as to form a polycycle (e.g., tetralin). An “arylene” group is a divalent analog of an aryl group. Aryl groups can also be fused or bridged with alicyclic or heterocyclic rings which are not aromatic so as to form a polycycle (e.g., tetralin).

The term “heterocyclic group” includes closed ring structures analogous to carbocyclic groups in which one or more of the carbon atoms in the ring is an element other than carbon, for example, nitrogen, sulfur, or oxygen. Heterocyclic groups may be saturated or unsaturated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups.

Unless otherwise stipulated, aryl and heterocyclic (including heteroaryl) groups may also be substituted at one or more constituent atoms. Examples of heteroaromatic and heteroalicyclic groups may have 1 to 3 separate or fused rings with 3 to about 8 members per ring and one or more N, O, or S heteroatoms. In general, the term “heteroatom” includes atoms of any element other than carbon or hydrogen, preferred examples of which include nitrogen, oxygen, sulfur, and phosphorus. Heterocyclic groups may be saturated or unsaturated or aromatic.

Examples of heterocycles include, but are not limited to, acridinyl; azocinyl; benzimidazolyl; benzofuranyl; benzothiofuranyl; benzothiophenyl; benzoxazolyl; benzthiazolyl; benztriazolyl; benztetrazolyl; benzisoxazolyl; benzisothiazolyl; benzimidazolinyl; carbazolyl; 4aH-carbazolyl; carbolinyl; chromanyl; chromenyl; cinnolinyl; decahydroquinolinyl; 2H,6H-1,5,2-dithiazinyl; dihydrofuro[2,3-b]tetrahydrofuran; furanyl; furazanyl; imidazolidinyl; imidazolinyl; imidazolyl; 1H-indazolyl; indolenyl; indolinyl; indolizinyl; indolyl; 3H-indolyl; isobenzofuranyl; isochromanyl; isoindazolyl; isoindolinyl; isoindolyl; isoquinolinyl; isothiazolyl; isoxazolyl; methylenedioxyphenyl; morpholinyl; naphthyridinyl; octahydroisoquinolinyl; oxadiazolyl; 1,2,3-oxadiazolyl; 1,2,4-oxadiazolyl; 1,2,5-oxadiazolyl; 1,3,4-oxadiazolyl; oxazolidinyl; oxazolyl; oxazolidinyl; pyrimidinyl; phenanthridinyl; phenanthrolinyl; phenazinyl; phenothiazinyl; phenoxathiinyl; phenoxazinyl; phthalazinyl; piperazinyl; piperidinyl; piperidonyl; 4-piperidonyl; piperonyl; pteridinyl; purinyl; pyranyl; pyrazinyl; pyrazolidinyl; pyrazolinyl; pyrazolyl; pyridazinyl; pyridooxazole; pyridoimidazole; pyridothiazole; pyridinyl; pyridyl; pyrimidinyl; pyrrolidinyl; pyrrolinyl; 2H-pyrrolyl; pyrrolyl; quinazolinyl; quinolinyl; 4H-quinolizinyl; quinoxalinyl; quinuclidinyl; tetrahydrofuranyl; tetrahydroisoquinolinyl; tetrahydroquinolinyl; tetrazolyl; 6H-1,2,5-thiadiazinyl; 1,2,3-thiadiazolyl; 1,2,4-thiadiazolyl; 1,2,5-thiadiazolyl; 1,3,4-thiadiazolyl; thianthrenyl; thiazolyl; thienyl; thienothiazolyl; thienooxazolyl; thienoimidazolyl; thiophenyl; triazinyl; 1,2,3-triazolyl; 1,2,4-triazolyl; 1,2,5-triazolyl; 1,3,4-triazolyl; and xanthenyl. Preferred heterocycles include, but are not limited to, pyridinyl; furanyl; thienyl; pyrrolyl; pyrazolyl; pyrrolidinyl; imidazolyl; indolyl; benzimidazolyl; 1H-indazolyl; oxazolidinyl; benzotriazolyl; benzisoxazolyl; oxindolyl; benzoxazolinyl; and isatinoyl groups. Also included are fused ring and spiro compounds containing, for example, the above heterocycles.

A common hydrocarbon aryl group is a phenyl group having one ring. Two-ring hydrocarbon aryl groups include naphthyl, indenyl, benzocyclooctenyl, benzocycloheptenyl, pentalenyl, and azulenyl groups, as well as the partially hydrogenated analogs thereof such as indanyl and tetrahydronaphthyl. Exemplary three-ring hydrocarbon aryl groups include acephthylenyl, fluorenyl, phenalenyl, phenanthrenyl, and anthracenyl groups.

Aryl groups also include heteromonocyclic aryl groups, i.e., single-ring heteroaryl groups, such as thienyl, furyl, pyranyl, pyrrolyl, imidazolyl, pyrazolyl, pyridinyl, pyrazinyl, pyrimidinyl, and pyridazinyl groups; and oxidized analogs thereof such as pyridonyl, oxazolonyl, pyrazolonyl, isoxazolonyl, and thiazolonyl groups. The corresponding hydrogenated (i.e., non-aromatic) heteromonocylic groups include pyrrolidinyl, pyrrolinyl, imidazolidinyl, imidazolinyl, pyrazolidinyl, pyrazolinyl, piperidyl and piperidino, piperazinyl, and morpholino and morpholinyl groups.

Aryl groups also include fused two-ring heteroaryls such as indolyl, isoindolyl, indolizinyl, indazolyl, quinolinyl, isoquinolinyl, phthalazinyl, quinoxalinyl, quinazolinyl, cinnolinyl, chromenyl, isochromenyl, benzothienyl, benzimidazolyl, benzothiazolyl, purinyl, quinolizinyl, isoquinolonyl, quinolonyl, naphthyridinyl, and pteridinyl groups, as well as the partially hydrogenated analogs such as chromanyl, isochromanyl, indolinyl, isoindolinyl, and tetrahydroindolyl groups. Aryl groups also include fused three-ring groups such as phenoxathiinyl, carbazolyl, phenanthridinyl, acridinyl, perimidinyl, phenanthrolinyl, phenazinyl, phenothiazinyl, phenoxazinyl, and dibenzofuranyl groups.

Some typical aryl groups include substituted or unsubstituted 5- and 6-membered single-ring groups. In another aspect, each Ar group may be selected from the group consisting of substituted or unsubstituted phenyl, pyrrolyl, furyl, thienyl, thiazolyl, isothiaozolyl, imidazolyl, triazolyl, tetrazolyl, pyrazolyl, oxazolyl, isooxazolyl, pyridinyl, pyrazinyl, pyridazinyl, and pyrimidinyl groups. Further examples include substituted or unsubstituted phenyl, 1-naphthyl, 2-naphthyl, biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl groups.

The term “amine” or “amino,” as used herein, refers to an unsubstituted or substituted moiety of the formula —NR^{a}R^{b}, in which R^{a} and R^{b} are each independently hydrogen, alkyl, aryl, or heterocyclyl, or R^{a} and R^{b}, taken together with the nitrogen atom to which they are attached, form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, preferably from 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino” and “diarylamino” include groups wherein the nitrogen is bound to at least one or two aryl groups, respectively. The term “alkylarylamino” refers to an amino group which is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties which contain a nitrogen atom which is bound to the carbon of a carbonyl or a thiocarbonyl group.

The term “alkylthio” refers to an alkyl group, having a sulfhydryl group attached thereto. Suitable alkylthio groups include groups having 1 to about 12 carbon atoms, preferably from 1 to about 6 carbon atoms.

The term “alkylcarboxyl” as used herein means an alkyl group having a carboxyl group attached thereto.

The term “alkoxy” as used herein means an alkyl group having an oxygen atom attached thereto. Representative alkoxy groups include groups having 1 to about 12 carbon atoms, preferably 1 to about 6 carbon atoms, e.g., methoxy, ethoxy, propoxy, tert-butoxy and the like. Examples of alkoxy groups include methoxy, ethoxy, isopropyloxy, propoxy, butoxy, and pentoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain an oxygen bonded to two carbon atoms. For example, an ether or ethereal group includes “alkoxyalkyl” which refers to an alkyl, alkenyl, or alkynyl group substituted with an alkoxy group.

A “sulfonate” group is a —SO₃H or —SO₃⁻X⁺ group bonded to a carbon atom, where X⁺ is a cationic counter ion group. Similarly, a “sulfonic acid” compound has a —SO₃H or —SO₃⁻X⁺ group bonded to a carbon atom, where X+ is a cationic group. A “sulfate” as used herein is a —OSO₃H or —OSO₃⁻X⁺ group bonded to a carbon atom, and a “sulfuric acid” compound has a —SO₃H or —OSO₃⁻X⁺ group bonded to a carbon atom, where X⁺ is a cationic group. According to the invention, a suitable cationic group may be a hydrogen atom. In certain cases, the cationic group may actually be another group on the therapeutic compound that is positively charged at physiological pH, for example an amino group.

A “counter ion” is required to maintain electroneutrality. Examples of anionic counter ions include halide, triflate, sulfate, nitrate, hydroxide, carbonate, bicarbonate, acetate, phosphate, oxalate, cyanide, alkylcarboxylate, N-hydroxysuccinimide, N-hydroxybenzotriazole, alkoxide, thioalkoxide, alkane sulfonyloxy, halogenated alkane sulfonyloxy, arylsulfonyloxy, bisulfate, oxalate, valerate, oleate, palmitate, stearate, laurate, borate, benzoate, lactate, citrate, maleate, fumarate, succinate, tartrate, naphthylate mesylate, glucoheptonate, or lactobionate. Compounds containing a cationic group covalently bonded to an anionic group may be referred to as an “internal salt.”

The term “nitro” means —NO₂; the term “halogen” or “halogeno” or “halo” designates —F, —Cl, —Br or —I; the term “thiol,” “thio,” or “mercapto” means SH; and the term “hydroxyl” or “hydroxy” means —OH.

The term “acyl” refers to a carbonyl group that is attached through its carbon atom to a hydrogen (i.e., a formyl), an aliphatic group (e.g., acetyl), an aromatic group (e.g., benzoyl), and the like. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless otherwise specified, the chemical moieties of the compounds of the invention, including those groups discussed above, may be “substituted or unsubstituted.” In some embodiments, the term “substituted” means that the moiety has substituents placed on the moiety other than hydrogen (i.e., in most cases, replacing a hydrogen), which allow the molecule to perform its intended function. Examples of substituents include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, and heteroaryl groups, as well as (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), and (CR′R″)₀₋₃OR′ groups, wherein R′ and R″ are each independently hydrogen, a C₁-C₅ alkyl, C₂-C₅ alkenyl, C₂-C₅ alkynyl, or aryl group; or the side chain of any naturally occurring amino acid.

In another embodiment, a substituent may be selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂, (CR′R″)₀₋₁₀OCH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₂(CR′R″)₀₋₁₀H, (CR′R″)₀₋₁₀S(O)₀₋₃R′ (e.g., —SO₃H), (CR′R″)₀₋₁₀O(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₀₋₁₀OH (e.g., —OH), (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′ (e.g., —CO₂H), or (CR′R″)₀₋₁₀OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more.

In some embodiments, a “substituent” may be selected from the group consisting of, for example, halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl (including heteroaryl) groups.

In one embodiment, the invention pertains to compounds of Formula I:

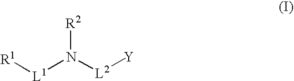

wherein:

R¹ is fluorine, hydrogen, a substituted or unsubstituted cycloalkyl, a substituted or unsubstituted aryl, a substituted or unsubstituted acyl, a substituted or unsubstituted arylcycloalkyl, a substituted or unsubstituted bicyclic or tricyclic ring, a bicyclic or tricyclic fused ring group, or a substituted or unsubstituted C₂-C₁₀ alkyl group;

R² is hydrogen, fluorine, a substituted or unsubstituted acyl, a substituted or unsubstituted alkyl, a substituted or unsubstituted mercaptoalkyl, a substituted or unsubstituted alkenyl, a substituted or unsubstituted alkynyl, a substituted or unsubstituted cycloalkyl, a substituted or unsubstituted aryl, a substituted or unsubstituted arylalkyl, a substituted or unsubstituted thiazolyl, a substituted or unsubstituted triazolyl, a substituted or unsubstituted imidazolyl, a substituted or unsubstituted benzothiazolyl, or a substituted or unsubstituted benzoimidazolyl;

Y is SO₃⁻X⁺, OSO₃⁻X⁺, SSO₃⁻X⁺, SO₂⁻X⁺, or CO₂⁻X⁺;

X⁺ is hydrogen or a cationic group; and

L¹ and L² are each independently a substituted or unsubstituted C₁-C₁₂ alkyl group or absent;

and pharmaceutically acceptable salts, esters or prodrugs thereof, provided that at least one of R¹, R², L¹, or L² comprise one or more fluorine atoms, provided that when L² comprises one fluorine atom and Y is SO₂⁻X⁺, at least one of R¹ and R² is not hydrogen; and provided that when Y is CO₂⁻X⁺, and L² is C₂ substituted with an aryl group, then at least one of R¹ and R² is not hydrogen.

In another embodiment, R¹ is fluorine or hydrogen. In another alternate embodiment, R¹ is a substituted or unsubstituted C₂-C₁₀ alkyl group. The substituted alkyl group may be substituted with any substituent that allows it to perform its intended function. In another embodiment, R¹ is a cyclic alkyl group. Examples of cyclic alkyl groups of the invention include, but are not limited to, cyclobutyl, cyclopentyl, and cyclohexyl.

In another embodiment, R¹ is fluorinated methyl (e.g., CH₂F, CHF₂, or CF₃), fluorinated ethyl (e.g., C₂F₅, C₂HF₄, C₂H₂F₃, C₂H₃F₂, or C₂H₄F), fluorinated propyl, fluorinated butyl, fluorinated pentyl, or fluorinated heptyl. L¹ may be absent when R¹ is fluorine, hydrogen or lower alkyl. In another embodiment, R¹ is fluorinated acyl. Examples of fluorinated acyl groups include C(═O)CH₂F, C(═O)CHF₂, C(═O)CF₃, C(═O)C₂F₅, C(═O)C₂HF₄, C(═O)C₂H₂F₃, C(═O)C₂H₃F₂, and C(═O)C₂H₄F. Other examples of R¹ groups include those exemplified in U.S. Ser. No. 10/871,514, filed on Jun. 18, 2004. In another embodiment, R¹ is a fluorinated benzaldehyde moiety.

In another embodiment, R¹ is an aryl group (e.g., phenyl, pyrrolyl, furyl, thienyl, etc.). In yet another embodiment, R¹ is a phenyl substituted with fluorine, trifluoromethyl, alkyl (e.g., methyl, ethyl, propyl, butyl) or a combination thereof. In another embodiment, R¹ is 4-fluorophenyl. In another embodiment R¹ is a substituted or unsubstituted bicyclic fused ring moiety (e.g., indolyl, isoquinolinyl, phthalazinyl, etc.). In a further embodiment, R¹ is 2,3-dihydro-1H-indene, which can optionally be substituted with fluorine.

In another further embodiment, R² is fluorine or hydrogen. In another alternate embodiment, R² is a substituted or unsubstituted C₂-C₁₀ alkyl group. The substituted alkyl group may be substituted with any substituent that allows it to perform its intended function. In a further embodiment, R² is fluorinated lower alkyl. In another embodiment, R² is fluorinated methyl (e.g., CH₂F, CHF₂, or CF₃), fluorinated ethyl (e.g., C₂F₅, C₂HF₄, C₂H₂F₃, C₂H₃F₂, or C₂H₄F), fluorinated propyl, fluorinated butyl, fluorinated pentyl, or fluorinated heptyl. In another embodiment, R² is fluorinated acyl. Examples of fluorinated acyl groups include C(═O)CH₂F, C(═O)CHF₂, C(═O)CF₃, C(═O)C₂F₅, C(═O)C₂HF₄, C(═O)C₂H₂F₃, C(═O)C₂H₃F₂, and C(═O)C₂H₄F. Other examples of R² groups include those exemplified in U.S. Ser. No. 10/871,514, filed on Jun. 18, 2004. In a further embodiment, R² is fluorinated lower alkyl.

In another embodiment, R² is an aryl group. An example of an aryl group includes but is not limited to a phenyl group. In another embodiment, L² may be a C₁-C₃ alkyl when R² is an aryl group.

In yet another embodiment, Y is SO₃⁻X⁺, SO₂⁻X⁺, or CO₂X⁻.

In another, L² is a C₂-C₈ substituted or unsubstituted alkyl moiety. In a further embodiment, L² is a substituted or unsubstituted C₂-C₅ alkyl moiety. Examples of L² include, but are not limited to, —(CH₂)₂—, —(CH₂)₃—, and —(CH₂)₄—. In another embodiment, L² is substituted with a fluorinated ester moiety. In other embodiment, L² is substituted with one, two, three, four or five fluorine atoms.

In another embodiment, L¹ is C₁₋₄ alkyl. In a further embodiment, L¹ is CH₂, C(CH₃)₂, or CH(CH₃). In another embodiment, R¹ and R² are each hydrogen, and L¹ is absent. In another embodiment, L² is ethyl or propyl and substituted by one or more fluorines (e.g., —(CH₂)₁₋₂—CF₂—).

In a further embodiment, Y is SO₃⁻X⁺ and L² is —(CH₂)₃—. In this embodiment, R² may be hydrogen and L¹ may be alkyl, e.g., unsubstituted or branched alkyl, e.g., —CH(CH₃)CH₂. Furthermore, R¹ may be substituted or unsubstituted aryl, e.g., substituted or unsubstituted phenyl. In a further embodiment, the phenyl is para-substituted, e.g., para-substituted with fluorine.

In a further embodiment, the compound is selected from the group consisting of: 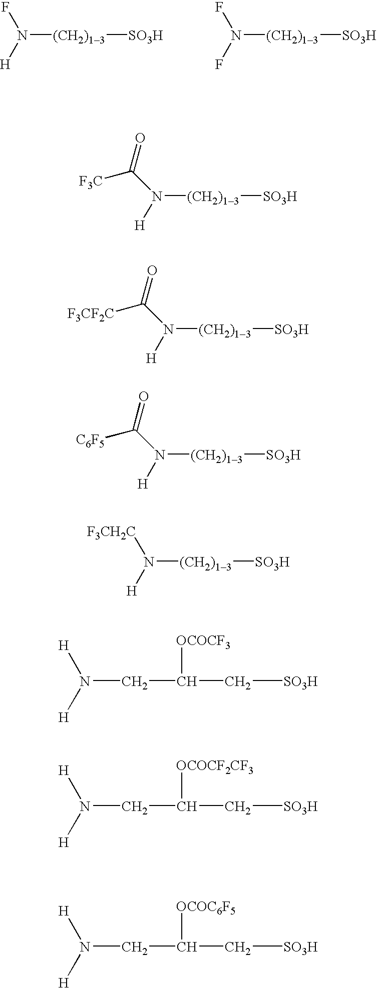
and pharmaceutically acceptable salts, esters, or prodrugs thereof.

In another embodiment, the compound is selected from the group consisting of: 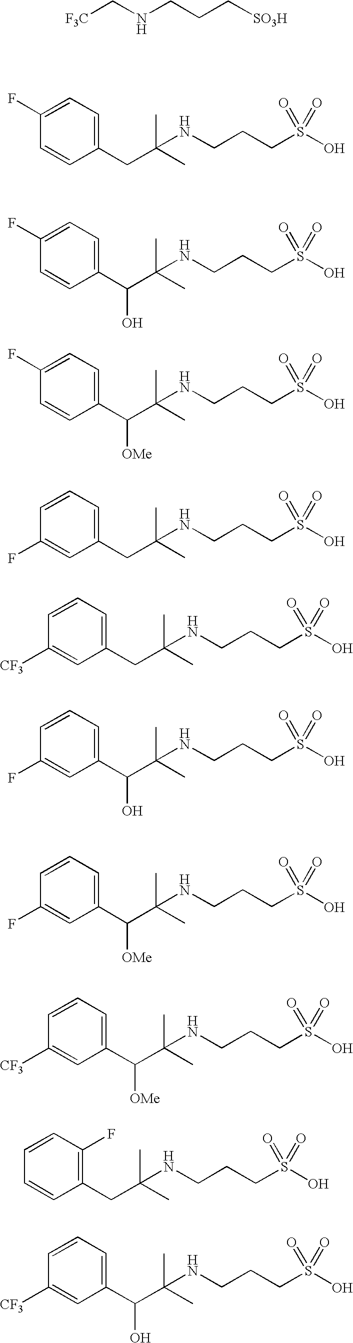 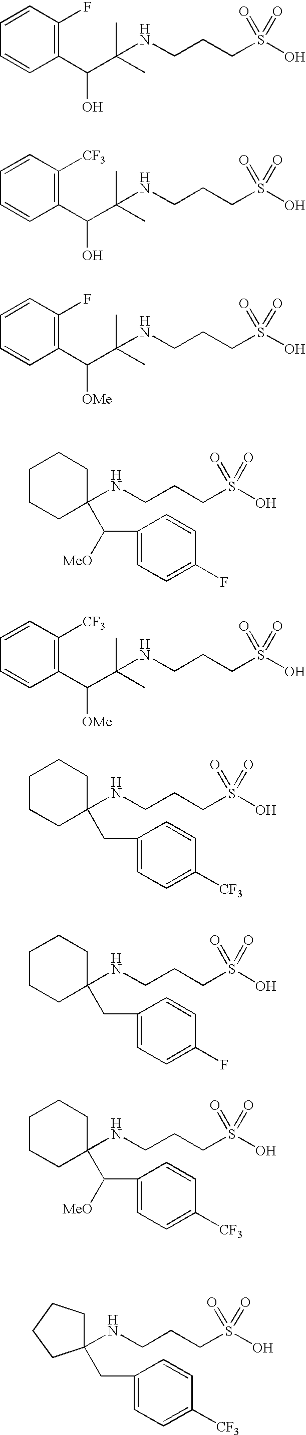 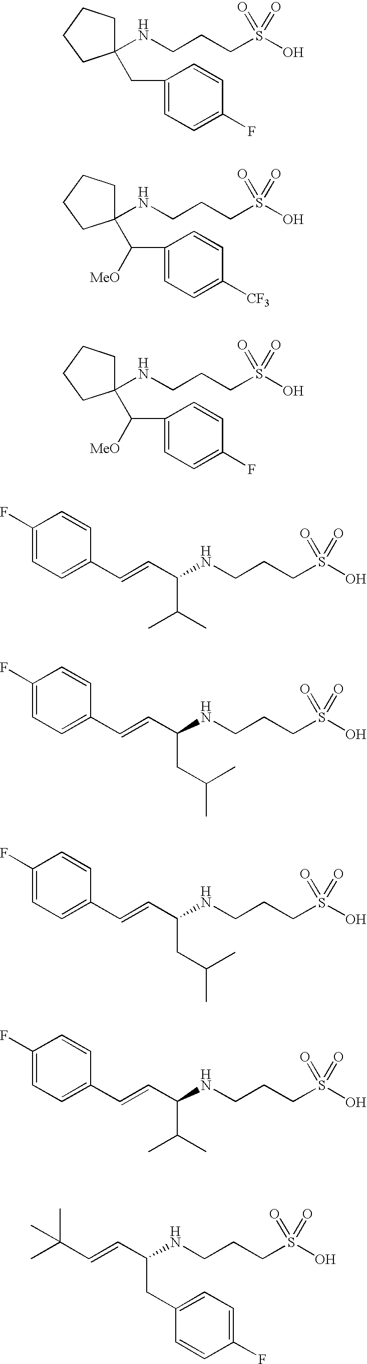 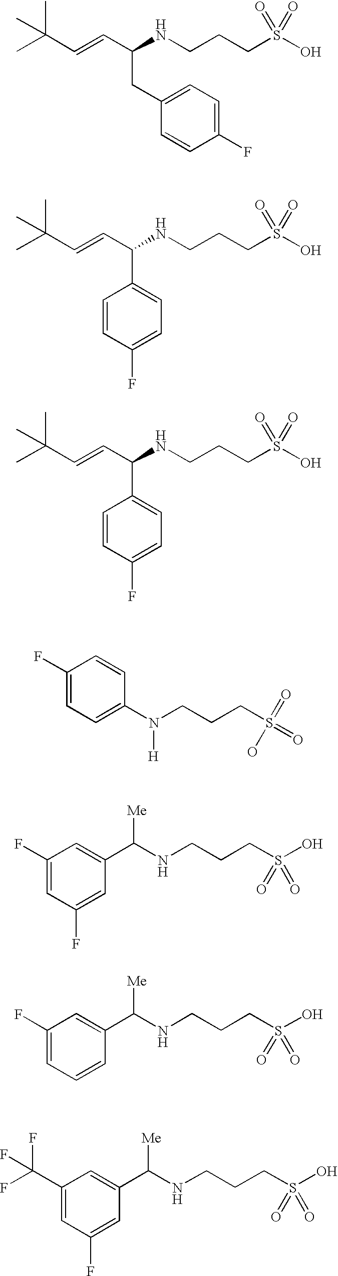 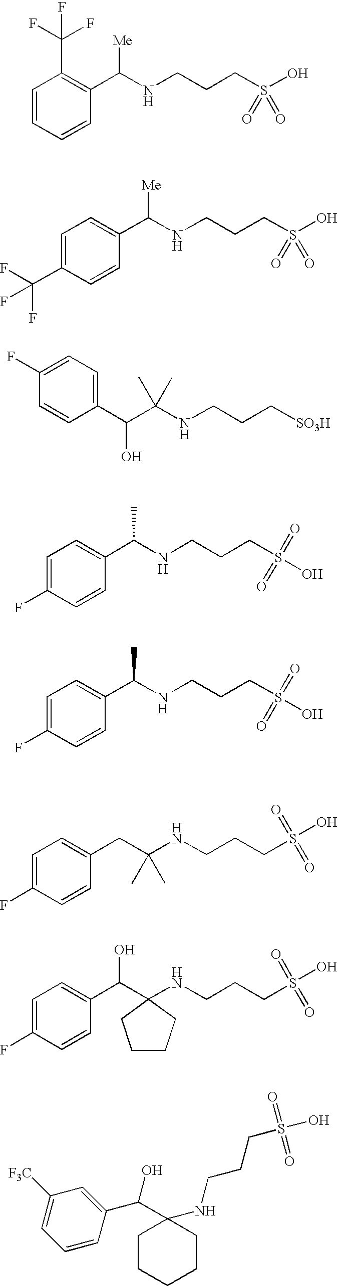 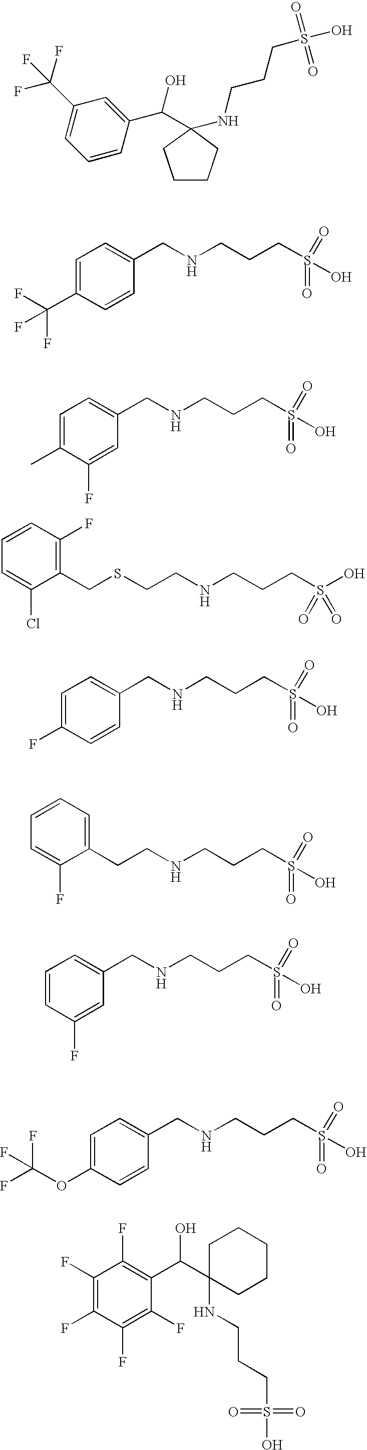 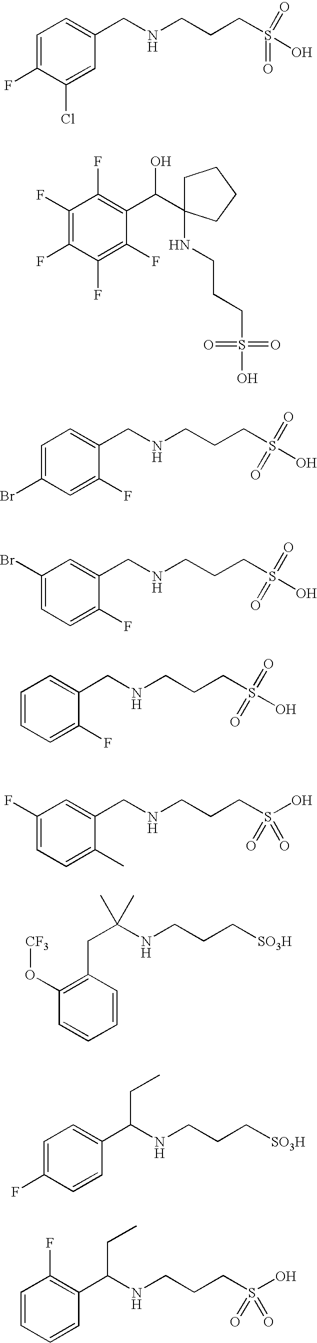 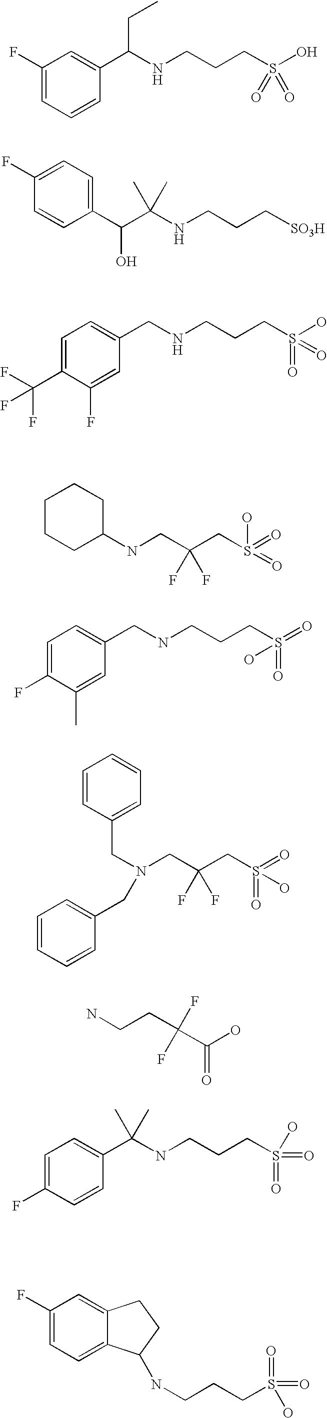 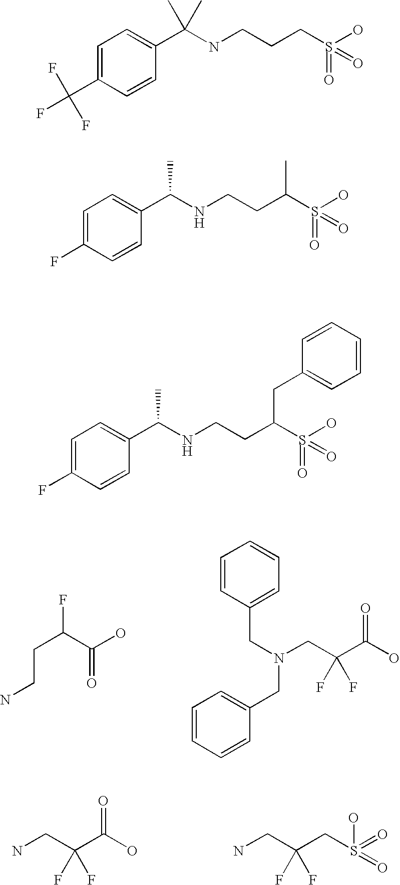
and pharmaceutically acceptable salts, esters, or prodrugs thereof.

In another embodiment, the compounds of the invention include: 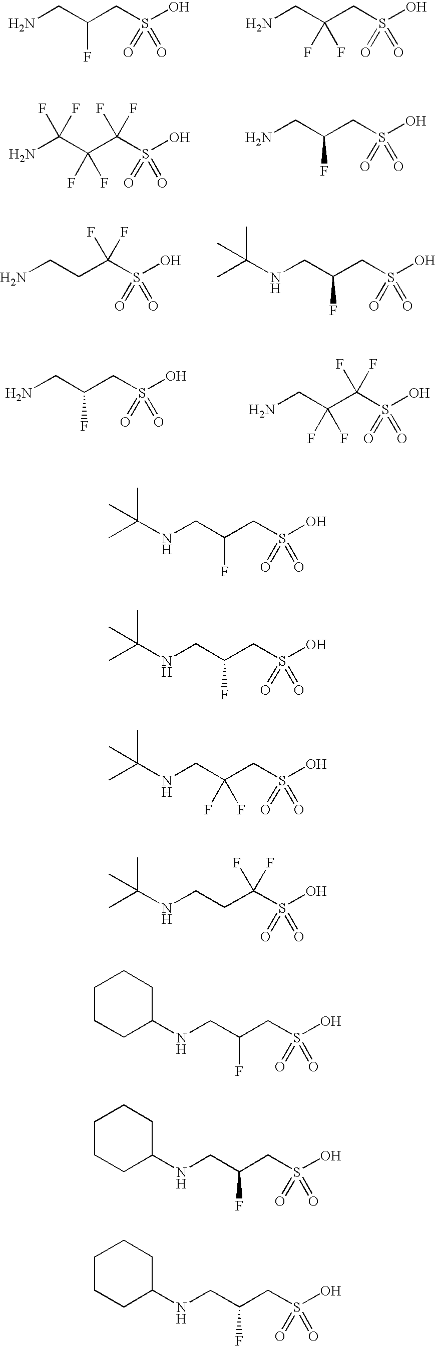 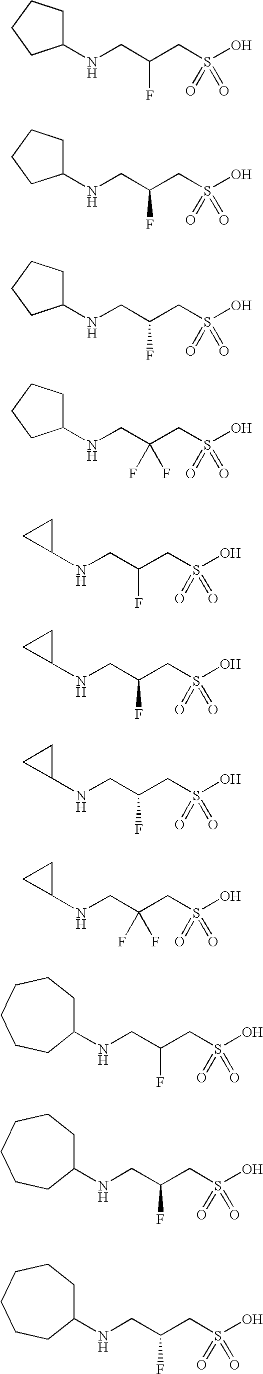 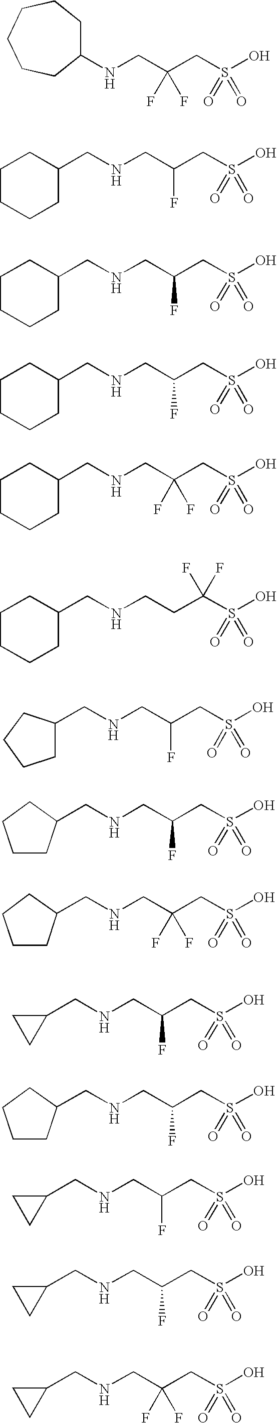 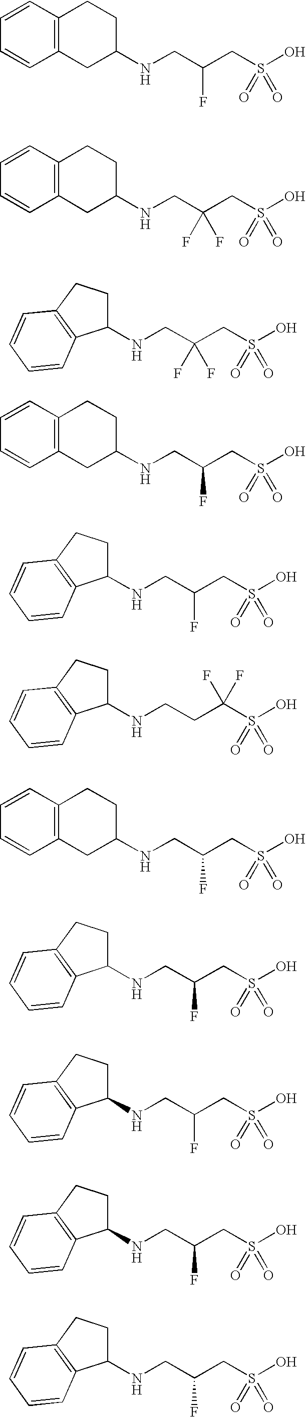 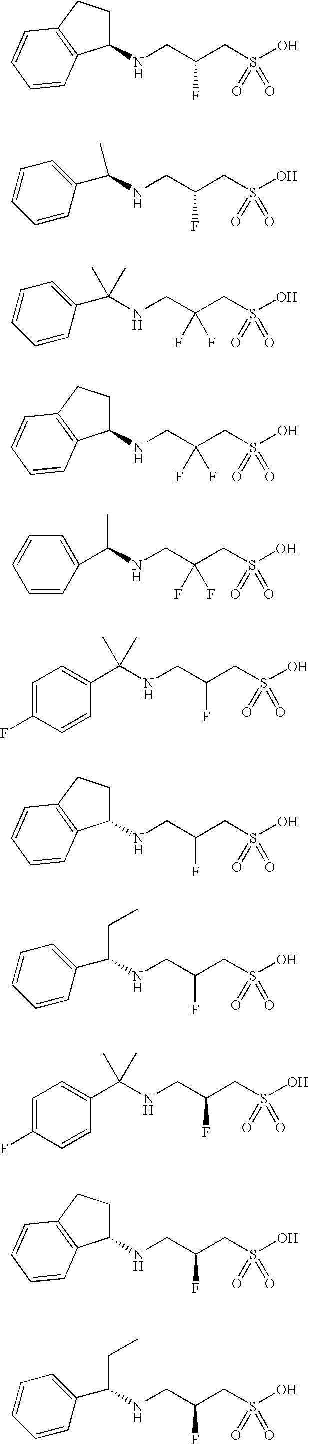 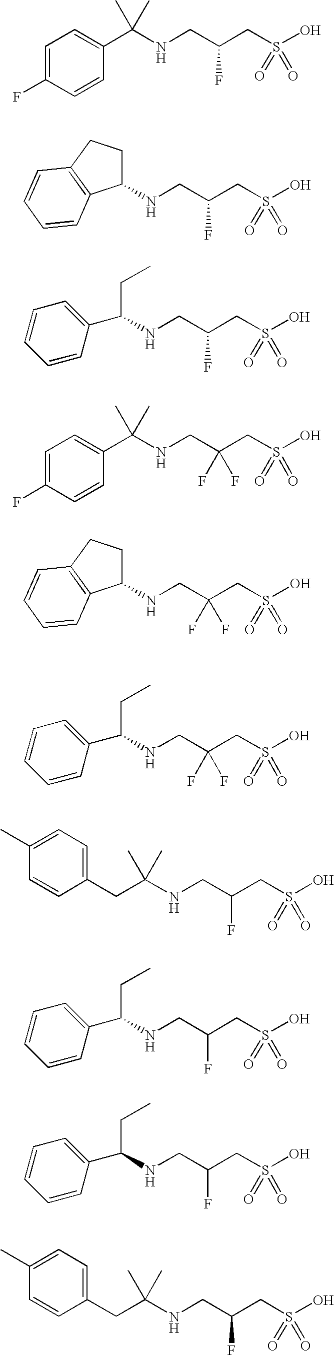 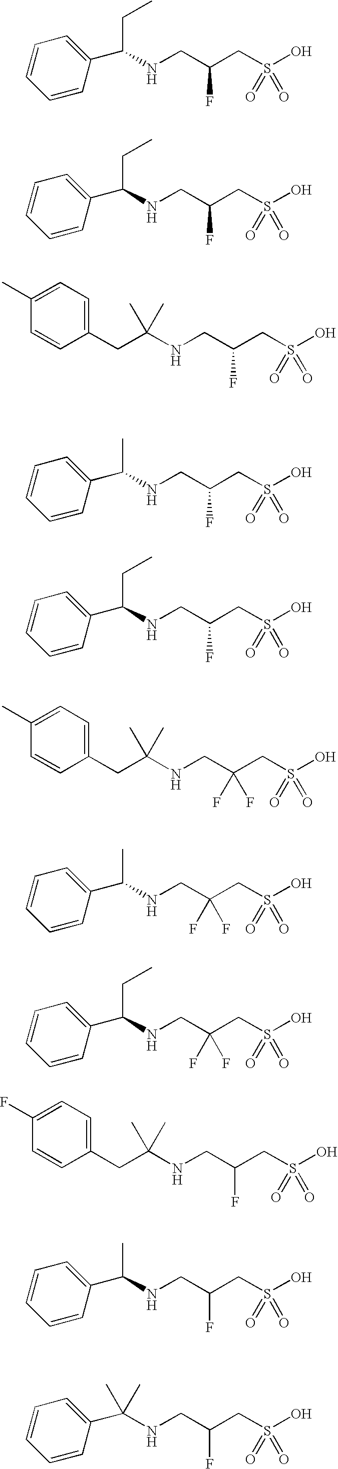 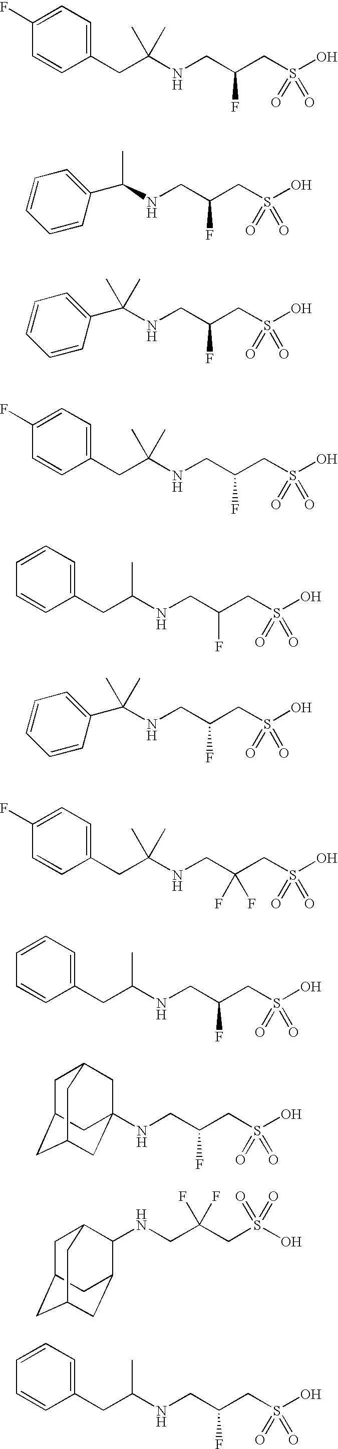 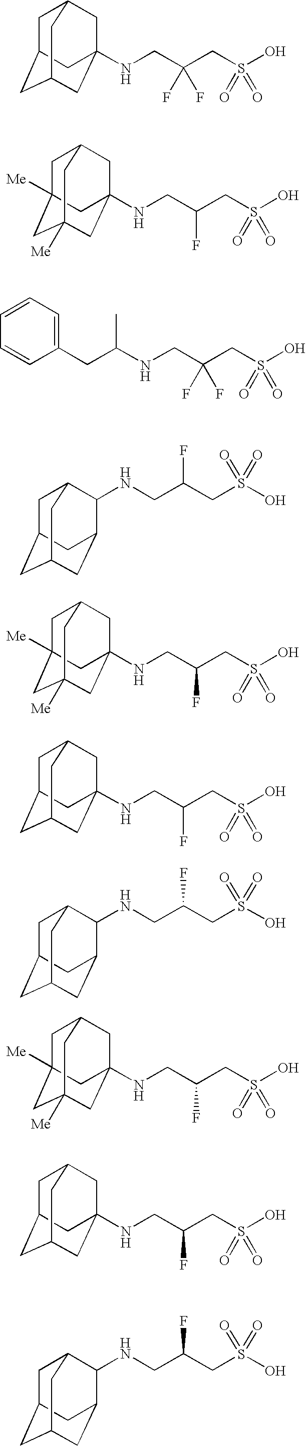 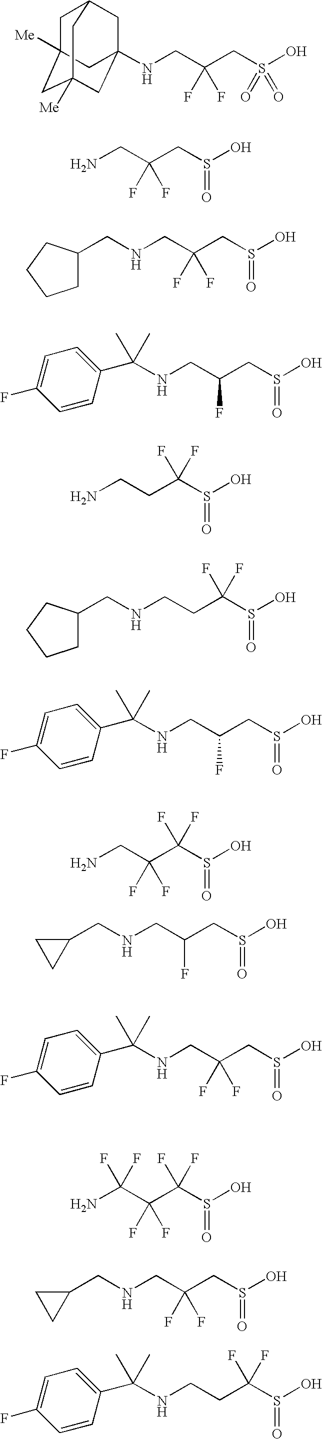 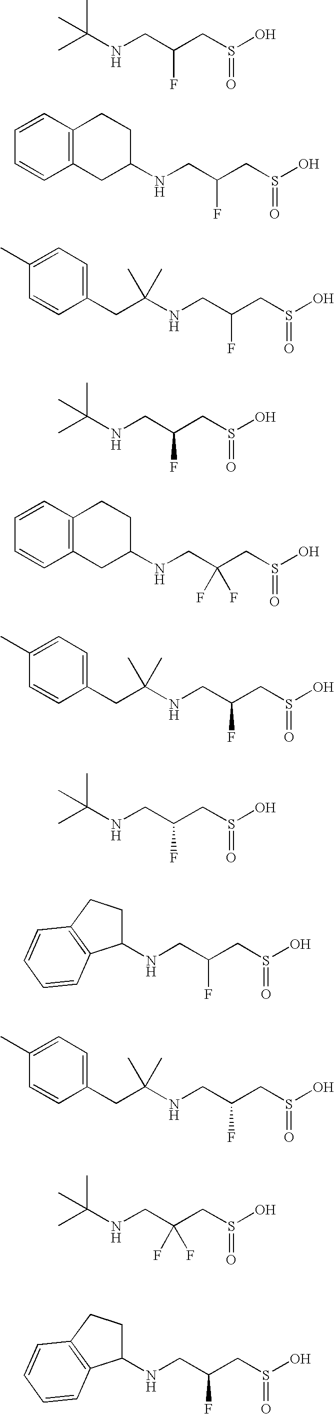 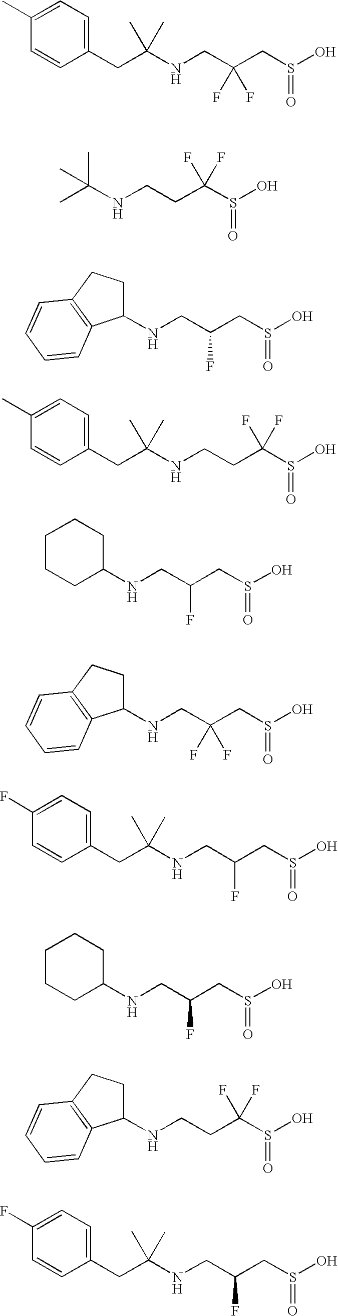 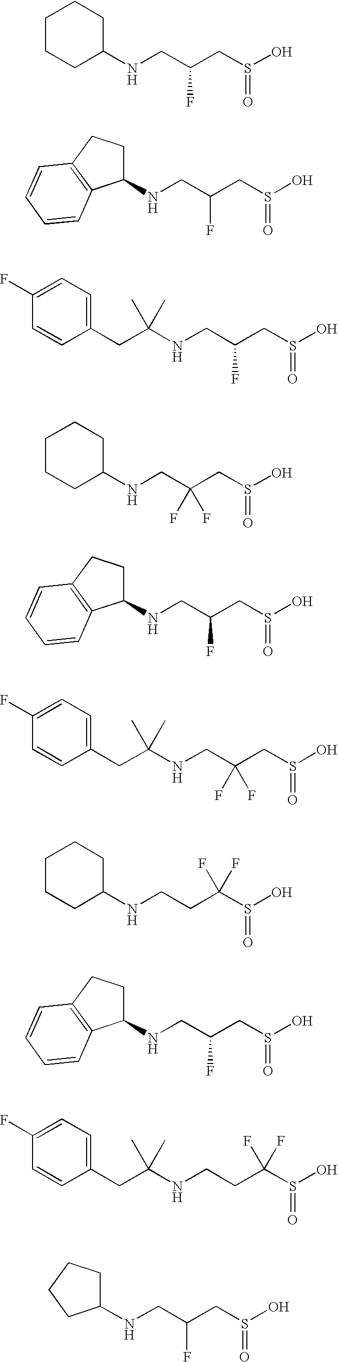 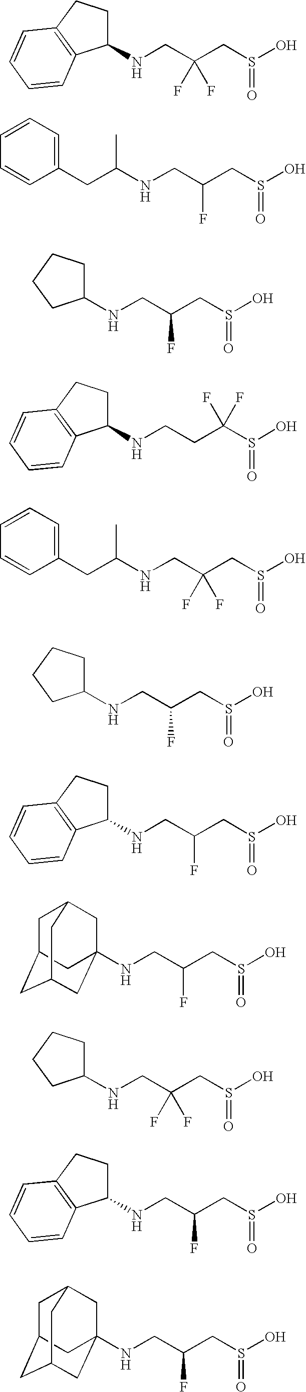 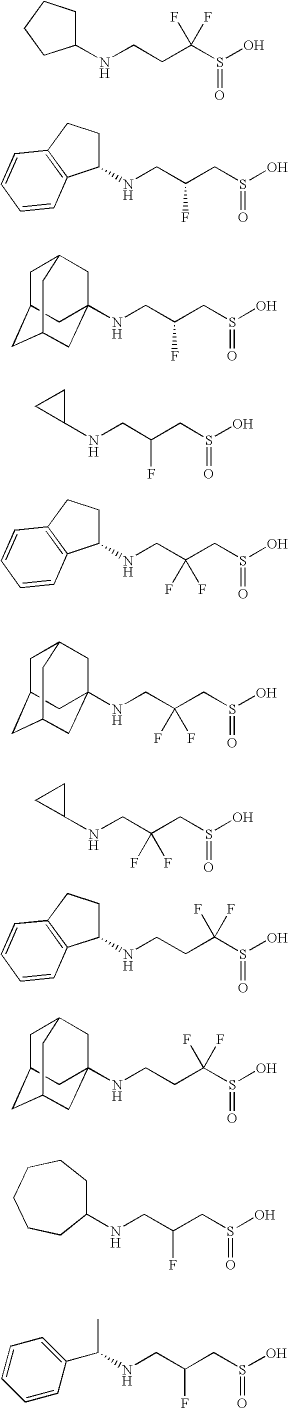 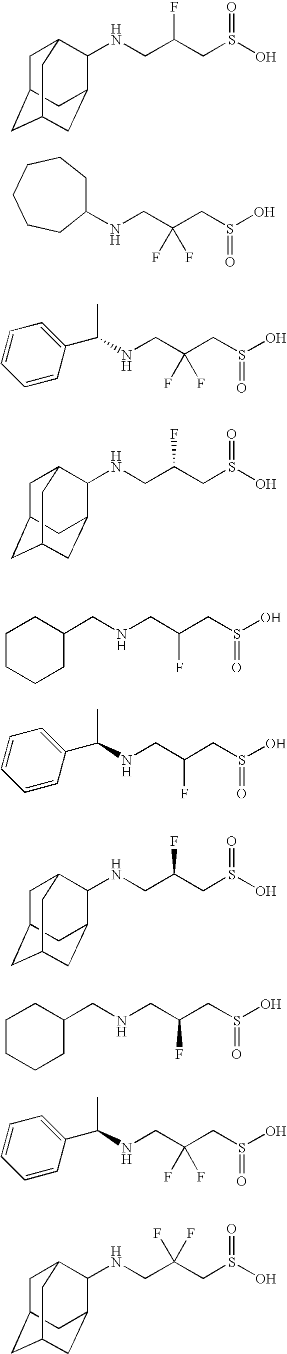 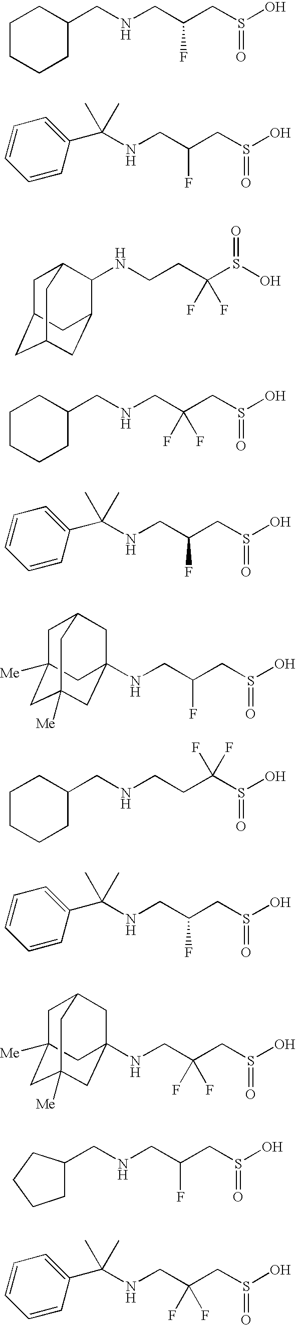 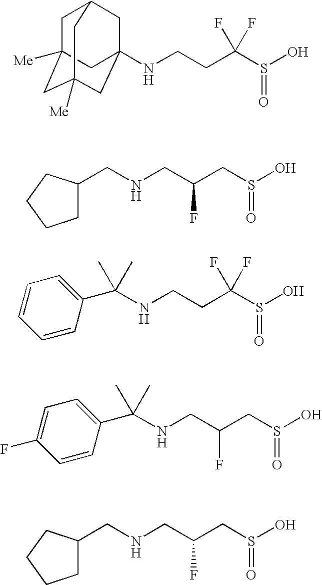
and pharmaceutically acceptable salts, esters, or prodrugs thereof.

In another embodiment, wherein L¹ is a substituted or unsubstituted alkyl group, R² is a hydrogen, L² is a propyl group, and Y is SO₃⁻H, R¹ is not a substituted phenyl group. In another embodiment, wherein R¹ is a substituted phenyl, L² is (CH₂)₃, and Y is SO₃H, then L¹ is not substituted with a cyclohexyl or cyclopentyl group. In yet another embodiment, wherein L² is (CH₂)₃, Y is SO₃H, L¹ is not an alkynyl group.

In one embodiment, the compounds of formula (I) include the compounds of formula (II): 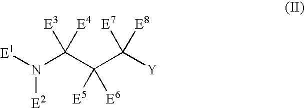
wherein:

E¹ and E² are each independently hydrogen or fluorine;

E³, E⁴, E⁵, E⁶, E⁷, and E⁸ are each independently fluorine, hydrogen, a substituted or unsubstituted cycloalkyl, a substituted or unsubstituted aryl, a substituted or unsubstituted acyl, a substituted or unsubstituted arylcycloalkyl, a substituted or unsubstituted bicyclic or tricyclic ring, a bicyclic or tricyclic fused ring group, or a substituted or unsubstituted C₂-C₁₀ alkyl group;

Y is SO₃⁻X⁺, OSO₃⁻X⁺, SSO₃⁻X⁺, or SO₂⁻X⁺;

X⁺ is hydrogen or a cationic group; and pharmaceutically acceptable salts, esters, or prodrugs thereof, provided that at least one of E¹, E², E³, E⁴, E⁵, E⁶, E⁷, and E⁸ comprise at least one or more fluorine atoms.

In one embodiment, E¹ and E² are each hydrogen. In another embodiment, E⁴, E⁵, E⁶, E⁷, and E⁸ are each independently hydrogen, fluorine, alkyl (e.g., substituted or unsubstituted C₂-C₁₀ alkyl group), fused ring (e.g., adamantyl), or aryl (e.g., substituted or unsubstituted phenyl or substituted or unsubstituted heteroaryl). The substituted alkyl group may be substituted with any substituent that allows it to perform its intended function. In another embodiment, E⁴ is hydrogen.

In another embodiment, E⁵ is hydrogen, fluorine, substituted benzyl (e.g., fluorinated benzyl), or alkyl substituted with a fused ring. An example of an alkyl substituted with a fused ring includes an alkyl substituted with an adamantyl moiety which can be optionally substituted with fluorine. In yet another embodiment, E⁶ and E⁷ are each independently hydrogen or fluorine. In another embodiment, E⁸ is hydrogen, fluorine, or alkyl substituted with a fused ring. In a further embodiment, Y is SO₃⁻X⁺.

In another embodiment, E³ is hydrogen, substituted or unsubstituted alkyl, substituted or unsubstituted cycloalkyl (e.g., substituted or unsubstituted cyclopropyl, cyclopentyl, cyclohexyl, cycloheptyl, cyclooctyl, etc.), or substituted or unsubstituted phenyl. Examples of unsubstituted alkyls include methyl, ethyl, propyl, butyl, pentyl, and hexyl. Further examples of unsubstituted alkyls include but are not limited to —CH₂CH(CH₃)₂. An example of a substituted phenyl includes fluorinated phenyl. In yet another embodiment, E³ is alkyl substituted with a fused ring. An example of a fused ring included in the invention is adamantyl, which can optionally be substituted with one or more fluorines.

The structures of some of the compounds of this invention include stereogenic carbon atoms. It is to be understood that isomers arising from such asymmetry (e.g., all enantiomers and diastereomers) are included within the scope of this invention unless indicated otherwise.

In another embodiment, the carbon to which E³ and E⁴ are attached has R stereochemistry. In another embodiment, the carbon to which E³ and E⁴ are attached has S stereochemistry. In another embodimenet, the carbon to which E⁵ and E⁶ are attached has R stereochemistry. In still another embodiment, the carbon to which E⁵ and E⁶ are attached has S stereochemistry. In yet another embodiment, the carbon to which E⁷ and E⁸ are attached has R stereochemistry. In another embodiment, the carbon to which E⁷ and E⁸ are attached has S stereochemistry. In a further embodiment, the compounds of the invention include racemic mixtures.

In another embodiment, the compound is selected from the group consisting of: 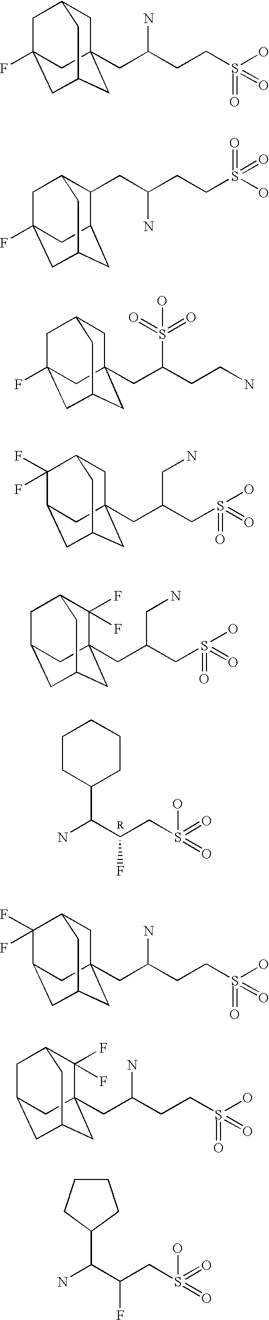 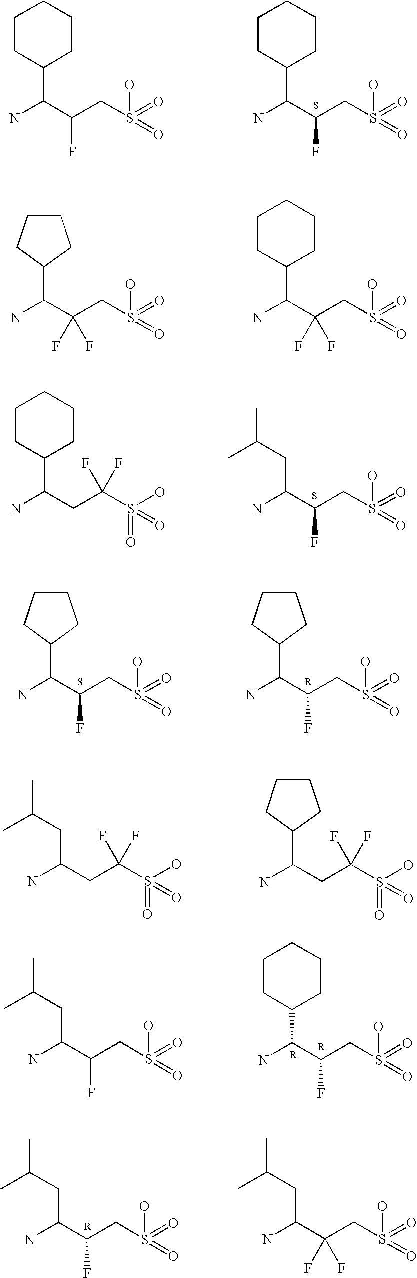 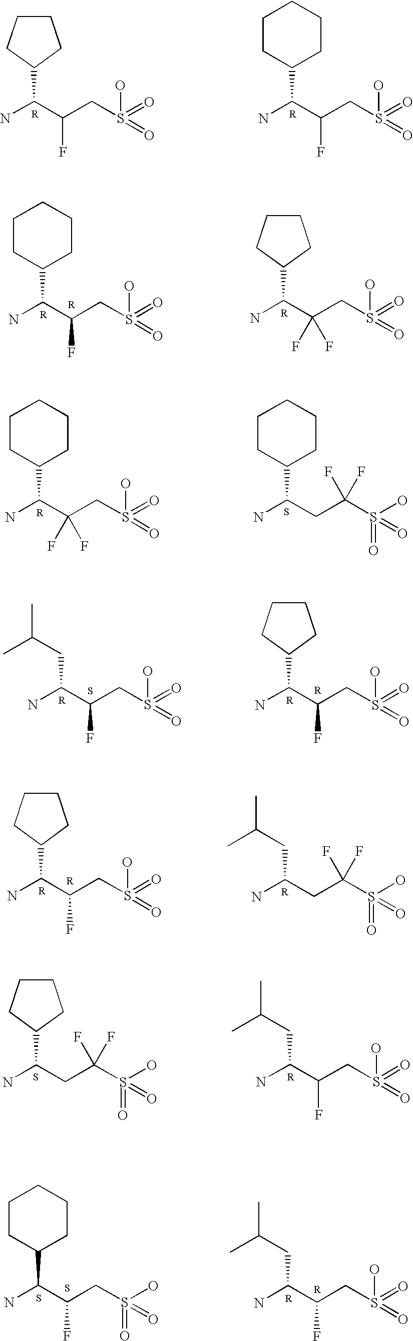 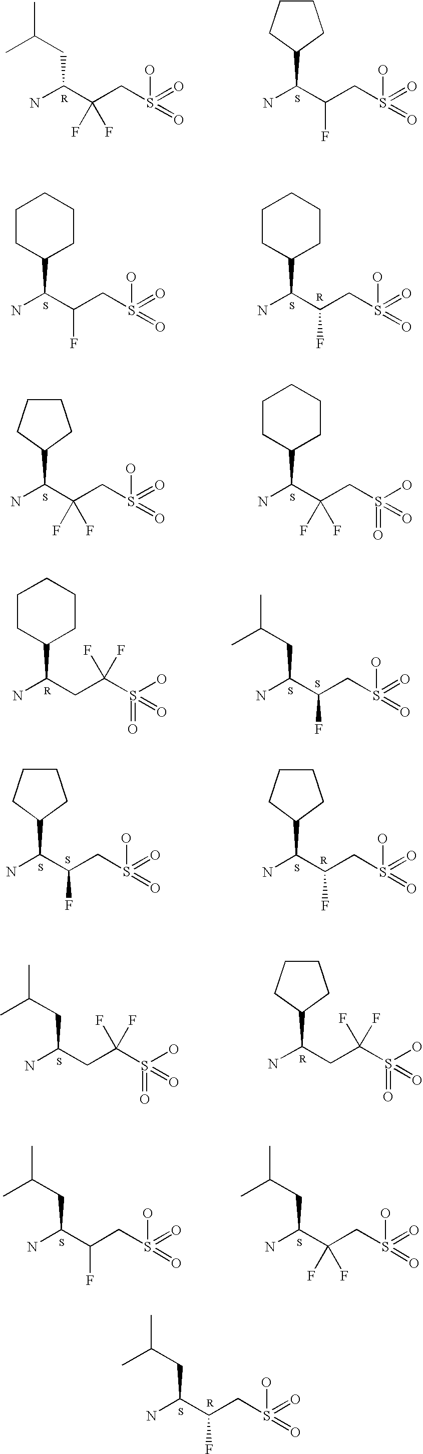 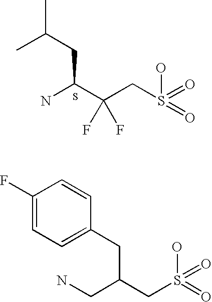
and pharmaceutically acceptable salts, esters, or prodrugs thereof.

One of skill in the art will appreciate that the nitrogen groups of the compounds of the invention are hydrogenated as necessary.

In another embodiment, the compounds of the invention include, but are not limited to, 3-amino-2-fluoro-1-propanesulfonic acid; 2-(S)-3-amino-2-fluoro-1-propanesulfonic acid; 2-(R)-3-amino-2-fluoro-1-propanesulfonic acid; 3-amino-2,2-difluoro-1-propanesulfonic acid; 3-amino-1,1-difluoro-1-propanesulfonic acid; 3-amino-1,1,2,2-tetrafluoro-1-propanesulfonic acid; 3-amino-1,1,2,2,3,3-hexafluoro-1-propanesulfonic acid; 3-t-butylamino-2-fluoro-1-propanesulfonic acid; 2-(S)-3-t-butylamino-2-fluoro-1-propanesulfonic acid; 2-(R)-3-t-butylamino-2-fluoro-1-propanesulfonic acid; 3-t-butylamino-2,2-difluoro-1-propanesulfonic acid; 3-t-butylamino-1,1-difluoro-1-propanesulfonic acid; 3-cyclohexylamino-2-fluoro-1-propanesulfonic acid; 2-(S)-3-cyclohexylamino-2-fluoro-1-propanesulfonic acid; 2-(R)-3-cyclohexylamino-2-fluoro-1-propanesulfonic acid; 3-cyclohexylamino-2,2-difluoro-1-propanesulfonic acid; 3-cyclopentylamino-2-fluoro-1-propanesulfonic acid; 2-(S)-3-cyclopentylamino-2-fluoro-1-propanesulfonic acid; 2-(R)-3-cyclopentylamino-2-fluoro-1-propanesulfonic acid; 3-cyclopentylamino-2,2-difluoro-1-propanesulfonic acid; 3-cyclopropylamino-2-fluoro-1-propanesulfonic acid; 2-(S)-3-cyclopropylamino-2-fluoro-1-propanesulfonic acid; 2-(R)-3-cyclopropylamino-2-fluoro-1-propanesulfonic acid; 3-cyclopropylamino-2,2-difluoro-1-propanesulfonic acid; 3-cycloheptylamino-2-fluoro-1-propanesulfonic acid; 2-(S)-3-cycloheptylamino-2-fluoro-1-propanesulfonic acid; 2-(R)-3-cycloheptylamino-2-fluoro-1-propanesulfonic acid; 3-cycloheptylamino-2,2-difluoro-1-propanesulfonic acid; 3-cyclohexylmethylamino-2-fluoro-1-propanesulfonic acid; 2-(S)-3-cyclohexylmethylamino-2-fluoro-1-propanesulfonic acid; 2-(R)-3-cyclohexylmethylamino-2-fluoro-1-propanesulfonic acid; 3-cyclohexylmethylamino-2,2-difluoro-1-propanesulfonic acid; 3-cyclohexylmethylamino-1,1-difluoro-1-propanesulfonic acid; 3-cyclopentylmethylamino-2-fluoro-1-propanesulfonic acid; 2-(S)-3-cyclopentylmethylamino-2-fluoro-1-propanesulfonic acid; 2-(R)-3-cyclopentylmethylamino-2-fluoro-1-propanesulfonic acid; 3-cyclopentylmethylamino-2,2-difluoro-1-propanesulfonic acid; 3-cyclopropylmethylamino-2-fluoro-1-propanesulfonic acid; 2-(S)-3-cyclopropylmethylamino-2-fluoro-1-propanesulfonic acid; 2-(R)-3-cyclopropylmethylamino-2-fluoro-1-propanesulfonic acid; 3-cyclopropylmethylamino-2,2-difluoro-1-propanesulfonic acid; 2-fluoro-3-(1,2,3,4-tetrahydro-2-naphthylamine)-1-propanesulfonic acid; 2-(S)-2-fluoro-3-(1,2,3,4-tetrahydro-2-naphthylamine)-1-propanesulfonic acid; 2-(R)-2-fluoro-3-(1,2,3,4-tetrahydro-2-naphthylamine)-1-propanesulfonic acid; 2,2-difluoro-3-(1,2,3,4-tetrahydro-2-naphthylamine)-1-propanesulfonic acid; 2-fluoro-3-(1-indanamino)-1-propanesulfonic acid; 2-(S)-2-fluoro-3-(1-indanamino)-1-propanesulfonic acid; 2-(R)-2-fluoro-3-(1-indanamino)-1-propanesulfonic acid; 2,2-difluoro-3-(1-indanamino)-1-propanesulfonic acid; 1,1-difluoro-3-(1-indanamino)-1-propanesulfonic acid; 1′-(R)-2-fluoro-3-(1-indanamino)-1-propanesulfonic acid; (1′R; 2S)-2-fluoro-3-(1-indanamino)-1-propanesulfonic acid; (1′R; 2R)-2-fluoro-3-(1-indanamino)-1-propanesulfonic acid; 1′-(R)-2,2-difluoro-3-(1-indanamino)-1-propanesulfonic acid; 1′-(S)-2-fluoro-3-(1-indanamino)-1-propanesulfonic acid; (1′S; 2S)-2-fluoro-3-(1-indanamino)-1-propanesulfonic acid; (1′S; 2R)-2-fluoro-3-(1-indanamino)-1-propanesulfonic acid; 1′-(S)-2,2-difluoro-3-(1-indanamino)-1-propanesulfonic acid; 1′-(S)-2-fluoro-3-(1-methylbenzylamino)-1-propanesulfonic acid; (1′S, 2S)-2-fluoro-3-(1-methylbenzylamino)-1-propanesulfonic acid; (1′S, 2R)-2-fluoro-3-(1-methylbenzylamino)-1-propanesulfonic acid; 1′-(S)-2,2-difluoro-3-(1-methylbenzylamino)-1-propanesulfonic acid; 1′-(R)-2-fluoro-3-(1-methylbenzylamino)-1-propanesulfonic acid; (1′R; 2S)-2-fluoro-3-(1-methylbenzylamino)-1-propanesulfonic acid; (1′R; 2R)-2-fluoro-3-(1-methylbenzylamino)-1-propanesulfonic acid; 1′-(R)-2,2-difluoro-3-(1-methylbenzylamino)-1-propanesulfonic acid; 1′-(S)-2-fluoro-3-(1-ethylbenzylamino)-1-propanesulfonic acid; (1′S; 2S)-2-fluoro-3-(1-ethylbenzylamino)-1-propanesulfonic acid; (1′S, 2R)-2-fluoro-3-(1-ethylbenzylamino)-1-propanesulfonic acid; 1′-(S)-2,2-difluoro-3-(1-ethylbenzylamino)-1-propanesulfonic acid; 1′-(R)-2-fluoro-3-(1-ethylbenzylamino)-1-propanesulfonic acid; (1′R; 2S)-2-fluoro-3-(1-ethylbenzylamino)-1-propanesulfonic acid; (1′R; 2R)-2-fluoro-3-(1-ethylbenzylamino)-1-propanesulfonic acid; 1′-(R)-2,2-difluoro-3-(1-ethylbenzylamino)-1-propanesulfonic acid; 3-[1,1-dimethyl-1-benzylamino)-2-fluoro-1-propanesulfonic acid; 2-(S)-3-(1,1-dimethyl-1-benzylamino)-2-fluoro-1-propanesulfonic acid; 2-(R)-3-(1,1-dimethyl-1-benzylamino)-2-fluoro-1-propanesulfonic acid; 2,2-difluoro-3-(1,1-dimethyl-1-benzylamino)-1-propanesulfonic acid; 3-[1,1-dimethyl-1-(4-fluorobenzyl)amino]-2-fluoro-1-propanesulfonic acid; 2-(S)-3-[(1,1-dimethyl-1-(4-fluorobenzyl)amino]-2-fluoro-1-propanesulfonic acid; 2-(R)-3-[(1,1-dimethyl-1-(4-fluorobenzyl)amino]-2-fluoro-1-propanesulfonic acid; 2,2-difluoro-3-[(1,1-dimethyl-1-(4-fluorobenzyl)amino]-1-propanesulfonic acid; 2-fluoro-3-[2-methyl-1-(4-methylphenyl)-2-propylamino]-1-propanesulfonic acid; 2-(S)-2-fluoro-3-[2-methyl-1-(4-methylphenyl)-2-propylamino]-1-propanesulfonic acid; 2-(R)-2-fluoro-3-[2-methyl-1-(4-methylphenyl)-2-propylamino]-1-propanesulfonic acid; 2,2-difluoro-3-[2-methyl-1-(4-methylphenyl)-2-propylamino]-1-propanesulfonic acid; 2-fluoro-3-[1-(4-fluorophenyl)-2-methyl-2-propylamino]-1-propanesulfonic acid; 2-(S)-2-fluoro-3-[1-(4-flurophenyl)-2-methyl-2-propylamino]-1-propanesulfonic acid; 2-(R)-2-fluoro-3-[1-(4-flurophenyl)-2-methyl-2-propylamino]-1-propanesulfonic acid; 2,2-difluoro-3-[1-(4-flurophenyl)-2-methyl-2-propylamino]-1-propanesulfonic acid; 2-fluoro-3-(1-phenyl-2-propylamino)-1-propanesulfonic acid; 2-(S)-2-fluoro-3-(1-phenyl-2-propylamino)-1-propanesulfonic acid; 2-(R)-2-fluoro-3-(1-phenyl-2-propylamino)-1-propanesulfonic acid; 2,2-difluoro-3-(1-phenyl-2-propylamino)-1-propanesulfonic acid; 3-(1-adamantanamino)-2-fluoro-1-propanesulfonic acid; 2-(S)-3-(1-adamantanamino)-2-fluoro-1-propanesulfonic acid; 2-(R)-3-(1-adamantanamino)-2-fluoro-1-propanesulfonic acid; 3-(1-adamantanamino)-2,2-difluoro-1-propanesulfonic acid; 3-(2-adamantanamino)-2-fluoro-1-propanesulfonic acid; 2-(S)-3-(2-adamantanamino)-2-fluoro-1-propanesulfonic acid; 2-(R)-3-(2-adamantanamino)-2-fluoro-1-propanesulfonic acid; 3-(2-adamantanamino)-2,2-difluoro-1-propanesulfonic acid; 3-(3,5-dimethyl-1-adamantanamino)-2-fluoro-1-propanesulfonic acid; 2-(S)-3-(3,5-dimethyl-1-adamantanamino)-2-fluoro-1-propanesulfonic acid; 2-(R)-3-(3,5-dimethyl-1-adamantanamino)-2-fluoro-1-propanesulfonic acid; 3-(3,5-dimethyl-1-adamantanamino)-2,2-difluoro-1-propanesulfonic acid; 3-amino-2,2-difluoro-1-propanesulfinic acid; 3-amino-1,1-difluoro-1-propanesulfinic acid; 3-amino-1,1,2,2-tetrafluoro-1-propanesulfinic acid; 3-amino-1,1,2,2,3,3-hexafluoro-1-propanesulfinic acid; 3-t-butylamino-2-fluoro-1-propanesulfinic acid; 2-(S)-3-t-butylamino-2-fluoro-1-propanesulfinic acid; 2-(R)-3-t-butylamino-2-fluoro-1-propanesulfinic acid; 3-t-butylamino-2,2-difluoro-1-propanesulfinic acid; 3-t-butylamino-1,1-difluoro-1-propanesulfinic acid; 3-cyclohexylamino-2-fluoro-1-propanesulfinic acid; 2-(S)-3-cyclohexylamino-2-fluoro-1-propanesulfinic acid; 2-(R)-3-cyclohexylamino-2-fluoro-1-propanesulfinic acid; 3-cyclohexylamino-2,2-difluoro-1-propanesulfinic acid; 3-cyclohexylamino-1,1-difluoro-1-propanesulfinic acid; 3-cyclopentylamino-2-fluoro-1-propanesulfinic acid; 2-(S)-3-cyclopentylamino-2-fluoro-1-propanesulfinic acid; 2-(R)-3-cyclopentylamino-2-fluoro-1-propanesulfinic acid; 3-cyclopentylamino-2,2-difluoro-1-propanesulfinic acid; 3-cyclopentylamino-1,1-difluoro-1-propanesulfinic acid; 3-cyclopropylamino-2-fluoro-1-propanesulfinic acid; 3-cyclopropylamino-2,2-difluoro-1-propanesulfinic acid; 3-cycloheptylamino-2-fluoro-1-propanesulfinic acid; 3-cycloheptylamino-2,2-difluoro-1-propanesulfinic acid; 3-cyclohexylmethylamino-2-fluoro-1-propanesulfinic acid; 2-(S)-3-cyclohexylmethylamino-2-fluoro-1-propanesulfinic acid; 2-(R)-3-cyclohexylmethylamino-2-fluoro-1-propanesulfinic acid; 3-cyclohexylmethylamino-2,2-difluoro-1-propanesulfinic acid; 3-cyclohexylmethylamino-1,1-difluoro-1-propanesulfinic acid; 3-cyclopentylmethylamino-2-fluoro-1-propanesulfinic acid; 2-(S)-3-cyclopentylmethylamino-2-fluoro-1-propanesulfinic acid; 2-(R)-3-cyclopentylmethylamino-2-fluoro-1-propanesulfinic acid; 3-cyclopentylmethylamino-2,2-difluoro-1-propanesulfinic acid; 3-cyclopentylmethylamino-1,1-difluoro-1-propanesulfinic acid; 3-cyclopropylmethylamino-2-fluoro-1-propanesulfinic acid; 3-cyclopropylmethylamino-2,2-difluoro-1-propanesulfinic acid; 2-fluoro-3-(1,2,3,4-tetrahydro-2-naphthylamine)-1-propanesulfinic acid; 2,2-difluoro-3-(1,2,3,4-tetrahydro-2-naphthylamine)-1-propanesulfinic acid; 2-fluoro-3-(1-indanamino)-1-propanesulfinic acid; 2-(s)-2-fluoro-3-(1-indanamino)-1-propanesulfinic acid; 2-(R)-2-fluoro-3-(1-indanamino)-1-propanesulfinic acid; 2,2-difluoro-3-(1-indanamino)-1-propanesulfinic acid; 1,1-difluoro-3-(1-indanamino)-1-propanesulfinic acid; 1′-(R)-2-fluoro-3-(1-indanamino)-1-propanesulfinic acid; (1′R; 2S)-2-fluoro-3-(1-indanamino)-1-propanesulfinic acid; (1′R; 2R)-2-fluoro-3-(1-indanamino)-1-propanesulfinic acid; 1′-(R)-2,2-difluoro-3-(1-indanamino)-1-propanesulfinic acid; 1′-(R)-1,1-difluoro-3-(1-indanamino)-1-propanesulfinic acid; 1′-(s)-2-fluoro-3-(1-indanamino)-1-propanesulfinic acid; (1′S, 2S)-2-fluoro-3-(1-indanamino)-1-propanesulfinic acid; (1′S, 2R)-2-fluoro-3-(1-indanamino)-1-propanesulfinic acid; 1′-(S)-2,2-difluoro-3-(1-indanamino)-1-propanesulfinic acid; 1′-(S)-1,1-difluoro-3-(1-indanamino)-1-propanesulfinic acid; 1′-(S)-2-fluoro-3-(1-methylbenzylamino)-1-propanesulfinic acid; 1′-(S)-2,2-difluoro-3-(1-methylbenzylamino)-1-propanesulfinic acid; 1′-(R)-2-fluoro-3-(1-methylbenzylamino)-1-propanesulfinic acid; 1′-(R)-2,2-difluoro-3-(1-methylbenzylamino)-1-propanesulfinic acid; 3-[1,1-dimethyl-1-benzylamino)-2-fluoro-1-propanesulfinic acid; 2-(S)-3-(1,1-dimethyl-1-benzylamino)-2-fluoro-1-propanesulfinic acid; 2-(R)-3-(1,1-dimethyl-1-benzylamino)-2-fluoro-1-propanesulfinic acid; 2,2-difluoro-3-(1,1-dimethyl-1-benzylamino)-1-propanesulfinic acid; 2,2-difluoro-3-(1,1-dimethyl-1-benzylamino)-1-propanesulfinic acid; 3-[1,1-dimethyl-1-(4-fluorobenzyl)amino]-2-fluoro-1-propanesulfinic acid; 2-(S)-3-[(1,1-dimethyl-1-(4-fluorobenzyl)amino]-2-fluoro-1-propanesulfinic acid; 2-(R)-3-[(1,1-dimethyl-1-(4-fluorobenzyl)amino]-2-fluoro-1-propanesulfinic acid; 2,2-difluoro-3-[(1,1-dimethyl-1-(4-fluorobenzyl)amino]-1-propanesulfinic acid; 1,1-difluoro-3-[(1,1-dimethyl-1-(4-fluorobenzyl)amino]-1-propanesulfinic acid; 2-fluoro-3-[2-methyl-1-(4-methylphenyl)-2-propylamino]-1-propanesulfinic acid; 2-(S)-2-fluoro-3-[2-methyl-1-(4-methylphenyl)-2-propylamino]-1-propanesulfinic acid; 2-(R)-2-fluoro-3-[2-methyl-1-(4-methylphenyl)-2-propylamino]-1-propanesulfinic acid; 2,2-difluoro-3-[2-methyl-1-(4-methylphenyl)-2-propylamino]-1-propanesulfinic acid; 1,1-difluoro-3-[2-methyl-1-(4-methylphenyl)-2-propylamino]-1-propanesulfinic acid; 2-fluoro-3-[1-(4-fluorophenyl)-2-methyl-2-propylamino]-1-propanesulfinic acid; 2-(S)-2-fluoro-3-[1-(4-flurophenyl)-2-methyl-2-propylamino]-1-propanesulfinic acid; 2-(R)-2-fluoro-3-[1-(4-flurophenyl)-2-methyl-2-propylamino]-1-propanesulfinic acid; 2,2-difluoro-3-[1-(4-flurophenyl)-2-methyl-2-propylamino]-1-propanesulfinic acid; 1,1-difluoro-3-[1-(4-flurophenyl)-2-methyl-2-propylamino]-1-propanesulfinic acid; 2-fluoro-3-(1-phenyl-2-propylamino)-1-propanesulfinic acid; 2,2-difluoro-3-(1-phenyl-2-propylamino)-1-propanesulfinic acid; 3-(1-adamantanamino)-2-fluoro-1-propanesulfinic acid; 2-(S)-3-(1-adamantanamino)-2-fluoro-1-propanesulfinic acid; 2-(R)-3-(1-adamantanamino)-2-fluoro-1-propanesulfinic acid; 3-(1-adamantanamino)-2,2-difluoro-1-propanesulfinic acid; 3-(1-adamantanamino)-1,1-difluoro-1-propanesulfinic acid; 3-(2-adamantanamino)-2-fluoro-1-propanesulfinic acid; 2-(S)-3-(2-adamantanamino)-2-fluoro-1-propanesulfinic acid; 2-(R)-3-(2-adamantanamino)-2-fluoro-1-propanesulfinic acid; 3-(2-adamantanamino)-2,2-difluoro-1-propanesulfinic acid; 3-(2-adamantanamino)-1,1-difluoro-1-propanesulfinic acid; 3-(3,5-dimethyl-1-adamantanamino)-2-fluoro-1-propanesulfinic acid; 3-(3,5-dimethyl-1-adamantanamino)-2,2-difluoro-1-propanesulfinic acid; 3-(3,5-dimethyl-1-adamantanamino)-1,1-difluoro-1-propanesulfinic acid; and pharmaceutically acceptable salts, esters, or prodrugs thereof.

In one embodiment, the compounds of the invention do not include 3-amino-2-fluoro-1-propanesulfinic acid; 2-(S)-3-amino-2-fluoro-1-propanesulfonic acid; or 2-(R)-3-amino-2-fluoro-1-propanesulfonic acid.

In one embodiment, when L¹ is a carbonyl, R¹ is not CₚH_{q}Fᵣ—CₓH_{y}, wherein p is an integer from 1 to 20; q is an integer from 1 to 40; r is an integer from 1 to 40, x is an integer from 0 to 25; and y is an integer from 0 to 50. In another embodiment, when L¹ is a carbonyl, R¹ is not CₚH_{q}Fᵣ—CₓH_{y}, wherein CₚH_{q}Fᵣ is an aryl or alkylaryl group. In yet another embodiment, when L¹ is carbonyl, R¹ is not CₚH_{q}Fᵣ—CₓH_{y}, wherein CₚH_{q}Fᵣ is a phenyl moiety with at least one perfluoro-1H-1H neopentyl substituent.

In one embodiment, when L¹ is a carbonyl, R¹ is not CₚFᵣ—CₓH_{y}, wherein p is an integer from 1 to 20; r is an integer from 3 to 41; x is an integer from 0 to 25; and y is an integer from 0 to 50.

In another embodiment, when L¹ is carbonyl, R¹ is not CF₃—(CH₂)ₓ_{₁}, wherein x₁ is an integer from 0 to 25. In yet another embodiment, when L¹ is carbonyl, R¹ is not (CF₃)₃C—(CH₂)ₓ_{₂}, wherein x₂ is an integer from 1 to 25.

In yet another embodiment, L¹ (or R¹ if L¹ is absent), is not an acyl group. In yet another embodiment, L¹ (or R¹ if L¹ is absent), is an acyl group.

In one embodiment, the invention does not pertain to the compounds described in WO 00/64420, WO 96/28187, WO 02/100823, U.S. Pat. No. 5,660,815, and/or U.S. Pat. No. 6,451,761. In this embodiment, the invention does not pertain to methods of using the compounds described in WO 00/64420, WO 96/28187, WO 02/100823 U.S. Pat. No. 5,660,815 and/or U.S. Pat. No. 6,451,761 for the treatment of diseases or disorders described therein. Each of WO 00/64420, WO 96/28187, WO 02/100823 U.S. Pat. No. 5,660,815 and U.S. Pat. No. 6,451,761 are incorporated by reference herein in their entirety.

In another embodiment, the invention pertains to the fluorinated compounds described in U.S. patent application Ser. No. 10/871,514, filed Jun. 18, 2004, which is incorporated herein by reference in its entirety.

It should be understood that the use of any of the compounds described herein is within the scope of the present invention and is intended to be encompassed by the present invention and each of the applications and patents listed above or elsewhere in the application are expressly incorporated herein at least for these purposes, and are furthermore expressly incorporated for all other purposes.

Subjects and Patient Populations

The term “subject” includes living organisms in which amyloidosis can occur, or which are susceptible to amyloid diseases, e.g., Alzheimer's disease, Down's syndrome, CAA, dialysis-related (β₂M) amyloidosis, secondary (AA) amyloidosis, primary (AL) amyloidosis, hereditary amyloidosis, diabetes, etc. Examples of subjects include humans, chickens, ducks, peking ducks, geese, monkeys, deer, cows, rabbits, sheep, goats, dogs, cats, mice, rats, and transgenic species thereof. Administration of the compositions of the present invention to a subject to be treated can be carried out using known procedures, at dosages and for periods of time effective to modulate amyloid aggregation or amyloid-induced toxicity in the subject as further described herein. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to modulate amyloid aggregation in the subject. Dosage regimens can be adjusted to provide the optimum therapeutic response. For example, several divided doses may be administered daily or the dose may be proportionally reduced as indicated by the exigencies of the therapeutic situation.

In certain embodiments of the invention, the subject is in need of treatment by the methods of the invention, and is selected for treatment based on this need. A subject in need of treatment is art-recognized, and includes subjects that have been identified as having a disease or disorder related to amyloid-deposition or amyloidosis, have a symptom of such a disease or disorder, or are at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

In an exemplary aspect of the invention, the subject is a human. For example, the subject may be a human over 30 years old, human over 40 years old, a human over 50 years old, a human over 60 years old, a human over 70 years old, a human over 80 years old, a human over 85 years old, a human over 90 years old, or a human over 95 years old. The subject may be a female human, including a postmenopausal female human, who may be on hormone (estrogen) replacement therapy. The subject may also be a male human. In another embodiment, the subject is under 40 years old.

A subject may be a human at risk for Alzheimer's disease, e.g., being over the age of 40 or having a predisposition for Alzheimer's disease. Alzheimer's disease predisposing factors identified or proposed in the scientific literature include, among others, a genotype predisposing a subject to Alzheimer's disease; environmental factors predisposing a subject to Alzheimer's disease; past history of infection by viral and bacterial agents predisposing a subject to Alzheimer's disease; and vascular factors predisposing a subject to Alzheimer's disease. A subject may also have one or more risk factors for cardiovascular disease (e.g., atherosclerosis of the coronary arteries, angina pectoris, and myocardial infarction) or cerebrovascular disease (e.g., atherosclerosis of the intracranial or extracranial arteries, stroke, syncope, and transient ischemic attacks), such as hypercholesterolemia, hypertension, diabetes, cigarette smoking, familial or previous history of coronary artery disease, cerebrovascular disease, and cardiovascular disease. Hypercholesterolemia typically is defined as a serum total cholesterol concentration of greater than about 5.2 mmol/L (about 200 mg/dL).

Several genotypes are believed to predispose a subject to Alzheimer's disease. These include the genotypes such as presenilin-1, presenilin-2, and amyloid precursor protein (APP) missense mutations associated with familial Alzheimer's disease, and α-2-macroglobulin and LRP-1 genotypes, which are thought to increase the risk of acquiring sporadic (late-onset) Alzheimer's disease. E. van Uden, et al., J. Neurosci. 22(21), 9298-304 (2002); J. J. Goto, et al., J. Mol. Neurosci. 19(1-2), 37-41 (2002). Another genetic risk factor for the development of Alzheimer's disease are variants of ApoE, the gene that encodes apolipoprotein E (particularly the apoE4 genotype), a constituent of the low-density lipoprotein particle. W J Strittmatter, et al., Annu. Rev. Neurosci. 19, 53-77 (1996). The molecular mechanisms by which the various ApoE alleles alter the likelihood of developing Alzheimer's disease are unknown, however the role of ApoE in cholesterol metabolism is consistent with the growing body of evidence linking cholesterol metabolism to Alzheimer's disease. For example, chronic use of cholesterol-lowering drugs such as statins has recently been associated with a lower incidence of Alzheimer's disease, and cholesterol-lowering drugs have been shown to reduce pathology in APP transgenic mice. These and other studies suggest that cholesterol may affect APP processing. ApoE4 has been suggested to alter Aβ trafficking (in and out of the brain), and favor retention of Aβ in the brain. ApoE4 has also been suggested to favor APP processing toward Aβ formation. Environmental factors have been proposed as predisposing a subject to Alzheimer's disease, including exposure to aluminum, although the epidemiological evidence is ambiguous. In addition, prior infection by certain viral or bacterial agents may predispose a subject to Alzheimer's disease, including the herpes simplex virus and chlamydia pneumoniae. Finally, other predisposing factors for Alzheimer's disease can include risk factors for cardiovascular or cerebrovascular disease, including cigarette smoking, hypertension and diabetes. “At risk for Alzheimer's disease” also encompasses any other predisposing factors not listed above or as yet identified and includes an increased risk for Alzheimer's disease caused by head injury, medications, diet, or lifestyle.

The methods of the present invention can be used for one or more of the following: to prevent Alzheimer's disease, to treat Alzheimer's disease, to ameliorate symptoms of Alzheimer's disease, or to regulate production of or levels of amyloid β (Aβ) peptides. In an embodiment, the human carries one or more mutations in the genes that encode β-amyloid precursor protein, presenilin-1 or presenilin-2. In another embodiment, the human carries the Apolipoprotein ε4 gene. In another embodiment, the human has a family history of Alzheimer's Disease or a dementia illness. In another embodiment, the human has trisomy 21 (Down's Syndrome). In another embodiment, the subject has a normal or low serum total blood cholesterol level. In another embodiment, the serum total blood cholesterol level is less than about 200 mg/dL, or less than about 180, and it can range from about 150 to about 200 mg/dL. In another embodiment, the total LDL cholesterol level is less than about 100 mg/dL, or less than about 90 mg/dL and can range from about 30 to about 100 mg/dL. Methods of measuring serum total blood cholesterol and total LDL cholesterol are well known to those skilled in the art and for example include those disclosed in WO 99/38498 at p. 11, incorporated by reference herein. Methods of determining levels of other sterols in serum are disclosed in H. Gylling, et al., “Serum Sterols During Stanol Ester Feeding in a Mildly Hypercholesterolemic Population”, J. Lipid Res. 40: 593-600 (1999).

In another embodiment, the subject has an elevated serum total blood cholesterol level. In another embodiment, the serum total cholesterol level is at least about 200 mg/dL, or at least about 220 mg/dL and can range from about 200 to about 1000 mg/dL. In another embodiment, the subject has an elevated total LDL cholesterol level. In another embodiment, the total LDL cholesterol level is greater than about 100 mg/dL, or even greater than about 110 mg/dL and can range from about 100 to about 1000 mg/dL.

In another embodiment, the human is at least about 40 years of age. In another embodiment, the human is at least about 60 years of age. In another embodiment, the human is at least about 70 years of age. In another embodiment, the human is at least about 80 years of age. In another embodiment, the human is at least about 85 years of age. In one embodiment, the human is between about 60 and about 100 years of age.

In still a further embodiment, the subject is shown to be at risk by a diagnostic brain imaging technique, for example, one that measures brain activity, plaque deposition, or brain atrophy.

In still a further embodiment, the subject is shown to be at risk by a cognitive test such as Clinical Dementia Rating (“CDR”), Alzheimer's Disease Assessment Scale-Cognitive Subscale (“ADAS-Cog”), Disability Assessment for Dementia (“DAD”), or Mini-Mental State Examination (“MMSE”). The subject may exhibit a below average score on a cognitive test, as compared to a historical control of similar age and educational background. The subject may also exhibit a reduction in score as compared to previous scores of the subject on the same or similar cognition tests.

In determining the CDR, a subject is typically assessed and rated in each of six cognitive and behavioral categories: memory, orientation, judgment and problem solving, community affairs, home and hobbies, and personal care. The assessment may include historical information provided by the subject, or preferably, a corroborator who knows the subject well. The subject is assessed and rated in each of these areas and the overall rating, (0, 0.5, 1.0, 2.0 or 3.0) determined. A rating of 0 is considered normal. A rating of 1.0 is considered to correspond to mild dementia. A subject with a CDR of 0.5 is characterized by mild consistent forgetfulness, partial recollection of events and “benign” forgetfulness. In one embodiment the subject is assessed with a rating on the CDR of above 0, of above about 0.5, of above about 1.0, of above about 1.5, of above about 2.0, of above about 2.5, or at about 3.0.

Another test is the Mini-Mental State Examination (MMSE), as described by Folstein “Mini-mental state. A practical method for grading the cognitive state of patients for the clinician.” J. Psychiatr. Res. 12:189-198, 1975. The MMSE evaluates the presence of global intellectual deterioration. See also Folstein “Differential diagnosis of dementia. The clinical process.” Psychiatr Clin North Am. 20:45-57, 1997. The MMSE is a means to evaluate the onset of dementia and the presence of global intellectual deterioration, as seen in Alzheimer's disease and multi-infart dementia. The MMSE is scored from 1 to 30. The MMSE does not evaluate basic cognitive potential, as, for example, the so-called IQ test. Instead, it tests intellectual skills. A person of “normal” intellectual capabilities will score a “30” on the MMSE objective test (however, a person with a MMSE score of 30 could also score well below “normal” on an IQ test). See, e.g., Kaufer, J. Neuropsychiatry Clin. Neurosci. 10:55-63, 1998; Becke, Alzheimer Dis Assoc Disord. 12:54-57, 1998; Ellis, Arch. Neurol. 55:360-365, 1998; Magni, Int. Psychogeriatr. 8:127-134, 1996; Monsch, Acta Neurol. Scand. 92:145-150, 1995. In one embodiment, the subject scores below 30 at least once on the MMSE. In another embodiment, the subject scores below about 28, below about 26, below about 24, below about 22, below about 20, below about 18, below about 16, below about 14, below about 12, below about 10, below about 8, below about 6, below about 4, below about 2, or below about 1.

The Disability Assessment for Dementia (“DAD”) scale has been developed to measure a patient's ability to perform the activities of daily living (Gélinas I et al. Development of a Functional Measure for Persons with Alzheimer's Disease: The Disability Assessment for Dementia Am. J. Occupational Therapy. 1999; 53: 471-481). Activities of daily living may be assessed according to self care (i.e., dressing and personal hygiene) and instrumental activities (e.g., housework, cooking, and using household devices). The objectives of the DAD scale include quantitatively measuring functional abilities in activities of daily living in individuals with cognitive impairments and to help delineate areas of cognitive deficits that may impair performance in activities of daily living. The DAD is administered through an interview with the caregiver. It measures actual performance in activities of daily living of the individual as observed over a 2 week period prior to the interview. The scale assesses the following domains of activities: hygiene, dressing, telephoning, continence, eating, meal preparation, outing activities, finance and correspondence, medication use, leisure and housework. A total score is obtained by adding the rating for each question and converting this total score out of 100. Higher scores represent less disability in ADL while lower scores indicate more dysfunction. In one embodiment, the subject scores below 100 at least once on the DAD. In another embodiment, the subject scores below about 95, below about 90, below about 85, below about 80, below about 75, below about 70, below about 65, below about 60, below about 55, below about 50, below about 45, below about 40, below about 30, below about 20, or below about 10.

Another means to evaluate cognition, particularly Alzheimer's disease, is the Alzheimer's Disease Assessment Scale (ADAS-Cog), or a variation termed the Standardized Alzheimer's Disease Assessment Scale (SADAS). It is commonly used as an efficacy measure in clinical drug trials of Alzheimer's disease and related disorders characterized by cognitive decline. SADAS and ADAS-Cog were not designed to diagnose Alzheimer's disease; they are useful in characterizing symptoms of dementia and are a relatively sensitive indicator of dementia progression. (See, e.g., Doraiswamy, Neurology 48:1511-1517, 1997; and Standish, J. Am. Geriatr. Soc. 44:712-716, 1996.) Annual deterioration in untreated Alzheimer's disease patients is approximately 8 points per year (See, e.g., Raskind, M Prim. Care Companion J Clin Psychiatry 2000 August; 2(4):134-138), but may vary according to the stage. Patients with mild cognitive disorder may have deterioration rates slower than that of patients with moderate or severe symptoms. (See, e.g., Stein et al.).

The ADAS-cog is designed to measure, with the use of questionnaires, the progression and the severity of cognitive decline as seen in AD on a 70-point scale. The ADAS-cog scale quantifies the number of wrong answers. Consequently, a high score on the scale indicates a more severe case of cognitive decline. In one embodiment, a subject exhibits a score of greater than 0, greater than about 5, greater than about 10, greater than about 15, greater than about 20, greater than about 25, greater than about 26, greater than about 30, greater than about 35, greater than about 40, greater than about 45, greater than about 50, greater than about 55, greater than about 60, greater than about 65, greater than about 68, or about 70.

In another embodiment, the subject exhibits no symptoms of Alzheimer's Disease. In another embodiment, the subject is a human who is at least 40 years of age and exhibits no symptoms of Alzheimer's Disease. In another embodiment, the subject is a human who is at least 40 years of age and exhibits one or more symptoms of Alzheimer's Disease.

In another embodiment, the subject has Mild Cognitive Impairment. In a further embodiment, the subject has a CDR rating of about 0.5. In another embodiment, the subject has early Alzheimer's disease. In another embodiment, the subject has cerebral amyloid angiopathy.

By using the methods of the present invention, the levels of amyloid β peptides in a subject's plasma or cerebrospinal fluid (CSF) can be reduced from levels prior to treatment by about 10 to about 100 percent, or even about 50 to about 100 percent.

In an alternative embodiment, the subject can have an elevated level of amyloid Aβ₄₀ and Aβ₄₂ peptide in the blood and CSF prior to treatment, according to the present methods, of greater than about 10 pg/mL, or greater than about 20 pg/mL, or greater than about 35 pg/mL, or even greater than about 40 pg/mL. In another embodiment, the elevated level of amyloid Aβ₄₂ peptide can range from about 30 pg/mL to about 200 pg/mL, or even to about 500 pg/mL. One skilled in the art would understand that as Alzheimer's disease progresses, the measurable levels of amyloid β peptide in the CSF may decrease from elevated levels present before onset of the disease. This effect is attributed to increased deposition, i.e., trapping of Aβ peptide in the brain instead of normal clearance from the brain into the CSF.

In an alternative embodiment, the subject can have an elevated level of amyloid Aβ₄₀ peptide in the blood and CSF prior to treatment, according to the present methods, of greater than about 5 pg Aβ₄₂/mL or greater than about 50 pg Aβ₄₀/mL, or greater than about 400 pg/mL. In another embodiment, the elevated level of amyloid Aβ₄₀ peptide can range from about 200 pg/mL to about 800 pg/mL, to even about 1000 pg/mL.

In another embodiment, the subject can have an elevated level of amyloid Aβ₄₂ peptide in the CSF prior to treatment, according to the present methods, of greater than about pg/mL, or greater than about 10 pg/mL, or greater than about 200 pg/mL, or greater than about 500 pg/mL. In another embodiment, the level of amyloid β peptide can range from about 10 pg/mL to about 1,000 pg/mL, or even about 100 pg/mL to about 1,000 pg/mL.

In another embodiment, the subject can have an elevated level of amyloid Aβ₄₀ peptide in the CSF prior to treatment according to the present methods of greater than about pg/mL, or greater than about 50 pg/mL, or even greater than about 100 pg/mL. In another embodiment, the level of amyloid β peptide can range from about 10 pg/mL to about 1,000 pg/mL.

The amount of amyloid β peptide in the brain, CSF, blood, or plasma of a subject can be evaluated by enzyme-linked immunosorbent assay (“ELISA”) or quantitative immunoblotting test methods or by quantitative SELDI-TOF which are well known to those skilled in the art, such as is disclosed by Zhang, et al., J. Biol. Chem. 274, 8966-72 (1999) and Zhang, et al., Biochemistry 40, 5049-55 (2001). See also, A. K. Vehmas, et al., DNA Cell Biol. 20(11), 713-21 (2001), P. Lewczuk, et al., Rapid Commun. Mass Spectrom. 17(12), 1291-96 (2003); B. M. Austen, et al., J. Peptide Sci. 6, 459-69 (2000); and H. Davies, et al., BioTechniques 27, 1258-62 (1999). These tests are performed on samples of the brain or blood which have been prepared in a manner well known to one skilled in the art. Another example of a useful method for measuring levels of amyloid β peptides is by Europium immunoassay (EIA). See, e.g., WO 99/38498 at p. 11.

The methods of the invention may be applied as a therapy for a subject having Alzheimer's disease or a dementia, or the methods of the invention may be applied as a prophylaxis against Alzheimer's disease or dementia for subject with such a predisposition, as in a subject, e.g., with a genomic mutation in the APP gene, the ApoE gene, or a presenilin gene. The subject may have (or may be predisposed to developing or may be suspected of having) vascular dementia, or senile dementia, Mild Cognitive Impairment, or early Alzheimer's disease. In addition to Alzheimer's disease, the subject may have another amyloid-related disease such as cerebral amyloid angiopathy, or the subject may have amyloid deposits, especially amyloid-β amyloid deposits in the brain.

Treatment of Amyloid-Related Diseases

The present invention pertains to methods of using the compounds and pharmaceutical compositions thereof in the treatment and prevention of amyloid-related diseases. The pharmaceutical compositions of the invention may be administered therapeutically or prophylactically to treat diseases associated with amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, or AH amyloid protein) fibril formation, aggregation or deposition.

The pharmaceutical compositions of the invention may act to ameliorate the course of an amyloid-related disease using any of the following mechanisms (this list is meant to be illustrative and not limiting): slowing the rate of amyloid fibril formation or deposition; lessening the degree of amyloid deposition; inhibiting, reducing, or preventing amyloid fibril formation; inhibiting neurodegeneration or cellular toxicity induced by amyloid; inhibiting amyloid induced inflammation; enhancing the clearance of amyloid from the brain; enhancing degradation of Aβ in the brain; or favoring clearance of amyloid protein prior to its organization in fibrils.

“Modulation” of amyloid deposition includes both inhibition, as defined above, and enhancement of amyloid deposition or fibril formation. The term “modulating” is intended, therefore, to encompass prevention or stopping of amyloid formation or accumulation, inhibition or slowing down of further amyloid formation or accumulation in a subject with ongoing amyloidosis, e.g., already having amyloid deposition, and reducing or reversing of amyloid formation or accumulation in a subject with ongoing amyloidosis; and enhancing amyloid deposition, e.g., increasing the rate or amount of amyloid deposition in vivo or in vitro. Amyloid-enhancing compounds may be useful in animal models of amyloidosis, for example, to make possible the development of amyloid deposits in animals in a shorter period of time or to increase amyloid deposits over a selected period of time. Amyloid-enhancing compounds may be useful in screening assays for compounds which inhibit amyloidosis in vivo, for example, in animal models, cellular assays and in vitro assays for amyloidosis. Such compounds may be used, for example, to provide faster or more sensitive assays for compounds. Modulation of amyloid deposition is determined relative to an untreated subject or relative to the treated subject prior to treatment.

“Inhibition” of amyloid deposition includes preventing or stopping of amyloid formation, e.g., fibrillogenesis, clearance of amyloid, e.g., soluble Aβ from brain, inhibiting or slowing down of further amyloid deposition in a subject with amyloidosis, e.g., already having amyloid deposits, and reducing or reversing amyloid fibrillogenesis or deposits in a subject with ongoing amyloidosis. Inhibition of amyloid deposition is determined relative to an untreated subject, or relative to the treated subject prior to treatment, or, e.g., determined by clinically measurable improvement, e.g., or in the case of a subject with brain amyloidosis, e.g., an Alzheimer's or cerebral amyloid angiopathy subject, stabilization of cognitive function or prevention of a further decrease in cognitive function (i.e., preventing, slowing, or stopping disease progression), or improvement of parameters such as the concentration of Aβ or tau in the CSF.

As used herein, “treatment” of a subject includes the application or administration of a composition of the invention to a subject, or application or administration of a composition of the invention to a cell or tissue from a subject, who has an amyloid-related disease or condition, has a symptom of such a disease or condition, or is at risk of (or susceptible to) such a disease or condition, with the purpose of curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of dementia. The treatment or amelioration of symptoms can be based on objective or subjective parameters; including the results of a physical examination, a psychiatric evaluation, or a cognition test such as CDR, MMSE, ADAS-Cog, DAD, or another test known in the art. For example, the methods of the invention successfully treat a subject's dementia by slowing the rate of or lessening the extent of cognitive decline.

In one embodiment, the term “treating” includes maintaining a subject's CDR rating at its base line rating or at 0. In another embodiment, the term treating includes decreasing a subject's CDR rating by about 0.25 or more, about 0.5 or more, about 1.0 or more, about 1.5 or more, about 2.0 or more, about 2.5 or more, or about 3.0 or more. In another embodiment, the term “treating” also includes reducing the rate of the increase of a subject's CDR rating as compared to historical controls. In another embodiment, the term includes reducing the rate of increase of a subject's CDR rating by about 5% or more, about 10% or more, about 20% or more, about 25% or more, about 30% or more, about 40% or more, about 50% or more, about 60% or more, about 70% or more, about 80% or more, about 90% or more, or about 100%, of the increase of the historical or untreated controls.

In another embodiment, the term “treating” also includes maintaining a subject's score on the MMSE. The term “treating” includes increasing a subject's MMSE score by about 1, about 2, about 3, about 4, about 5, about 7.5, about 10, about 12.5, about 15, about 17.5, about 20, or about 25 points. The term also includes reducing the rate of the decrease of a subject's MMSE score as compared to historical controls. In another embodiment, the term includes reducing the rate of decrease of a subject's MMSE score by about 5% or less, about 10% or less, about 20% or less, about 25% or less, about 30% or less, about 40% or less, about 50% or less, about 60% or less, about 70% or less, about 80% or less, about 90% or less or about 100% or less, of the decrease of the historical or untreated controls.

In another embodiment, the term “treating” also includes maintaining a subject's score on the DAD. The term “treating” includes increasing a subject's DAD score by about 1, about 5, about 10, about 15, about 20, about 30, about 35, about 40, about 50, about 60, about 70, or about 80 points. The term also includes reducing the rate of the decrease of a subject's DAD score as compared to historical controls. In another embodiment, the term includes reducing the rate of decrease of a subject's DAD score by about 5% or less, about 10% or less, about 20% or less, about 25% or less, about 30% or less, about 40% or less, about 50% or less, about 60% or less, about 70% or less, about 80% or less, about 90% or less or about 100% or less, of the decrease of the historical or untreated controls.

In yet another embodiment, the term “treating” includes maintaining a subject's score on the ADAS-Cog. The term “treating” includes decreasing a subject's ADAS-Cog score by about 1 point or greater, by about 2 points or greater, by about 3 points or greater, by about 4 points or greater, by about 5 points or greater, by about 7.5 points or greater, by about 10 points or greater, by about 12.5 points or greater, by about 15 points or greater, by about 17.5 points or greater, by about 20 points or greater, or by about 25 points or greater. The term also includes reducing the rate of the increase of a subject's ADAS-Cog score as compared to historical controls. In another embodiment, the term includes reducing the rate of increase of a subject's ADAS-Cog score by about 5% or more, about 10% or more, about 20% or more, about 25% or more, about 30% or more, about 40% or more, about 50% or more, about 60% or more, about 70% or more, about 80% or more, about 90% or more or about 100% of the increase of the historical or untreated controls.

In another embodiment, the term “treating” e.g., for AA or AL amyloidosis, includes an increase in serum creatinine, e.g., an increase of creatinine clearance of 10% or greater, 20% or greater, 50% or greater, 80% or greater, 90% or greater, 100% or greater, 150% or greater, 200% or greater. The term “treating” also may include remission of nephrotic syndrome (NS). It may also include remission of chronic diarrhea and/or a gain in body weight, e.g., by 10% or greater, 15% or greater, or 20% or greater.

Without wishing to be bound by theory, in some aspects the pharmaceutical compositions of the invention contain a compound that prevents or inhibits amyloid fibril formation, either in the brain or other organ of interest (acting locally) or throughout the entire body (acting systemically). Pharmaceutical compositions of the invention may be effective in controlling amyloid deposition either following their entry into the brain (following penetration of the blood brain barrier) or from the periphery. When acting from the periphery, a compound of a pharmaceutical composition may alter the equilibrium of amyloidogenic peptide between the brain and the plasma so as to favor the exit of amyloidogenic peptide from the brain. It may also favor clearance (or catabolism) of the amyloid protein (soluble), and then prevent amyloid fibril formation and deposition due to a reduction of the amyloid protein pool in a specific organ, e.g., liver, spleen, pancreas, kidney, joints, brain, etc. An increase in the exit of amyloidogenic peptide from the brain would result in a decrease in amyloidogenic peptide brain concentration and therefore favor a decrease in amyloidogenic peptide deposition. In particular, an agent may lower the levels of amyloid β peptides, e.g., both Aβ40 and Aβ42 in the CSF and the plasma, or the agent may lower the levels of amyloid β peptides, e.g., Aβ40 and Aβ42 in the CSF and increase it in the plasma. Alternatively, compounds that penetrate the brain could control deposition by acting directly on brain amyloidogenic peptide e.g., by maintaining it in a non-fibrillar form or favoring its clearance from the brain, by increasing its degradation in the brain, or protecting brain cells from the detrimental effect of amyloidogenic peptide. An agent can also cause a decrease of the concentration of the amyloid protein (i.e., in a specific organ so that the critical concentration needed to trigger amyloid fibril formation or deposition is not reached). Furthermore, the compounds described herein may inhibit or reduce an interaction between amyloid and a cell surface constituent, for example, a glycosaminoglycan or proteoglycan constituent of a basement membrane, whereby inhibiting or reducing this interaction produces the observed neuroprotective and cell-protective effects. For example, the compound may also prevent an amyloid peptide from binding or adhering to a cell surface, a process which is known to cause cell damage or toxicity. Similarly, the compound may block amyloid-induced cellular toxicity or microglial activation or amyloid-induced neurotoxicity, or inhibit amyloid induced inflammation. The compound may also reduce the rate or amount of amyloid aggregation, fibril formation, or deposition, or the compound lessens the degree of amyloid deposition. The compound may also be capable of blocking formation of oligomers and inhibit oligomer induced toxicity. The foregoing mechanisms of action should not be construed as limiting the scope of the invention inasmuch as the invention may be practiced without such information.

The Aβ peptide has been shown by several groups to be highly toxic to neurons. Amyloid plaques are directly associated with reactive gliosis, dystrophic neurites and apoptotic cells, suggesting that plaques induce neurodegenerative changes. Neurotoxicity may eventually disrupt or even kill neurons. In vitro, Aβ has been shown to induce apoptosis in many different neuronal cell types, such as rat PC-12 cells, primary rat hippocampal and cortical cultures, and the predifferentiated human neurotype SH-SY5Y cell line (Dickson D W (2004) J Clin Invest 114:23-7; Canu et al. (2003) Cerebellum 2:270-278; Li et al. (1996) Brain Research 738:196-204). Numerous reports have shown that Aβ fibrils can induce neurodegeneration, and it has been shown that neuronal cells exposed in vitro to Aβ can become apoptotic (Morgan et al. (2004) Prog. Neurobiol. 74:323-349; Stefani et al. (2003) J. Mol Med. 81:678-99; La Ferla et al. (1997) J. Clin. Invest. 100(2):310-320). In Alzheimer's disease, a progressive neuronal cell loss accompanies the deposition of Aβ amyloid fibrils in senile plaques.

In yet another aspect, the invention pertains to a method for inhibiting Aβ-induced neuronal cell death by administering an effective amount of a compound of the present invention.

Another aspect of the invention pertains to a method for providing neuroprotection to a subject having an Aβ-amyloid related disease, e.g. Alzheimer's disease, that includes administering an effective amount of a compound of the present invention to the subject, such that neuroprotection is provided.

In another aspect, methods for inhibiting Aβ-induced neuronal cell death are provided that include administration of an effective amount of a compound of the present invention to a subject such that neuronal cell death is inhibited.

In another aspect, methods for treating a disease state characterized by Aβ-induced neuronal cell death in a subject are provided, e.g., by administering an effective amount of a compound of the present invention. Non-limiting examples of such disease states include Alzheimer's disease and Aβ-amyloid related diseases.

The term “neuroprotection” includes protection of neuronal cells of a subject from Aβ-induced cell death, e.g., cell death induced directly or indirectly by an Aβ peptide. Aβ-induced cell death may result in initiation of processes such as, for example: the destabilization of the cytoskeleton; DNA fragmentation; the activation of hydrolytic enzymes, such as phospholipase A2; activation of caspases, calcium-activated proteases and/or calcium-activated endonucleases; inflammation mediated by macrophages; calcium influx into a cell; membrane potential changes in a cell; the disruption of cell junctions leading to decreased or absent cell-cell communication; and the activation of expression of genes involved in cell death, e.g., immediate-early genes.

The term “amyloid-β disease” (or “amyloid-β related disease,” which terms as used herein are synonymous) may be used for mild cognitive impairment; vascular dementia; early Alzheimer's disease; Alzheimer's disease, including sporadic (non-hereditary) Alzheimer's disease and familial (hereditary) Alzheimer's disease; age-related cognitive decline; cerebral amyloid angiopathy (“CAA”); hereditary cerebral hemorrhage; senile dementia; Down's syndrome; inclusion body myositis (“IBM”); or age-related macular degeneration (“ARMD”).

Cerebral amyloid angiopathy (“CAA”) refers to the specific deposition of amyloid fibrils in the walls of leptomingeal and cortical arteries, arterioles and in capillaries and veins. It is commonly associated with Alzheimer's disease, Down's syndrome and normal aging, as well as with a variety of familial conditions related to stroke or dementia (see Frangione, et al., Amyloid: J. Protein Folding Disord. 8, Suppl. 1, 36-42 (2001)). CAA can occur sporadically or be hereditary. Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia. Cerebral amyloid angiopathy is known to be associated with cerebral hemorrhage (or hemorrhagic stroke).

Also, the invention relates to a method for preventing or inhibiting amyloid deposition in a subject. For example, such a method comprises administering to a subject a therapeutically effective amount of a compound capable of reducing the concentration of amyloid (e.g., AL amyloid protein (k or K-chain related, e.g., amyloid λ, amyloid κ, amyloid κIV, amyloid λVI, amyloid γ, amyloid γ1), Aβ, IAPP, β₂M, AA, AH amyloid protein, or other amyloids), such that amyloid accumulation or deposition is prevented or inhibited.

In another aspect, the invention relates to a method for preventing, reducing, or inhibiting amyloid deposition in a subject. For example, such a method comprises administering to a subject a therapeutically effective amount of a compound capable of inhibiting amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, AH amyloid protein, or other amyloids), such that amyloid deposition is prevented, reduced, or inhibited.

The invention also relates to a method for modulating, e.g., minimizing, amyloid-associated damage to cells, comprising the step of administering a compound capable of reducing the concentration of amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, AH amyloid protein, or another amyloid), such that said amyloid-associated damage to cells is modulated. In certain aspects of the invention, the methods for modulating amyloid-associated damage to cells comprise a step of administering a compound capable of reducing the concentration of amyloid or reducing the interaction of an amyloid with a cell surface.

The invention also includes a method for directly or indirectly preventing cell death in a subject, the method comprising administering to a subject a therapeutically effective amount of a compound capable of preventing amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, AH amyloid protein, or other amyloid) mediated events that lead, directly or indirectly, to cell death.

In an embodiment, the method is used to treat Alzheimer's disease (e.g. sporadic or familial AD). The method can also be used prophylactically or therapeutically to treat other clinical occurrences of amyloid-β deposition, such as in Down's syndrome individuals and in patients with cerebral amyloid angiopathy (“CAA”) or hereditary cerebral hemorrhage.

The compounds of the invention may be used prophylactically or therapeutically in the treatment of disorders in which amyloid-beta peptide is abnormally deposited at non-neurological locations, such as treatment of IBM by delivery of the compounds to muscle fibers, or treatment of macular degeneration by delivery of the compound(s) of the invention to the basal surface of the retinal pigmented epithelium.

The present invention also provides a method for modulating amyloid-associated damage to cells, comprising the step of administering a compound capable of reducing the concentration of Aβ, or capable of minimizing the interaction of Aβ (soluble oligomeric or fibrillary) with the cell surface, such that said amyloid-associated damage to cells is modulated. In certain aspects of the invention, the methods for modulating amyloid-associated damage to cells comprise a step of administering a compound capable of reducing the concentration of Aβ or reducing the interaction of Aβ with a cell surface.

In accordance with the present invention, there is further provided a method for preventing cell death in a subject, said method comprising administering to a subject a therapeutically effective amount of a compound capable of preventing Aβmediated events that lead, directly or indirectly, to cell death.

The present invention also provides a method for modulating amyloid-associated damage to cells, comprising the step of administering a compound capable of reducing the concentration of IAPP, or capable of minimizing the interaction of IAPP (soluble oligomeric or fibrillary) with the cell surface, such that said amyloid-associated damage to cells is modulated. In certain aspects of the invention, the methods for modulating amyloid-associated damage to cells comprise a step of administering a compound capable of reducing the concentration of IAPP or reducing the interaction of IAPP with a cell surface.

In accordance with the present invention, there is further provided a method for preventing cell death in a subject, said method comprising administering to a subject a therapeutically effective amount of a compound capable of preventing IAPP (monomeric, oligomeric, or fibrillar)-mediated events that lead, directly or indirectly, to cell death.

This invention also provides methods and compositions which are useful in the treatment of amyloidosis. The methods of the invention involve administering to a subject a therapeutic compound which inhibits amyloid deposition. Accordingly, the compositions and methods of the invention are useful for inhibiting amyloidosis in disorders in which amyloid deposition occurs. The methods of the invention can be used therapeutically to treat amyloidosis or can be used prophylactically in a subject susceptible to (hereditary) amyloidosis or identified as being at risk to develop amyloidosis, e.g., hereditary, or identified as being at risk to develop amyloidosis. In certain embodiments, the invention includes a method of inhibiting an interaction between an amyloidogenic protein and a constituent of basement membrane to inhibit amyloid deposition. The constituent of basement membrane is a glycoprotein or proteoglycan, preferably heparan sulfate proteoglycan. A therapeutic compound used in this method may interfere with binding of a basement membrane constituent to a target binding site on an amyloidogenic protein, thereby inhibiting amyloid deposition.

In some aspects, the methods of the invention involve administering to a subject a therapeutic compound which inhibits amyloid deposition. “Inhibition of amyloid deposition,” includes the prevention of amyloid formation, inhibition of further amyloid deposition in a subject with ongoing amyloidosis and reduction of amyloid deposits in a subject with ongoing amyloidosis. Inhibition of amyloid deposition is determined relative to an untreated subject or relative to the treated subject prior to treatment. In an embodiment, amyloid deposition is inhibited by inhibiting an interaction between an amyloidogenic protein and a constituent of basement membrane. “Basement membrane” refers to an extracellular matrix comprising glycoproteins and proteoglycans, including laminin, collagen type IV, fibronectin, perlecan, agrin, dermatan sulfate, and heparan sulfate proteoglycan (HSPG). In one embodiment, amyloid deposition is inhibited by interfering with an interaction between an amyloidogenic protein and a sulfated glycosaminoglycan such as HSPG, dermatan sulfate, perlecan or agrin sulfate. Sulfated glycosaminoglycans are known to be present in all types of amyloids (see Snow, et al. Lab. Invest. 56, 120-23 (1987)) and amyloid deposition and HSPG deposition occur coincidentally in animal models of amyloidosis (see Snow, et al. Lab. Invest. 56, 665-75 (1987) and Gervais, F. et al. Curr. Med. Chem., 3, 361-370 (2003)). Consensus binding site motifs for HSPG in amyloidogenic proteins have been described (see, e.g., Cardin and Weintraub Arteriosclerosis 9, 21-32 (1989)).

The ability of a compound to prevent or block the formation or deposition of amyloid may reside in its ability to bind to non-fibrillar, soluble amyloid protein and to maintain its solubility.

The ability of a therapeutic compound of the invention to inhibit an interaction between an amyloidogenic protein and a glycoprotein or proteoglycan constituent of a basement membrane can be assessed by an in vitro binding assay, such as that described in U.S. Pat. No. 5,164,295, the contents of which are hereby incorporated by reference. Alternatively, the ability of a compound to bind to an amyloidogenic protein or to inhibit the binding of a basement membrane constituent (e.g. HSPG) to an amyloidogenic protein (e.g. Aβ) can be measured using a mass spectrometry assay where soluble protein, e.g. Aβ, IAPP, β₂M is incubated with the compound. A compound which binds to, e.g. Aβ, will induce a change in the mass spectrum of the protein. Exemplary protocols for a mass spectrometry assay employing Aβ and IAPP can be found in the Examples, the results of which are provided in Table 3. The protocol can readily be modified to adjust the sensitivity of the data, e.g., by adjusting the amount of protein and/or compound used. Thus, e.g., binding might be detected for test compounds noted as not having detectable binding employing less sensitive test protocols.

Alternative methods for screening compounds exist and can readily be used by a skilled practitioner to provide an indication of the ability of test compounds to bind to, e.g., fibrillar Aβ. One such screening assay is an ultraviolet absorption assay. In an exemplary protocol, a test compound (20 μM) is incubated with 50 μM Aβ(1-40) fibers for 1 hour at 37° C. in Tris buffered saline (20 mM Tris, 150 mM NaCl, pH 7.4 containing 0.01 sodium azide). Following incubation, the solution is centrifuged for 20 minutes at 21,000 g to sediment the Aβ(1-40) fibers along with any bound test compound. The amount of test compound remaining in the supernatant can then be determined by reading the absorbance. The fraction of test compound bound can then be calculated by comparing the amount remaining in the supernatants of incubations with Aβ to the amount remaining in control incubations which do not contain Aβ fibers. Thioflavin T and Congo Red, both of which are known to bind to Aβ fibers, may be included in each assay run as positive controls. Before assaying, test compounds can be diluted to 40 μM, which would be twice the concentration in the final test, and then scanned using the Hewlett Packard 8453 UV/VIS spectrophotometer to determine if the absorbance is sufficient for detection.

In another embodiment, the invention pertains to a method for improving cognition in a subject suffering from an amyloid-related disease. The method includes administering an effective amount of a therapeutic compound of the invention, such that the subject's cognition is improved. The subject's cognition can be tested using methods known in the art such as the Clinical Dementia Rating (“CDR”), Mini-Mental State Examination (“MMSE”), Disability Assessment for Dementia (“DAD”) and the Alzheimer's Disease Assessment Scale-Cognition (“ADAS-Cog”).

In another embodiment, the invention pertains to a method for treating a subject for an amyloid-related disease. The method includes administering a cognitive test to a subject prior to administration of a compound of the invention, administering an effective amount of a compound of the invention to the subject, and administering a cognitive test to the subject subsequent to administration of the compound, such that the subject is treated for the amyloid-related disease, wherein the subject's score on said cognitive test is improved.

“Improvement,” “improved” or “improving” in cognition is present within the context of the present invention if there is a statistically significant difference in the direction of normality between the performance of subjects treated using the methods of the invention as compared to members of a placebo group, historical control, or between subsequent tests given to the same subject.

In one embodiment, a subject's CDR is maintained at 0. In another embodiment, a subject's CDR is decreased (e.g., improved) by about 0.25 or more, about 0.5 or more, about 1.0 or more, about 1.5 or more, about 2.0 or more, about 2.5 or more, or about 3.0 or more. In another embodiment, the rate of increase of a subject's CDR rating is reduced by about 5% or more, about 10% or more, about 20% or more, about 25% or more, about 30% or more, about 40% or more, about 50% or more, about 60% or more, about 70% or more, about 80% or more, about 90% or more, or about 100% or more of the increase of the historical or untreated controls.

In one embodiment, a subject's score on the MMSE is maintained. Alternatively, the subject's score on the MMSE may be increased by about 1, about 2, about 3, about 4, about 5, about 7.5, about 10, about 12.5, about 15, about 17.5, about 20, or about 25 points. In another alternative, the rate of the decrease of a subject's MMSE score as compared to historical controls is reduced. For example, the rate of the decrease of a subject's MMSE score may be reduced by about 5% or more, about 10% or more, about 20% or more, about 25% or more, about 30% or more, about 40% or more, about 50% or more, about 60% or more, about 70% or more, about 80% or more, about 90% or more, or about 100% or more of the decrease of the historical or untreated controls.

In one embodiment, a subject's score on the DAD is maintained. Alternatively, the subject's score on the DAD may be increased by about 1, about 2, about 3, about 4, about 5, about 7.5, about 10, about 15, about 20, about 30, about 40, or about 50 or more points. In another alternative, the rate of the decrease of a subject's DAD score as compared to historical controls is reduced. For example, the rate of the decrease of a subject's DAD score may be reduced by about 5% or more, about 10% or more, about 20% or more, about 25% or more, about 30% or more, about 40% or more, about 50% or more, about 60% or more, about 70% or more, about 80% or more, about 90% or more, or about 100% or more of the decrease of the historical or untreated controls.

In one embodiment, the invention pertains to a method for treating, slowing or stopping an amyloid-related disease associated with cognitive impairment, by administering to a subject an effective amount of a therapeutic compound of the invention, wherein the annual deterioration of the subject's cognition as measured by ADAS-Cog is less than 8 points per year, less the 6 points per year, less than 5 points per year, less than 4 points per year, or less than 3 points per year. In a further embodiment, the invention pertains to a method for treating, slowing or stopping an amyloid-related disease associated with cognition by administering an effective amount of a therapeutic compound of the invention such that the subject's cognition as measured by ADAS-Cog remains constant over a year. “Constant” includes fluctuations of no more than 2 points. Remaining constant includes fluctuations of two points or less in either direction. In a further embodiment, the subject's cognition improves by 2 points or greater per year, 3 points or greater per year, 4 point or greater per year, 5 points or greater per year, 6 points or greater per year, 7 points or greater per year, 8 points or greater per year, etc. as measured by the ADAS-Cog. In another alternative, the rate of the increase of a subject's ADAS-Cog score as compared to historical controls is reduced. For example, the rate of the increase of a subject's ADAS-Cog score may be reduced by about 5% or more, about 10% or more, about 20% or more, about 25% or more, about 30% or more, about 40% or more, about 50% or more, about 60% or more, about 70% or more, about 80% or more, about 90% or more or about 100% of the increase of the historical or untreated controls.

In another embodiment, the ratio of Aβ42:Aβ40 in the CSF or plasma of a subject decreases by about 15% or more, about 20% or more, about 25% or more, about 30% or more, about 35% or more, about 40% or more, about 45% or more, or about 50% or more. In another embodiment, the levels of Aβ in the subject's cerebrospinal fluid decrease by about 15% or more, about 25% or more, about 35% or more, about 45% or more, about 55% or more, about 75% or more, or about 90% or more.

In one embodiment, the compounds of the invention selectively bind to fibrillar amyloid. The methods of the invention can be used to detect amyloid deposits and other occurrences of fibrillar amyloid.

In another embodiment, the compounds of the invention selectively bind to soluble amyloid. Compounds of the invention which bind to soluble amyloid can be used to observe the amyloid as it travels through the subject, forms fibrils, and is deposited. The compounds can also be used to test for the presence of soluble amyloid and/or fibrillar amyloid ex vivo.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values in these values and ranges may also be the upper or lower limits of a range.

Furthermore, the invention pertains to any novel chemical compound described herein. That is, the invention relates to novel compounds, and novel methods of their use as described herein, which are within the scope of the Formulae disclosed herein, and which are not disclosed in the cited Patents and Patent Applications.

Use of Compounds of the Invention in Imaging Methods

The binding properties of amino alkyl sulfonate moieties may be combined with imaging properties of fluorine moieties to provide compounds that are not only useful for the treatment of diseases (e.g., amyloid-related diseases), but that can also be used as an NMR detectable agent for a number of diagnostic and therapeutic uses (e.g., detection of amyloid, diagnosis of disease and/or diagnosis of disease state).

Accordingly, the invention provides a detectable agent (e.g., a contrast agent, imaging probe or diagnostic reagent) that binds or otherwise associates with a moiety of interest (e.g., Aβ, IAPP and β2M) in a subject or sample or tissue or cell, thus allowing detection of the compound and the moiety of interest. Use of such compounds can provide information such as the presence, location, density and/or amount of a moiety of interest (e.g., an amyloid). Such information can allow diagnosis of a disease or disease state or detect a predisposition to such a disease or disease state. Accordingly, the present invention provides methods of using the compounds of the invention to detect, diagnose, and monitor disease or a predisposition to a disease or disease state. These methods can be used with any of the subject populations described herein, to detect any of the amyloid proteins described and/or to treat any of the amyloid related diseases described herein. These methods may include employing any of the compounds described herein.

The compounds of the invention may be used as contrast agents, imaging probes and/or diagnostic reagents. For example, the compounds of the invention may be used in accordance with the method of the present invention to detect or locate amyloid and/or amyloid deposits. The compounds of the invention can be used to enhance imaging, e.g., of amyloid fibril formation and/or the surrounding environment of amyloid.

The term “imaging probe” refers to a probe that can be used in conjunction with an imaging technique. Exemplary probes may include the compounds of the invention comprising a ¹⁹F isotope (and/or another isotope which has properties which allow it to be detected by imaging techniques), which can be used in conjunction with imaging techniques such as Magnetic Resonance Imaging (MRI), Magnetic Resonance Spectroscopy (MRS), Positron Emission Tomography (PET), or ultrasound (US). Imaging probes can be used to image or probe biological or other structures.

The term “diagnostic reagent” refers to agents that can be used to diagnose or aid in the diagnosis of a disease or disorder (e.g., an amyloid-related disease or disorder). By way of example, a diagnostic reagent can be used to provide information regarding the stage, progression or regression of the disease or disorder and/or to identify particular locations of or localizations of disease or disorder related moieties (e.g., locations of or localizations of amyloid proteins).

The term “contrast agent” refers to agents that can enhance imaging of cells, organs, and other structures. In fluoroscopy, contrast agents are used to enhance the imaging of otherwise radiolucent tissues. Generally, fluoroscopic contrast agents work by x-ray absorption. For NMR or MRI image enhancement, contrast agents generally shorten either the T₁ or T₂ proton relaxation times, giving rise to intensity enhancement in appropriately weighted images.

The fluorinated compounds of the invention can include one, a plurality, or even a maximum number of chemically equivalent fluorines on one or more substituents resonating at one or only a few frequencies, e.g., from trifluoromethyl functions. Spectral aspects of fluorinated compounds generally are known and described in the literature. See e.g., Sotak, C. H. et al., MAGN. RESON. MED. 29:188-195 (1993).

In one embodiment, the compounds of the invention are water soluble. This can enhance the functionality of the compounds of the invention in many biomedical settings, as it can, e.g., obviate the need for emulsifiers. Amino alkyl sulfonic acids generally have water solubilities that are relatively independent of pH: A sulfonic acid group typically has a pKa of about 2. Accordingly, the compounds of the present invention generally are water soluble, biocompatible, and/or able to cross the blood brain barrier by active or passive transport.

Methods of Imaging

Nuclear magnetic resonance (NMR) techniques are finding increasing use in medical diagnostics. NMR imaging, or magnetic resonance imaging (MRI) as it is sometimes known, has been found to be useful in the detection of a variety of diseases and disorders. MRI has several advantages over other imaging techniques. For example, unlike computerized tomographic methods, MRI does not employ ionizing radiation, and therefore is believed to be safer. Also, MRI can provide more information about soft tissue than can some other imaging methods.

Nuclear magnetic resonance (NMR) techniques permit the assessment of biochemical, functional, and physiological information from patients. Magnetic resonance imaging (MRI) of tissue water, e.g., can be used to measure perfusion and diffusion with submillimeter resolution. Magnetic resonance spectroscopy may be applied to the assessment of tissue metabolites that contain protons, phosphorus, fluorine, or other nuclei. The combination of imaging and spectroscopy technologies has lead to spectroscopic imaging techniques that are capable of mapping proton metabolites at resolutions as small as 0.25 cm³ (Zakian K L et al. Semin Radiat Oncol.; 11(1):3-15, 2001).

The majority of the NMR techniques developed so far have been based on imaging of hydrogen nuclei. However, other nuclei offer potential advantages with respect to NMR. Fluorine in particular is of interest. The fluorine nucleus offers a strong NMR signal magnitude (high gyromagnetic ratio) second only to that of protons. Virtually no imageable fluorine exists naturally in the human body, so no background signal exists; any detectable signal comes only from whatever fluorine has been administered to the subject.

Fluorine-19 (¹⁹F) is a stable isotope and is naturally abundant, such that isotopic enrichment is generally unnecessary. Because its gyromagnetic ratio is about 94% that of hydrogen, existing equipment designed to image protons can be inexpensively adapted for ¹⁹F.

Apolar oxygen imparts paramagnetic relaxation effects on ¹⁹F nuclei associated with spin-lattice relaxation rates (R₁) and chemical shifts. This effect is proportional to the partial pressure of O₂ (pO₂). ¹⁹F NMR can therefore probe the oxygen environment of specific fluorinated compounds of the invention in cells and other biological structures. The term “MRI,” as used herein, also includes functional MRI (fMRI) which is an imaging technique used to study one or more functions of interest over time to gain information about the functioning of the area of interest. Accordingly, the methods of the invention include administration of a plurality of MRIs over time. The method can include analyzing the effect of any number of compounds and therapies on a subject. The method can thus be used, e.g., to study the effectiveness of a compound of the invention, or other therapeutic compounds, in inhibiting amyloid deposition, by employing fMRI to assess whether such compounds are effective at modulating amyloid deposition over time.

Methods of MRI imaging that can be used in connection with the present invention are described, e.g., in The Contrast Media Manual, (1992, R. W. Katzberg, Williams and Wilkins, Baltimore, Md.), especially Chapter 13 (“Magnetic Resonance Contrast Agents”).

In one embodiment of the present invention, an effective amount of a formulation or composition comprising a compound of the invention in a pharmaceutically acceptable carrier is administered to a patient, and the patient is scanned. The term “amount effective to provide a detectable NMR signal”, refers to a non-toxic amount of compound sufficient to allow detection or to enhance or alter a MRI image. The compound can be administered in an amount that permits detection of the compounds or structures of interest (e.g., amyloid protein or amyloid plaques) and/or enhance detection or visualization of these compounds or structures as well as the surrounding organs or tissues. In one embodiment, the patient is a mammal, e.g., a human or non-human mammal. In another embodiment, an effective amount of compound is administered or introduced to a tissue, or one or more cells, or a sample, e.g., that include a moiety of interest such as amyloid proteins.

The compounds of the invention may also be radiopharmaceutical compounds. Radiopharmaceuticals are drugs containing a radionuclide (e.g., ¹⁸F), and are used in the field of radiology known as nuclear medicine for the diagnosis or therapy of various diseases. In vivo diagnostic information may be obtained by administration, e.g., by intravenous injection, of a radiopharmaceutical and determining its biodistribution using a radiation-detecting camera. In PET, radio nuclides, typically fluorine-18, are incorporated into substances so as to produce radiopharmaceuticals which are ingested by the patient. As the radio nuclides decay, positrons are emitted and they collide, in a very short distance, with an electron and become annihilated and converted into two photons, or gamma rays, traveling linearly in opposite directions to one another with each ray having an energy of 511 KeV. PET scanners typically include laterally spaced rings with detectors which encircle the patient. A typical detector within the ring is a BgO crystal in front of a photomultiplier tube. Each ring is thus able to discern an annihilation event occurring in a single plane. The analog PMT signals are analyzed by coincidence detection circuits to detect coincident or simultaneous signals generated by PMT's on opposite sides of the patient, i.e., opposed detectors on the ring. Specifically, when two opposed detectors detect simultaneous 511 KeV events, a line passing through both detectors establishes a line of response (LOR). By processing a number of LORs indicative of annihilation events an image is reconstructed of the organ using computed tomographic techniques.

There are numerous PET scanner coincident detector schemes such as illustrated in U.S. Pat. Nos. 4,395,635; 4,864,140; 5,241,181; and 5,532,489 which determine if two photons struck a detector within a very short time of one another to establish a positron annihilation event.

In PET, radio nuclides, typically fluorine-18 are incorporated into the compounds of the invention which may be ingested by or injected into the patient. As the radio nuclides decay, positrons are emitted and they collide, in a very short distance, with an electron and become annihilated and converted into two photons, or gamma rays, traveling linearly in opposite directions to one another with each ray having an energy of 511 KeV. PET scanners typically comprise, laterally spaced rings which encircle the patient. Each ring contains detectors extending thereabout. A typical detector within the ring is a BgO crystal in front of a photomultiplier tube. Each ring is thus able to discern an annihilation event occurring in a single plane. The analog PMT signals are analyzed by coincidence detection circuits to detect coincident or simultaneous signals generated by PMT's on opposite sides of the patient, i.e., opposed detectors on the ring. Specifically, when two opposed detectors detect simultaneous 511 KeV events, a line passing through both detectors establishes a line of response (LOR). By processing a number of LORs indicative of annihilation events an image is reconstructed of the organ using computed tomographic techniques. There are numerous PET scanner and detector schemes illustrated in U.S. Pat. Nos. 4,395,635; 4,864,140; 5,241,181; and 5,532,489. In SPECT, two different radio pharmaceuticals having photons of different energy levels (e.g., technetium and thallium) may be used simultaneously. See also U.S. Pat. Nos. 5,532,489, 5,272,343, 5,241,181, 5,512, 755, 5,345,082, 5,023,895, 4,864,140, 5,323,006, 4,675,526, and 4,395,635.

PET imaging can also be used to monitor stress non-invasively (Eckelman, W. et al. Annals of the New York Academy of Sciences (2004), 1018(Stress), 487-494; Schreckenberger, Eur. J. Nuc. Med. Mol. Imag. (2004), 31(8), 1128-1135; Mirzaei, S.; et al. Curr. Alzheimer Res. (2004), 1(3), 219-229; Mathis, C. A et al. Curr. Pharm. Des. (2004), 10(13), 1469-1492).

Ultrasound is another valuable diagnostic imaging technique and provides certain advantages over other diagnostic techniques. Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that may detect sound waves having a frequency of one megahertz (mHz) to ten mHz. The detected waves may be integrated into an image which is quantitated and the quantitated waves converted into an image of the tissue being studied. Ultrasound also generally involves the use of contrast agents such as suspensions of solid particles, emulsified liquid droplets, and gas-filled bubbles or vesicles.

Ultrasound imaging modalities which may be used in accordance with the invention include two- and three-dimensional imaging techniques such as B-mode imaging (for example, using the time-varying amplitude of the signal envelope generated from the fundamental frequency of the emitted ultrasound pulse, from sub-harmonics or higher harmonics thereof or from sum or difference frequencies derived from the emitted pulse and such harmonics, images generated from the fundamental frequency or the second harmonic thereof being preferred), color Doppler imaging and Doppler amplitude imaging, and combinations of the two latter with any of the modalities (techniques) above. To reduce the effects of movement, successive images of tissues such as the heart or kidney may be collected with the aid of suitable synchronization techniques (e.g., gating to the ECG or respiratory movement of the subject). Measurement of changes in resonance frequency or frequency absorption which accompany arrested or retarded microbubbles may also usefully be made to detect the contrast agent.

In the case of diagnostic applications, such as ultrasound, energy, such as ultrasonic energy, is applied to at least a portion of the patient to image the target tissue. A visible image of an internal region of the patient is then obtained, such that the presence or absence of diseased tissue may be ascertained.

In addition to the pulsed method, continuous wave ultrasound such as Power Doppler may be applied. This may be particularly useful where rigid vesicles, for example, vesicles formulated from polymethyl methacrylate, are used. In this case, the relatively higher energy of the Power Doppler may be made to resonate the vesicles and thereby promote their rupture. This may create acoustic emissions which may be in the subharmonic or ultraharmonic range or, in some cases, in the same frequency as the applied ultrasound. In addition, the process of vesicle rupture may be used to transfer kinetic energy to the surface, for example of a plaque, to promote amyloid plaque lysis which may be useful in the treatment of certain amyloid related diseases. Thus, therapeutic plaque lysis may be achieved during a combination of diagnostic and therapeutic ultrasound. Spectral Doppler may also be used. The levels of energy from diagnostic ultrasound may be insufficient to promote the rupture of vesicles and to facilitate release and cellular uptake of the bioactive agents. As noted above, diagnostic ultrasound may involve the application of one or more pulses of sound. Pauses between pulses permit the reflected sonic signals to be received and analyzed. The limited number of pulses used in diagnostic ultrasound limits the effective energy which is delivered to the tissue that is being studied.

Higher energy ultrasound, for example, ultrasound which is generated by therapeutic ultrasound equipment, is generally capable of causing rupture of the vesicle species. In general, devices for therapeutic ultrasound employ from about 10 to about 100% duty cycles, depending on the area to be treated with the ultrasound. Areas of the body which are generally characterized by larger amounts of muscle mass, for example, backs and thighs, as well as highly vascularized tissues, such as heart tissue, may require a larger duty cycle, for example, up to about 100%.

The invention also includes methods of using the compounds of the invention in Magnetic Resonance Spectroscopy (MRS). MRS can be used to identify structures and/or compounds in the immediate vicinity of the compounds of the invention. By analysis of the resonance frequency of the surrounding atoms, which are slightly different in different compounds because of the electron shielding unique to each compound, different compounds are identifiable with MRS.

Accordingly, in another aspect of the invention MRS is used, with or without other imaging techniques. In one embodiment, the method is used to identify or locate soluble amyloid, fibrillar amyloid, and/or amyloid deposits.

The above methods can include the administration of additional agents or therapies, including agents that inhibit amyloid deposition that are not compounds of the invention. The administration may be staggered or contemporaneous with the administration of the compounds of the invention. Accordingly, the method can be used, e.g., to assess the efficacy of such additional compounds, by imaging a subject prior, concurrently or subsequent to the administration of the additional compound. The method can be used to determine how a therapeutic compound decreases or increases the rate of amyloid deposition or otherwise affects amyloids present in a subject or in a subject's body fluids.

The compounds of the present invention may be administered by any suitable route described herein, including, for example, parenterally (including subcutaneous, intramuscular, intravenous, intradermal and pulmonary), for imaging of internal organs, tissues, tumors, and the like. It will be appreciated that the route be selected depending on the organs or tissues to be imaged.

In one embodiment, the compound is administered alone. In another embodiment, it is administered as a pharmaceutical formulation comprising at least one compound of the invention and one or more pharmaceutically acceptable carriers, diluents or excipients as described herein. The formulation can optionally include delivery systems such as emulsions, liposomes and microparticles. The pharmaceutical formulation may optionally include other diagnostic or therapeutic agents, including other contrast agents, probes and/or diagnostic agents. The compounds of the present invention may also be presented for use in the form of veterinary formulations, which may be prepared, for example, by methods that are conventional in the art.

Dosages of the compounds of the invention can depend on the spin density, flow (diffusion and perfusion), susceptibility, and relaxivity (T1 and T2) of the compounds of the invention. Dosages of the compounds of the invention may be conveniently calculated in milligrams of ¹⁹F per kilogram of patient (abbreviated as mg ¹⁹F/kg). For example, for parenteral administration, typical dosages may be from about 50 to about 1000 mg ¹⁹F/kg, more preferably from about 100 to about 500 mg ¹⁹F/kg. The dosage may take into account other fluorinated compounds in the administered formula.

For methods of continuous administrations (e.g., intravenous), suitable rates of administration are known in the art. Typical rates of administration are about 0.5 to 5 mL of formulation per second, more preferably about 1-3 mL/s. Imaging may begin before or after commencing administration, continue during administration, and may continue after administration.

It will be appreciated that dosages, dosage volumes, formulation concentrations, rates of administration, and imaging protocols will be individualized to the particular patient and the examination sought, and may be determined by an experienced practitioner. Guidelines for selecting such parameters are known in the art. The Contrast Media Manual, (1992, R. W. Katzberg, Williams and Wilkins, Baltimore, Md.).

Synthesis of Compounds of the Invention

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here. Functional and structural equivalents of the compounds described herein and which have the same general properties, wherein one or more simple variations of substituents are made which do not adversely affect the essential nature or the utility of the compound are also included.

The compounds of the present invention may be readily prepared in accordance with the synthesis schemes and protocols described herein, as illustrated in the specific procedures provided. However, those skilled in the art will recognize that other synthetic pathways for forming the compounds of this invention may be used, and that the following is provided merely by way of example, and is not limiting to the present invention. See, e.g., “Comprehensive Organic Transformations” by R. Larock, VCH Publishers (1989). It will be further recognized that various protecting and deprotecting strategies will be employed that are standard in the art (See, e.g., “Protective Groups in Organic Synthesis” by Greene and Wuts). Those skilled in the relevant arts will recognize that the selection of any particular protecting group (e.g., amine and carboxyl protecting groups) will depend on the stability of the protected moiety with regards to the subsequent reaction conditions and will understand the appropriate selections.

Further illustrating the knowledge of those skilled in the art is the following sampling of the extensive chemical literature: “Chemistry of the Amino Acids” by J. P. Greenstein and M. Winitz, John Wiley & Sons, Inc., New York (1961); “Comprehensive Organic Transformations” by R. Larock, VCH Publishers (1989); T. D. Ocain, et al., J. Med. Chem. 31, 2193-99 (1988); E. M. Gordon, et al., J. Med. Chem. 31, 2199-10 (1988); “Practice of Peptide Synthesis” by M. Bodansky and A. Bodanszky, Springer-Verlag, New York (1984); “Protective Groups in Organic Synthesis” by T. Greene and P. Wuts (1991); “Asymmetric Synthesis: Construction of Chiral Molecules Using Amino Acids” by G. M. Coppola and H. F. Schuster, John Wiley & Sons, Inc., New York (1987); “The Chemical Synthesis of Peptides” by J. Jones, Oxford University Press, New York (1991); and “Introduction of Peptide Chemistry” by P. D. Bailey, John Wiley & Sons, Inc., New York (1992).

The synthesis of compounds of the invention is carried out in a solvent. Suitable solvents are liquids at ambient room temperature and pressure or remain in the liquid state under the temperature and pressure conditions used in the reaction. Useful solvents are not particularly restricted provided that they do not interfere with the reaction itself (that is, they preferably are inert solvents), and they dissolve a certain amount of the reactants. Depending on the circumstances, solvents may be distilled or degassed. Solvents may be, for example, aliphatic hydrocarbons (e.g., hexanes, heptanes, ligroin, petroleum ether, cyclohexane, or methylcyclohexane) or halogenated hydrocarbons (e.g., methylenechloride, chloroform, carbontetrachloride, dichloroethane, chlorobenzene, or dichlorobenzene); aromatic hydrocarbons (e.g., benzene, toluene, tetrahydronaphthalene, ethylbenzene, or xylene); ethers (e.g., diglyme, methyl-tert-butyl ether, methyl-tert-amyl ether, ethyl-tert-butyl ether, diethylether, diisopropylether, tetrahydrofuran or methyltetrahydrofurans, dioxane, dimethoxyethane, or diethyleneglycol dimethylether); nitriles (e.g., acetonitrile); ketones (e.g., acetone); esters (e.g., methyl acetate or ethyl acetate); and mixtures thereof.

In general, after completion of the reaction, the product is isolated from the reaction mixture according to standard techniques. For example, the solvent is removed by evaporation or filtration if the product is solid, optionally under reduced pressure. After the completion of the reaction, water may be added to the residue to make the aqueous layer acidic or basic and the precipitated compound filtered, although care should be exercised when handling water-sensitive compounds. Similarly, water may be added to the reaction mixture with a hydrophobic solvent to extract the target compound. The organic layer may be washed with water, dried over anhydrous magnesium sulphate or sodium sulphate, and the solvent is evaporated to obtain the target compound. The target compound thus obtained may be purified, if necessary, e.g., by recrystallization, reprecipitation, chromatography, or by converting it to a salt by addition of an acid or base.

The compounds of the invention may be supplied in a solution with an appropriate solvent or in a solvent-free form (e.g., lyophilized). In another aspect of the invention, the compounds and buffers necessary for carrying out the methods of the invention may be packaged as a kit, optionally including a container. The kit may be commercially used for treating or preventing amyloid-related disease according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The term “container” includes any receptacle for holding the therapeutic compound. For example, in one embodiment, the container is the packaging that contains the compound. In other embodiments, the container is not the packaging that contains the compound, i.e., the container is a receptacle, such as a box or vial that contains the packaged compound or unpackaged compound and the instructions for use of the compound. Moreover, packaging techniques are well known in the art. It should be understood that the instructions for use of the therapeutic compound may be contained on the packaging containing the therapeutic compound, and as such the instructions form an increased functional relationship to the packaged product.

Pharmaceutical Preparations

In another embodiment, the present invention relates to pharmaceutical compositions comprising agents according to any of the Formulae herein for the treatment of an amyloid-related disease, as well as methods of manufacturing such pharmaceutical compositions.

In general, the agents of the present invention may be prepared by the methods illustrated in the general reaction schemes as, for example, in the patents and patent applications referred to herein, or by modifications thereof, using readily available starting materials, reagents and conventional synthesis procedures. In these reactions, it is also possible to make use of variants which are in themselves known, but are not mentioned here. Functional and structural equivalents of the agents described herein and which have the same general properties, wherein one or more simple variations of substituents are made which do not adversely affect the essential nature or the utility of the agent are also included.

The agents of the invention may be supplied in a solution with an appropriate solvent or in a solvent-free form (e.g., lyophilized). In another aspect of the invention, the agents and buffers necessary for carrying out the methods of the invention may be packaged as a kit. The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The therapeutic agent may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

To administer the therapeutic agent by other than parenteral administration, it may be necessary to coat the agent with, or co-administer the agent with, a material to prevent its inactivation. For example, the therapeutic agent may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7, 27 (1984)).

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

Suitable pharmaceutically acceptable vehicles include, without limitation, any non-immunogenic pharmaceutical adjuvants suitable for oral, parenteral, nasal, mucosal, transdermal, intravascular (IV), intraarterial (IA), intramuscular (IM), and subcutaneous (SC) administration routes, such as phosphate buffer saline (PBS).

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

The therapeutic agent can be orally administered, for example, with an inert diluent or an assimilable edible carrier. The therapeutic agent and other ingredients may also be enclosed in a hard or soft shell gelatin capsule, compressed into tablets, or incorporated directly into the subject's diet. For oral therapeutic administration, the therapeutic agent may be incorporated with excipients and used in the form of ingestible tablets, buccal tablets, troches, capsules, elixirs, suspensions, syrups, wafers, and the like. The percentage of the therapeutic agent in the compositions and preparations may, of course, be varied. The amount of the therapeutic agent in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic agent calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a therapeutic agent for the treatment of amyloid-related disease in subjects.

The present invention therefore includes pharmaceutical formulations comprising the agents of the Formulae described herein, including pharmaceutically acceptable salts thereof, in pharmaceutically acceptable vehicles for aerosol, oral and parenteral administration. Also, the present invention includes such agents, or salts thereof, which have been lyophilized and which may be reconstituted to form pharmaceutically acceptable formulations for administration, as by intravenous, intramuscular, or subcutaneous injection. Administration may also be intradermal or transdermal.

In accordance with the present invention, an agent of the Formulae described herein, and pharmaceutically acceptable salts thereof, may be administered orally or through inhalation as a solid, or may be administered intramuscularly or intravenously as a solution, suspension or emulsion. Alternatively, the agents or salts may also be administered by inhalation, intravenously or intramuscularly as a liposomal suspension.

Pharmaceutical formulations are also provided which are suitable for administration as an aerosol, by inhalation. These formulations comprise a solution or suspension of the desired agent of any Formula herein, or a salt thereof, or a plurality of solid particles of the agent or salt. The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid agent of any Formula described herein, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol may be in the form of a liquid, the formulation will comprise a water-soluble agent of any Formula described herein, or a salt thereof, in a carrier which comprises water. A surfactant may be present which lowers the surface tension of the formulation sufficiently to result in the formation of droplets within the desired size range when subjected to nebulization.

Peroral compositions also include liquid solutions, emulsions, suspensions, and the like. The pharmaceutically acceptable vehicles suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or even from about 1% to about 5%, of an agent of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

In one embodiment, active agents are administered at a therapeutically effective dosage sufficient to inhibit amyloid deposition in a subject. A “therapeutically effective” dosage inhibits amyloid deposition by, for example, at least about 20%, or by at least about 40%, or even by at least about 60%, or by at least about 80% relative to untreated subjects. In the case of an Alzheimer's subject, a “therapeutically effective” dosage stabilizes cognitive function or prevents a further decrease in cognitive function (i.e., preventing, slowing, or stopping disease progression). The present invention accordingly provides therapeutic drugs. By “therapeutic” or “drug” is meant an agent having a beneficial ameliorative or prophylactic effect on a specific disease or condition in a living human or non-human animal.

In the case of AA or AL amyloidosis, the agent may improve or stabilize specific organ function. As an example, renal function may be stabilized or improved by 10% or greater, 20% or greater, 30% or greater, 40% or greater, 50% or greater, 60% or greater, 70% or greater, 80% or greater, or by greater than 90%.

In the case of IAPP, the agent may maintain or increase β-islet cell function, as determined by insulin concentration or the Pro-IAPP/IAPP ratio. In a further embodiment, the Pro-IAPP/IAPP ratio is increased by about 10% or greater, about 20% or greater, about 30% or greater, about 40% or greater, or by about 50%. In a further embodiment, the ratio is increased up to 50%. In addition, a therapeutically effective amount of the agent may be effective to improve glycemia or insulin levels.

In another embodiment, the active agents are administered at a therapeutically effective dosage sufficient to treat AA (secondary) amyloidosis and/or AL (primary) amyloidosis, by stabilizing renal function, decreasing proteinuria, increasing creatinine clearance (e.g., by at least 50% or greater or by at least 100% or greater), remission of chronic diarrhea, or by weight gain (e.g., 10% or greater). In addition, the agents may be administered at a therapeutically effective dosage sufficient to improve nephrotic syndrome.

Furthermore, active agents may be administered at a therapeutically effective dosage sufficient to decrease deposition in a subject of amyloid protein, e.g., Aβ40 or Aβ42. A therapeutically effective dosage decreases amyloid deposition by, for example, at least about 15%, or by at least about 40%, or even by at least 60%, or at least by about 80% relative to untreated subjects. Deposition of the amyloid protein may be decreased directly by, for example, inhibiting fibril formation, or indirectly by, for example, decreasing Aβ processing and, thus, decreasing the formation of fibrils in the brain and/or other locations.

In another embodiment, active agents are administered at a therapeutically effective dosage sufficient to increase or enhance amyloid protein, e.g., Aβ40 or Aβ42, in the blood, CSF, or plasma of a subject. A therapeutically effective dosage increases the concentration by, for example, at least about 15%, or by at least about 40%, or even by at least 60%, or at least by about 80% relative to untreated subjects.

In yet another embodiment, active agents are administered at a therapeutically effective dosage sufficient to maintain a subject's CDR rating at its base line rating or at 0. In another embodiment, the active agents are administered at a therapeutically effective dosage sufficient to decrease a subject's CDR rating by about 0.25 or more, about 0.5 or more, about 1.0 or more, about 1.5 or more, about 2.0 or more, about 2.5 or more, or about 3.0 or more. In another embodiment, the active agents are administered at a therapeutically effective dosage sufficient to reduce the rate of the increase of a subject's CDR rating as compared to historical or untreated controls. In another embodiment, the therapeutically effective dosage is sufficient to reduce the rate of increase of a subject's CDR rating (relative to untreated subjects) by about 5% or greater, about 10% or greater, about 20% or greater, about 25% or greater, about 30% or greater, about 40% or greater, about 50% or greater, about 60% or greater, about 70% or greater, about 80% or greater, about 90% or greater or about 100% or greater.

In yet another embodiment, active agents are administered at a therapeutically effective dosage sufficient to maintain a subject's score on the MMSE. In another embodiment, the active agents are administered at a therapeutically effective dosage sufficient to increase a subject's MMSE score by about 1, about 2, about 3, about 4, about 5, about 7.5, about 10, about 12.5, about 15, about 17.5, about 20, or about 25 points. In another embodiment, the active agents are administered at a therapeutically effective dosage sufficient to reduce the rate of the decrease of a subject's MMSE score as compared to historical controls. In another embodiment, the therapeutically effective dosage is sufficient to reduce the rate of decrease of a subject's MMSE score may be about 5% or less, about 10% or less, about 20% or less, about 25% or less, about 30% or less, about 40% or less, about 50% or less, about 60% or less, about 70% or less, about 80% or less, about 90% or less or about 100% or less, of the decrease of the historical or untreated controls.

In yet another embodiment, active agents are administered at a therapeutically effective dosage sufficient to maintain a subject's score on the DAD. In another embodiment, the active agents are administered at a therapeutically effective dosage sufficient to increase a subject's DAD score by about 1, about 2, about 3, about 4, about 5, about 10, about 15, about 20, about 25, about 30, about 40, or about 50 or more points. In another embodiment, the active agents are administered at a therapeutically effective dosage sufficient to reduce the rate of the decrease of a subject's DAD score as compared to historical controls. In another embodiment, the therapeutically effective dosage is sufficient to reduce the rate of decrease of a subject's DAD score may be about 5% or less, about 10% or less, about 20% or less, about 25% or less, about 30% or less, about 40% or less, about 50% or less, about 60% or less, about 70% or less, about 80% or less, about 90% or less or about 100% or less, of the decrease of the historical or untreated controls.

In yet another embodiment, active agents are administered at a therapeutically effective dosage sufficient to maintain a subject's score on the ADAS-Cog. In another embodiment, the active agents are administered at a therapeutically effective dosage sufficient to decrease a subject's ADAS-Cog score by about 2 points or greater, by about 3 points or greater, by about 4 points or greater, by about 5 points or greater, by about 7.5 points or greater, by about 10 points or greater, by about 12.5 points or greater, by about 15 points or greater, by about 17.5 points or greater, by about 20 points or greater, or by about 25 points or greater. In another embodiment, the active agents are administered at a therapeutically effective dosage sufficient to reduce the rate of the increase of a subject's ADAS-Cog scores as compared to historical or untreated controls. In another embodiment, the therapeutically effective dosage is sufficient to reduce the rate of increase of a subject's ADAS-Cog scores (relative to untreated subjects) by about 5% or greater, about 10% or greater, about 20% or greater, about 25% or greater, about 30% or greater, about 40% or greater, about 50% or greater, about 60% or greater, about 70% or greater, about 80% or greater, about 90% or greater or about 100% or greater.

In another embodiment, active agents are administered at a therapeutically effective dosage sufficient to decrease the ratio of Aβ42:Aβ40 in the CSF or plasma of a subject by about 15% or more, about 20% or more, about 25% or more, about 30% or more, about 35% or more, about 40% or more, about 45% or more, or about 50% or more.

In another embodiment, active agents are administered at a therapeutically effective dosage sufficient to lower levels of Aβ in the CSF or plasma of a subject by about 15% or more, about 25% or more, about 35% or more, about 45% or more, about 55% or more, about 75% or more, or about 95% or more.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50, and usually a larger therapeutic index is more efficacious. While agents that exhibit toxic side effects may be used, care should be taken to design a delivery system that targets such agents to the site of affected tissue in order to minimize potential damage to unaffected cells and, thereby, reduce side effects.

It is understood that appropriate doses depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the subject. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency. Such appropriate doses may be determined using the assays described herein. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, and any drug combination.

The ability of an agent to inhibit amyloid deposition can be evaluated in an animal model system that may be predictive of efficacy in inhibiting amyloid deposition in human diseases, such as a transgenic mouse expressing human APP or other relevant animal models where Aβ deposition is seen or for example in an animal model of AA amyloidosis. Likewise, the ability of an agent to prevent or reduce cognitive impairment in a model system may be indicative of efficacy in humans. Alternatively, the ability of an agent can be evaluated by examining the ability of the agent to inhibit amyloid fibril formation in vitro, e.g., using a fibrillogenesis assay such as that described herein, including a ThT, CD, or EM assay. Also the binding of an agent to amyloid fibrils may be measured using a MS assay as described herein. The ability of the agent to protect cells from amyloid induced toxicity may be determined in vitro using biochemical assays to determine percent cell death induced by amyloid protein. The ability of an agent to modulate renal function may also be evaluated in an appropriate animal model system.

The therapeutic agent of the invention may also be administered ex vivo to inhibit amyloid deposition or treat certain amyloid-related diseases, such as β₂M amyloidosis and other amyloidoses related to dialysis. Ex vivo administration of the therapeutic agents of the invention can be accomplished by contacting a body fluid (e.g., blood, plasma, etc.) with a therapeutic compound of the invention such that the therapeutic compound is capable of performing its intended function and administering the body fluid to the subject. The therapeutic compound of the invention may perform its function ex vivo (e.g., dialysis filter), in vivo (e.g., administered with the body fluid), or both. For example, a therapeutic compound of the invention may be used to reduce plasma β₂M levels and/or maintain β₂M in its soluble form ex vivo, in vivo, or both.

Prodrugs

The present invention is also related to prodrugs of the agents of the Formulae disclosed herein. Prodrugs are agents which are converted in vivo to active forms (see, e.g., R. B. Silverman, 1992, “The Organic Chemistry of Drug Design and Drug Action,” Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art (see, e.g., Berge, et al., “Pharmaceutical Salts”, J. Pharm. Sci. 66, 1-19 (1977)). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

Pharmaceutically Acceptable Salts

Certain embodiments of the present agents can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agents of the present invention. These salts can be prepared in situ during the final isolation and purification of the agents of the invention, or by separately reacting a purified agent of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethanesulfonate, and laurylsulphonate salts and the like. See, e.g., Berge et al., “Pharmaceutical Salts”, J. Pharm. Sci. 66, 1-19 (1977).

In other cases, the agents of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention.

These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

“Pharmaceutically acceptable salts” also includes, for example, derivatives of agents modified by making acid or base salts thereof, as described further below and elsewhere in the present application. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent agent formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid. Pharmaceutically acceptable salts may be synthesized from the parent agent which contains a basic or acidic moiety by conventional chemical methods. Generally, such salts may be prepared by reacting the free acid or base forms of these agents with a stoichiometric amount of the appropriate base or acid in water or in an organic solvent, or in a mixture of the two.

All acid, salt, base, and other ionic and non-ionic forms of the compounds described are included as compounds of the invention. For example, if a compound is shown as an acid herein, the salt forms of the compound are also included. Likewise, if a compound is shown as a salt, the acid and/or basic forms are also included.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. Further, this application is related to U.S. Ser. No. 10/871,514, entitled “Methods and Compositions for Treating Amyloid-Related Diseases” and filed on Jun. 18, 2004. The contents of all references, issued patents, and published patent applications cited throughout this application are hereby incorporated by reference. The invention is further illustrated by the following examples, which should not be construed as further limiting.

### EXAMPLES

Binding Assay

The test compounds were synthesized and screened by mass spectrometry (“MS”) assays. The MS assay gives data on the ability of compounds to bind to proteins, in this example, to β-amyloid.

In the MS assay for Aβ40, the sample was prepared as an aqueous solution (adding 20% ethanol if necessary to solubilize in water), 200 μM of a test compound and 20 μM of solubilized Aβ40, or 400 μM of a test compound and 40 μM of solubilized Aβ40. The pH value of the sample was adjusted to 7.4 (±0.2) by addition of 0.1% aqueous sodium hydroxide. The solution was then analyzed by electrospray ionization mass spectrometry using a Waters ZQ 4000 mass spectrometer. The sample was introduced by direct infusion at a flow-rate of 25 μL/min within 2 hr. after sample preparation. The source temperature was kept at 70° C. and the cone voltage was 20 V for all the analysis. Data were processed using Masslynx 3.5 software. The MS assay gives data on the ability of compounds to bind to soluble Aβ. It was found that (2,2,2-trifluoroethylamino)-propane sulfonic acid exhibited binding at 45-59% at a concentration of 400 μM and 20-44% at a concentration of 200 μM of test compound. The data from the assay is summarized in Table 2.

TABLE 2
Key to Table 2

| | Code | 400 μM | 200 μM |
|---|---|---|---|
| Strong Binding | *** | 90-100% | 60-100% |
| Moderate Binding | ** | 70-89% | 30-69% |
| Weak Binding | * | 45-59% | 20-44% |
| Little/no detectable binding | — | 20-39% | 20-39% |
| Not Tested | NT | | |

| | Aβ 40 |
|---|---|
| Structure | binding |
| 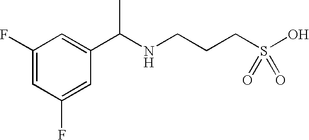 | *** |
| 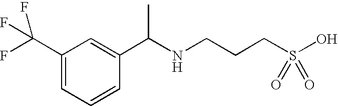 | *** |
| 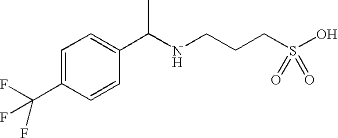 | *** |
| 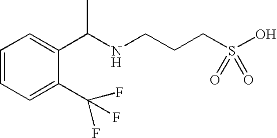 | *** |
| 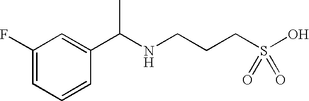 | ** |
| 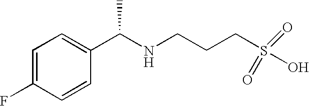 | NT |
| 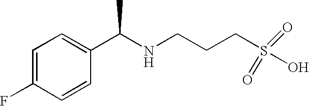 | NT |
| 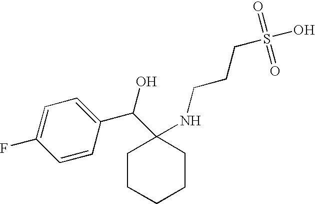 | — |
| 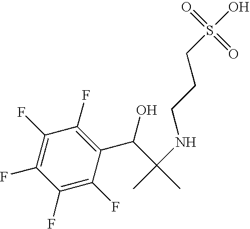 | *** |
| 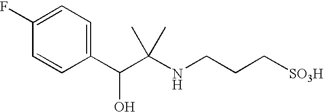 | NT |
| 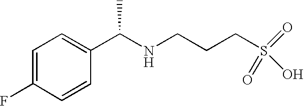 | NT |
| 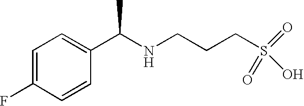 | *** |
| 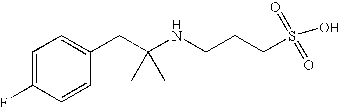 | NT |
| 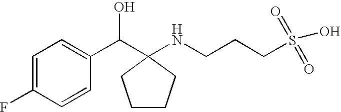 | ** |
| 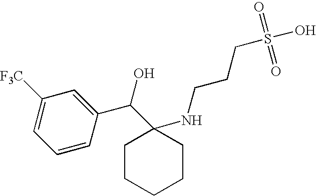 | NT |
| 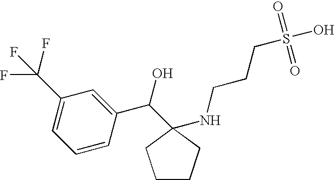 | ** |
| 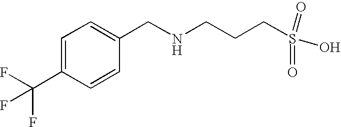 | *** |
| 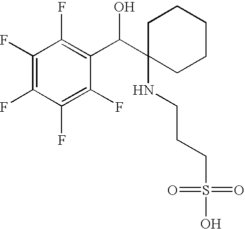 | * |
| 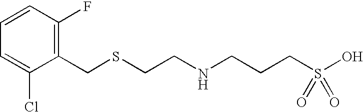 | ** |
| 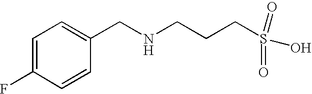 | ** |
| 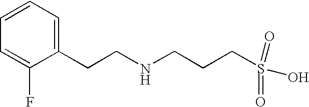 | ** |
| 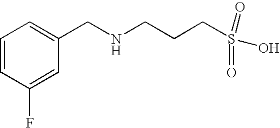 | * |
| 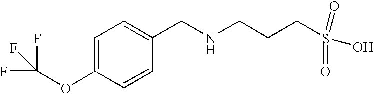 | *** |
| 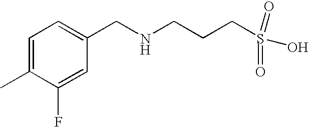 | *** |
| 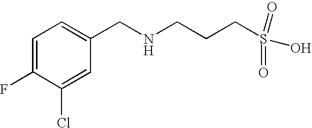 | *** |
| 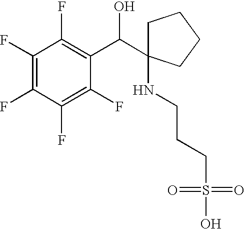 | ** |
| 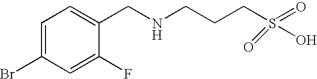 | *** |
| 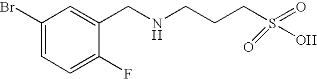 | ** |
| 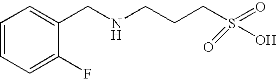 | *** |
| 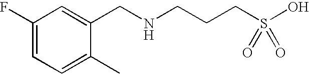 | *** |
| 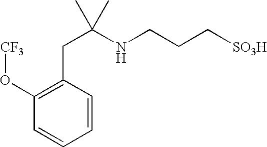 | ** |
| 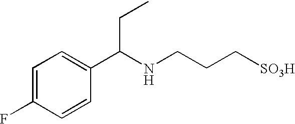 | *** |
| 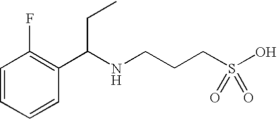 | ** |
| 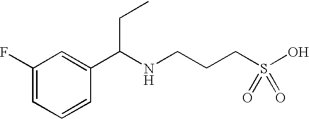 | *** |
| 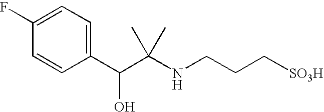 | *** |
| 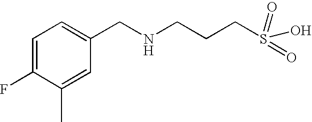 | * |
| 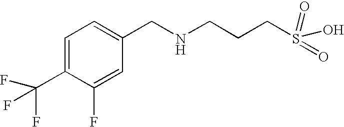 | *** |
| 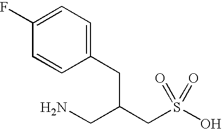 | *** |
| 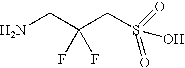 | * |
| 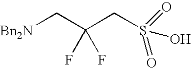 | * |
| 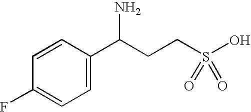 | *** |
| 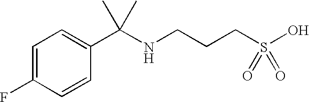 | *** |
| 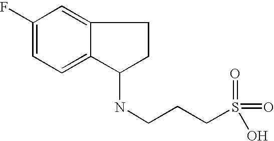 | *** |
| 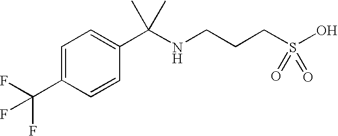 | *** |
| 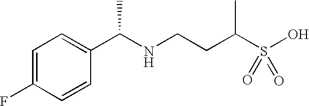 | * |
| 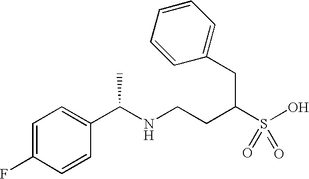 | NT |

ApoE/Aβ Solid-Phase Screening Assay

Nunc-Immuno Maxisorp 96-well microtiter plates are coated with 1 μM HFIP-disaggregated Aβ40 in 0.1 M NaHCO₃ (pH 9.6) for 2 hours and 15 minutes at 37° C. The plates are then washed twice in TBS (100 mM Tris-HCl, pH 7.5, 150 mM NaCl), and the wells are blocked with 1% fatty-acid free BSA in TBS overnight at 4° C. The compounds are prepared in either TBS (2 mM) or DMSO (10 mM). Recombinant ApoE (Fitzgerald Industries Int.) is prepared in 700 mM NH₄HCO₃ at a final concentration of 0.44 mg/mL. Purified ApoE (3.41 μg/mL) is pre-incubated in the presence of test compounds (200 μM) in 1% BSA/TBS in a 96-well transfer plate for one hour. The ApoE mixture is then added to the Aβcoated wells for an additional two hours with gentle shaking at 37° C. to allow ApoE/Aβ association. Plates are washed three times in TBS to remove excess ApoE and are incubated first with 0.125 μg/mL mouse monoclonal anti-ApoE antibody (BD Bioscience) for 1 hour. The plates are then washed and are incubated with 0.26 μg/mL horse-radish peroxidase conjugated goat anti-IgG antibody (Pierce) for 1 hour in 1% BSA/TBS-T (0.05% Tween-20). After washing, the wells are then incubated with Sure Blue™ TMB-1 peroxidase substrate (KPL) for 30 minutes. The reaction is stopped using 1N HCl. Absorbance values at 450 nm are measured using TECAN plate reader and reflect the amount of ApoE bound to Aβ in the wells. Data is expressed as a percentage of ApoE/Aβ complexes by arbitrarily setting ApoE alone at 100%.

Effects of Short Term Treatment in Adult Transgenic CRND8 Mice Overexpressing βAPP APP transgenic mice, TgCRND8, expressing the human amyloid precursor protein (hAPP) develop a pathology resembling Alzheimer's disease. In particular, high levels of Aβ40 and Aβ42 have been documented in the plasma and the brain of these animals at 8-9 weeks of age, followed by early accumulation of amyloid plaques similar to the senile plaques observed in AD patients. These animals also display progressive cognitive deficits that parallel the appearance of degenerative changes. See, e.g., (Chishti, et al., J. Biol. Chem. 276, 21562-70 (2001).

The short term therapeutic effect of compounds of the invention is studied. These compounds are administered over a 14 or 28 day period at the end of which the levels of Aβ peptides in the plasma and brain of TgCRND8 animals are determined.

Methods

Male and female APP transgenic mice are used in this example and given daily subcutaneous or oral administrations of one of a series of compounds for 14 or 28 days.

Baseline animals consist of TgCRND8 mice at 9±1 weeks of age. These mice are used to determine the Aβ levels in the plasma and brain of transgenic animals at the initiation of treatment.

Starting at 9 weeks of age (±1 week) animals receive daily administration of their respective treatment for a period of 14 or 28 days, at a dose of 250 mg/kg at 10 ml/kg or of vehicle only (water) or 1% methyl cellulose only. The route of administration may be oral or subcutaneous for water-soluble compounds and oral for compounds solubilized in methylcellulose 1% (MC 1%). At the end of the treatment periods, plasma and perfused brains are collected for quantification of soluble and insoluble Aβ levels.

TABLE 3
Test System

| | |
|---|---|
| Species: | Mouse |
| Strain: | TgCRND8.B6AF1/J (N₄) |
| Genotype: | hAPP +/− |
| Gender: | Male and Female |
| Age at Day 1: | 9 ± 1 weeks |
| Body Weight | 10 to 30 g |
| at Day 1: | |
| Number of | N = 20 |
| Animals/Group: | Baseline: 5 |
| Suppliers: | TgCRND8-2 founders were obtained from the Centre |
| | for Research in Neurodegenerative Diseases, |
| | University of Toronto. The hybrid B6AF1/J were |
| | obtained from Jackson Labs (Bar Harbor, Maine). |

Animal Health Monitoring

All animals are examined daily for signs of ill health when handled in the morning for their daily treatment and twice a day for mortality checks (once daily during weekends and holidays). Detailed examinations are performed on the treatment initiation, weekly during the study, and once before terminal procedures. More frequent observations are undertaken when considered appropriate. Death and all individual clinical signs are individually recorded. Individual body weights are recorded at randomization, once weekly during the study, and once before terminal procedures.

Sample Collection

At 9±1 weeks of age for the Baseline group, and at the end of the treatment period (14 or 28 days) for the treated Groups, at 24 hours after the last compound administration, animals are sacrificed and samples collected. An approximate blood volume of 500 μl is collected under general anesthesia from the orbital sinus and kept on ice until centrifugation at 4° C. at a minimum speed of 3,000 rpm for 10 minutes. Plasma samples are immediately frozen and stored at −80° C. pending analysis. After intracardiac saline perfusion, the brains are removed, frozen, and stored at −80° C. awaiting analysis.

Measurements of Aβ Levels

Brains are weighed frozen and homogenized with 4 volumes of ice cold 50 mM Tris-Cl pH 8.0 buffer with protease inhibitor cocktail (4 mL of buffer for 1 g of wet brain). Samples are spun at 15000 g for 20 minutes and the supernatants are transferred to fresh tubes. One hundred fifty (150) μl from each supernatant are mixed with 250 μl of 8M guanidine-HCL/50 mM Tris-HCL pH 8.0 (ratio of 0.6 vol supernatant: 1 vol 8M guanidium/Tris-HCL 50 mM pH8.0) and 400 μL 5 M guanidium/Tris-HCL 50 mM pH8.0 were added. The tubes are vortexed for 30 seconds and frozen at −80° C. In parallel, pellets are treated with 7 volumes of 5 M guanidine-HCL/50 mM Tris-HCL pH 8.0 (7 mL of guanidine for 1 g of wet brain), vortexed for 30 seconds and frozen at −80° C. Samples were thawed at room temperature, sonicated at 80° C. for 15 minutes and frozen again. This cycle is repeated 3 times to ensure homogeneity and samples are returned to −80° C. pending analysis.

Aβ levels are evaluated in plasma and brain samples by ELISA using Human Aβ40 and Aβ42 Fluorometric ELISA kits from Biosource (Cat. No. 89-344 and 89-348) according to manufacturer's recommended procedures. In short, samples are thawed at room temperature, sonicated for 5 minutes at 80° C. (sonication for brain homogenates; no sonication for plasma samples) and kept on ice. Aβ peptides are captured using 100 μl of the diluted samples to the plate and incubated without shaking at 4° C. overnight. The samples are aspirated and the wells are rinsed 4 times with wash buffer obtained from the Biosource ELISA kit. The anti-Aβ40 or anti-Aβ42 rabbit polyclonal antiserum (specific for the Aβ40 or Aβ42 peptide) is added (100 μl) and the plate is incubated at room temperature for 2 hours with shaking. The wells are aspirated and washed 4 times before adding 100 μl of the alkaline phosphatase labeled anti-rabbit antibody and incubating at room temperature for 2 hours with shaking. The plates are then rinsed 5 times and the fluorescent substrate (100 μl) is added to the plate. The plate is incubated for 35 minutes at room temperature and the plate is read using a titer plate reader at an excitation wavelength of 460 nm and emission at 560 nm.

Compounds are scored based on their ability to modulate levels of Aβ peptides in the plasma and the cerebral soluble/insoluble levels in the brain. Levels of Aβ observed in the plasma and brain of treated animals are normalized using values from vehicle-treated (water) or methylcellulose-treated control groups and ranked according to the strength of the pharmacological effect.

Effects Of Long Term Treatment in Adult Transgenic CRND8 Mice Overexpressing βAPP Transgenic mice, TgCRND8, as those used in the short term treatment, overexpress a human APP gene with the Swedish and Indiana mutations leading to the production of high levels of the amyloid peptides and to the development of an early-onset, aggressive development of brain amyloidosis. The high levels of Aβ peptides and the relative overabundance of Aβ₄₂ compared to Aβ₄₀ are believed to be associated with the severe and early degenerative pathology observed. The pattern of amyloid deposition, presence of dystrophic neuritis, and cognitive deficit has been well documented in this transgenic mouse line. The levels of Aβ peptides in the brain of these mice increase dramatically as the animals age. While the total amyloid peptide levels increase from ˜1.6×10⁵ pg/g of brain to ˜3.8×10⁶ between 9 and 17 weeks of age.

While the early deposition of amyloid in this model allows the rapid testing of compounds in a relatively short time frame, the aggressivity of this model and the high levels of Aβ peptides renders therapeutic assessment in the longer term a more difficult task.

The long-term therapeutic effects of compounds of the present invention on cerebral amyloid deposition and βamyloid (Aβ) levels in the plasma and in the brains of transgenic mice, TgCRND8, expressing the human amyloid precursor protein (hAPP) is studied. These compounds are administered over a 4, 8 or 16 week period at the end of which the levels of Aβ peptides in the plasma and brain of TgCRND8 animals are determined. The goal of this study is to evaluate the efficacy of the compounds at modulating the progression of the amyloidogenic process in the brain and in the plasma of a transgenic mouse model of Alzheimer's disease (AD).

Methods

The mice to be used in the study consist of animals bearing one copy of the hAPP gene (+/−) derived from backcrosses from TgCRND8 with B6AF1/J hybrid animals.

Male and female transgenic mice are given daily subcutaneous or oral administrations of the appropriate compounds for 4, 8 or 16 weeks.

Baseline animals consist of 9±1 week old naive TgCRND8.B6AF1/J animals. These mice are used to determine the extent of cerebral amyloid deposits and Aβ levels in the plasma and brain of naive transgenic animals at the initiation of treatment.

Starting at 9 weeks of age (±1 week) animals receive daily administration of their respective treatment for a period of 4, 8 or 16 weeks, at a dose of 30 or 100 mg/kg at 10 ml/kg. The route of administration is subcutaneous or oral for water-soluble compounds and oral for compounds solubilized in methylcellulose 1% (MC 1%). At the end of the treatment periods, plasma and perfused brains are collected for quantification of Aβ levels. The steady state pharmacokinetic profile is evaluated based on plasma samples.

Animal health is monitored, samples are collected and Aβ levels are measured as described above in the short term treatment study. Compounds are scored based on their ability to modulate levels of Aβ peptides in the plasma and the cerebral soluble/insoluble levels in the brain. Levels of Aβ observed in the plasma and brain of treated animals are compared to that of vehicle-treated (water) or methylcellulose-treated control groups and ranked according to the strength of the pharmacological effect.

After 4 weeks of treatment, there was a reduction in both soluble and insoluble Aβ 42 levels in the brains of mice treated with (S)-3-[1-(4-fluorophenyl)ethylamino]-1-propanesulfonic acid.

Evaluation of Compounds Binding to NAC Peptide by Mass Spectrometry

Recent findings have demonstrated that a high percentage of Alzheimer Disease (AD) patients also form Lewy bodies, most abundantly in the amygdala (Hamilton. 2000. Brain Pathol, 10:378; Mukaetova-Ladinska, et al. 2000. J Neuropathol Exp Neurol 59:408). Interestingly, the highly hydrophobic non-amyloid component (NAC) region of α-synuclein has also been described as the second most abundant component of amyloid plaques in the brain of AD patients. Alpha-synuclein has been shown to form fibrils in vitro. Furthermore, it binds to Aβ and promotes its aggregation (Yoshimoto, et al. 1995. Proc Natl Acad Sci USA 92:9141). It was in fact originally identified as the precursor of the non-amyloid beta (Aβ) component (NAD) of AD plaques (Ueda, et al. 1993. Proc Natl Acad Sci USA 90:11282; Iwai. 2000. Biochem Biophys Acta 1502:95; Masliah, et al. 1996. Am J Pathol 148:201). NAC is a 35 amino acid long peptide with highly hydrophobic stretches which can self-aggregate and form fibrils in vitro. Moreover, these fibrils can efficiently seed the formation of Aβ fibrils in vitro (Han, et al. 1995. Chem Biol. 2: 163-169; Iwai, et al. 1995. Biochemistry 34:10139). Not to be limited by theory, it is thought that it is through the NAC domain that alpha-synuclein retains its fibrillogenic properties.

The ability of the compounds of the present invention to bind to NAC peptide in aqueous solution is evaluated. The binding ability correlates to the intensities of the peptide-compound complex peaks observed by the Electrospray Mass Spectrum. Millipore distilled deionized water is used to prepare all aqueous solutions. For pH determination, a Beckman Φ36 pH meter fitted with a Corning Semi-Micro Combination pH Electrode is employed.

Mass Spectrometry

Mass spectrometric analysis is performed using a Waters ZQ 4000 mass spectrometer equipped with a Waters 2795 sample manager. MassLynx 4.0 (earlier by MassLynx 3.5) is used for data processing and analysis. Test compounds are mixed with disaggregated peptides in aqueous media (6.6% EtOH) at a 5:1 ratio (20 μM NAC:100 μM of test compound or 40 μM NAC:200 μM of test compound). The pH of the mixture is adjusted to 7.4 (±0.2) using 0.1% NaOH (3-5 μL). Periodically, NAC peptide solution at 20 μM or 40 μM is also prepared in the same fashion and run as control. The spectra are obtained by introducing the solutions to the electrospray source by direct infusion using a syringe pump at a flow rate of 25 μl/min, and scanning from 100 to 2100 Da in the positive mode. The scan time is 0.9 second per scan with an inter-scan delay of 0.1 second and the run time is 5 minutes for each sample. All the mass spectra are sum of 300 scans. The desolvation and source temperature is 70° C. and the cone and capillary voltage are maintained at 20 V and 3.2 kV respectively.

The total area under the peaks for the bound NAC-compound complex divided by total area under the peaks for unbound NAC is determined for each compound tested.

In Vivo Imaging Employing Compounds of the Invention

Compounds of the invention will be suspended in a pharmaceutically acceptable carrier such as sterile water or physiological saline. Fluorine (¹⁹F) Magnetic Resonance Imaging will be carried out using standard procedures and commercially available equipment. Fluorine imaging may be performed, e.g., with the following parameters: TR=1 second, TE=18 milliseconds, image data matrix=64×64, NEX=32, FOV=128 nm. Fluorine MRI scans will be performed on subjects before and after administration of the contrast agent. Proton MRI may be used to provide anatomic markers for assessment of the fluorine images. Imaging agent dosages may be calculated as described in the following example.

Imaging Agent Dosage Calculations

Imaging dosages will depend on the solubility of the compound(s) administered, the route of administration, the carrier vehicle, the site to be imaged and the method of imaging. Dosages of ¹⁹F containing imaging agents may be conveniently calculated in milligrams of ¹⁹F per kilogram of patient (abbreviated as mg ¹⁹F/kg). For example, for parenteral administration, typical dosages may be from about 100 mg ¹⁹F/kg to about 500 mg ¹⁹F/kg. For the ¹⁹F MRI agent 3-[(2,2,2-trifluoroethyl)amino]-1-propanesulfonic acid (CF₃CH₂NH(CH₂)₃SO₃H), which has a molecular weight of 221.20 and which contains 3 fluorine atoms), the fluorine content is 25.77% by weight. For a typical 70 kg patient, a dosage of from about 7 g to about 35 g of ¹⁹F, or from about 27 to 136 g of this agent may be suitable.

Synthesis of Compounds of the Invention

### Preparation of 3-[(2,2,2-trifluoroethyl)amino]-1-propanesulfonic acid

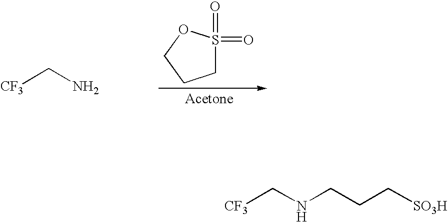

To a solution of 2,2,2-trifluroethylamine (1.00 g, 10.0 mmol) in acetone (13 mL) was slowly added 1,3-propanesultone (1.20 g, 9.6 mmol). The mixture was stirred at 35° C. for 7 h. The solvent was evaporated under reduced pressure. The residual material was suspended in acetone (20 mL), collected by filtration, washed with acetone (2×10 mL), and dried in a vacuum oven (50° C.) to give the title compound. Yield: 4%. ¹H NMR (DMSO, 500 MHz) δ ppm 9.72 (s (broad), 1H), 4.07 (m, 2H), 3.16 (t, 2H, J=6.5 Hz), 2.65 (t, 2H, J=6.5 Hz), 1.99 (m, 2H). ¹³C NMR (DMSO, 125 MHz) δ ppm 50.04, 48.85, 46.90, 22.17. ES-MS 219 (M−1).
General Experimental Procedure for Parallel Synthesis

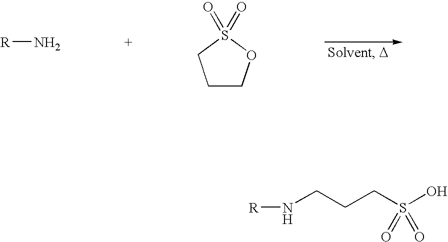

The amines (1 g each) were diluted with acetonitrile (2 mL) and were transferred into reaction tubes. To the reaction tubes was added a solution of 1,3-propanesultone (1M, 1 equiv.). The tubes were placed in Radley carrousel (12 position) and were heated under reflux for 4 hours, and then cooled to room temperature. The solids were collected by filtration, rinsed with acetone (2×5 mL) then dried for 18 hours in a vacuum oven at 60° C. The solvent was removed under reduced pressure and was replaced by toluene.

### Preparation of 3-[1-(3,5-difluorophenyl)ethylamino]-1-propanesulfonic acid

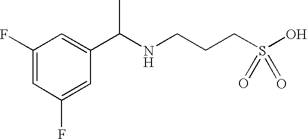

3-[1-(3,5-difluorophenyl)ethylamino]-1-propanesulfonic acid was prepared from (RS)-1-(3,5-difluorophenyl)ethylamine, white solid, yield 1.2 g, 68%. ¹H NMR (500 MHz, DMSO-d6) δ 1.52 (d, J=6.8 Hz, 3H), 1.95 (qt, J=6.6 Hz, 2H), 2.64 (t, J=6.3 Hz, 2H), 2.82-2.85 (m, 1H), 3.02 (br s, 1H), 4.44 (br d, J=6.3 Hz, 1H), 7.25-7.35 (m, 3H), 9.10 (br s, 1H), 9.32 (br s, 1H); ¹³C NMR (125 MHz, DMSO-d6) 18.8, 21.8, 45.2, 49.1, 55.9, 104.5 (t, J=25 Hz) 111.2 (d, J=27 Hz), 141.3, 161.5 (d, J=13.4 Hz), 163.5 (d, J=13.4 Hz); ¹⁹F NMR (282 MHz, DMSO-d6) δ −108.9 (t, J=8.8 Hz, 2F); ES-MS 278 (M−H)

### Preparation of 3-{1-[3-(trifluoromethyl)phenyl]ethylamino}-1-propanesulfonic acid

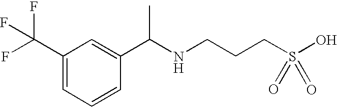

3-{1-[3-(trifluoromethyl)phenyl]ethylamino}-1-propanesulfonic acid was prepared from (RS)-1-[3-(trifluoromethyl)phenyl]-ethylamine, white solid; yield 1.29 g, 78%. ¹H NMR (500 MHz, DMSO-d6) δ 1.55 (d, J=6.8 Hz, 3H), 1.95 (qt, J=6.6 Hz, 2H), 2.63 (t, J=6.6 Hz, 2H), 2.82-2.84 (m, 1H), 3.07 (br s, 1H), 4.53 (br d, J=5.9 Hz, 1H), 7.71 (t, J=7.8 Hz, 1H), 7.81 (t, J=9.3 Hz, 2H), 7.90 (s, 1H), 9.11 (br s, 1H), 9.31 (br s, 1H); ¹³C NMR (125 MHz, DMSO-d6) 18.8, 21.9, 45.2, 49.2, 56.2, 124.0 (q, J=272 Hz), 124.5 (d, J=2.9 Hz), 125.7 (d, J=3.8 Hz), 129.5 (q, J=32 Hz), 130.2, 131.8, 138.7; ¹⁹F NMR (282 MHz, DMSO-d6) δ −61.7 (s, 3F); ES-MS 310 (M−H)

### Preparation of 3-{1-[4-(trifluoromethyl)phenyl]ethylamino}-1-propanesulfonic acid

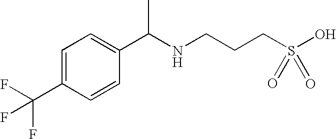

3-{1-[4-(trifluoromethyl)phenyl]ethylamino}-1-propanesulfonic acid was prepared from (RS)-1-[4-(trifluoromethyl)phenyl]-ethylamine, white solid; yield 1.49 g, 91%. ¹H NMR (500 MHz, DMSO-d6) δ 1.54 (d, J=6.8 Hz, 3H), 1.95 (qt, J=6.6 Hz, 2H), 2.63 (t, J=10.0 Hz, 2H), 2.80-2.85 (m, 1H), 3.03-3.082 (m, 1H), 4.51 (q, J=6.5 Hz, 1H), 7.72 (d, J=7.9 Hz, 1H), 7.85 (d, J=7.8 Hz, 2H), 9.17 (br s, 1H), 9.34 (br s, 1H); ¹³C NMR (125 MHz, DMSO-d6) 19.0, 21.9, 45.3, 49.2, 56.3, 124.0 (q, J=272 Hz), 125.9 (d, J=3.8 Hz), 128.6, 129.4 (q, J=32 Hz), 141.9; ¹⁹F NMR (282 MHz, DMSO-d6) δ −61.8 (s, 3F); ES-MS 310 (M−H)

### Preparation of 3-[1-(3-fluorophenyl)ethylamino]-1-propanesulfonic acid

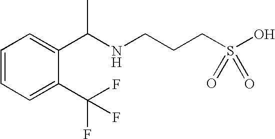

3-[1-(3-fluorophenyl)ethylamino]-1-propanesulfonic acid was prepared from 1-(3-fluorophenyl)ethylamine, white solid; yield 1.60 g, 85%. ¹H NMR (500 MHz, DMSO-d6) δ 1.53 (d, J=6.8 Hz, 3H), 1.95 (qt, J=6.6 Hz, 2H), 2.63 (t, J=6.6 Hz, 2H), 2.81 (qt, J=6.5 Hz, 1H), 3.03 (qt, J=6.3 Hz, 1H), 4.41 (q, J=6.5 Hz, 1H), 7.24-7.28 (m, 1H), 7.33-7.39 (m, 2H), 7.49-7.53 (m, 1H), 9.08 (br s, 1H), 9.24 (br s, 1H); ¹³C NMR (125 MHz, DMSO-d6) 19.0, 21.8, 45.1, 49.1, 56.2, 114.5 (d, J=22 Hz), 114.8 (d, J=21 Hz), 123.8, 131.1 (d, J=8.6 Hz), 139.9 (d, J=6.7 Hz), 161.2, 163.1; ¹⁹F NMR (282 MHz, DMSO-d6) δ −112.4-−112.5 (m, 1F); ES-MS 260 (M−H)

### Preparation of 3-{1-[2-(trifluoromethyl)phenyl]ethylamino}-1-propanesulfonic acid

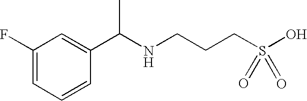

3-{1-[2-(trifluoromethyl)phenyl]ethylamino}-1-propanesulfonic acid was prepared from (RS)-1-[2-(trifluoromethyl)phenyl]-ethylamine, white solid; yield 0.76 g, 46%. ¹H NMR (500 MHz, DMSO-d6) δ 1.56 (d, J=6.8 Hz, 3H), 1.97 (qt, J=6.6 Hz, 2H), 2.63-2.66 (m, 2H), 2.82 (br s, 1H), 3.06 (br s, 1H), 4.50 (br s, 1H), 7.65 (t, J=7.6 Hz, 1H), 7.83-7.8 (m, 2H), 7.93 (d, J=7.8 Hz, 1H), 9.33 (br s, 1H), 9.60 (br s, 1H); ¹³C NMR (125 MHz, DMSO-d6) 20.5, 21.9, 45.7, 49.1, 53.2, 123.9 (q, J=274 Hz), 126.2 (q, J=5.8 Hz), 126.8 (q, J=30 Hz), 127.7, 129.4, 133.8, 136.1; ¹⁹F NMR (282 MHz, DMSO-d6) δ −57.6 (s, 3F); ES-MS 310 (M−H)

### Preparation of (R)-3-[1-(4-fluorophenyl)ethylamino]-1-propanesulfonic acid

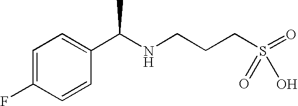

To a solution of (R)-(+)-1-(4-fluorophenyl)ethylamine (5.09 g, 36.6 mmol) in pinacolone (24 mL) and toluene (24 mL) was added 1,3-propane sultone (4.25 g, 34.8 mmol). The solution was stirred at reflux for 4 h. The reaction mixture was cooled to room temperature. The solid was collected by filtration and was washed with acetone (2×25 mL). The solid was suspended in ethanol (60 mL). The suspension was stirred at reflux for 1 h. The mixture was cooled to room temperature, the solid material was collected by filtration, washed with acetone (2×25 mL) and dried in a vacuum oven at 50° C., affording the title compound, 7.33 g (81%). ¹H NMR (D₂O, 500 MHz) δ ppm 7.36 (dd, 2H, J=2.4 Hz, 5.4 Hz), 7.10 (t, 2H, J=9.0 Hz), 4.32 (q, 1H, J=6.8 Hz), 3.00 (m, 1H), 2.84 (m, 1H), 2.79 (t, 2H, J=7.3 Hz), 1.94 (m, 2H), 1.54 (d, 3H, J=6.8 Hz); ¹³C (D₂O, 125 MHz) δ ppm 164.20, 162.23, 131.70, 129.93, 129.87, 116.42, 116.25, 57.87, 48.02, 44.35, 21.48, 18.19; ¹⁹F NMR (282 MHz, D₂O) δ −115.1 (m, 1F); [α]_{D}=+16.6° (c=0.0028 in water), ES-MS 260 (M−1).

### Preparation of (S)-3-[1-(4-fluorophenyl)ethylamino]-1-propanesulfonic acid

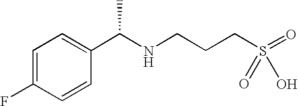

To a solution of(S)-(−)-1-(4-fluorophenyl)ethylamine (5.36 g, 38.5 mmol) in pinacolone (24 mL) and toluene (24 mL) was added 1,3-propane sultone (4.48 g, 36.7 mmol). The solution was stirred at reflux for 4 h. The reaction mixture was cooled to room temperature. The solid was collected by filtration and was washed with acetone (2×25 mL). The solid was suspended in EtOH (60 mL). The suspension was stirred at reflux for 1 h. The mixture was cooled to room temperature, the solid was collected by filtration, washed with acetone (2×25 mL) and dried in a vacuum oven at 50° C., affording the title compound, 7.85 g (91%). ¹H NMR (D₂O, 500 MHz) δ ppm 7.36 (dd, 2H, J=2.4 Hz, 5.4 Hz), 7.10 (t, 2H, J=9.0 Hz), 4.31 (q, 1H, J=6.8 Hz), 3.00 (m, 1H), 2.84 (m, 1H), 2.79 (t, 2H, J=7.3 Hz), 1.94 (m, 2H), 1.54 (d, 3H, J=6.8 Hz). ¹³C (D₂O, 125 MHz) δ ppm 164.23, 162.26, 131.73, 129.96, 129.89, 116.45, 116.27, 57.90, 48.04, 44.36, 21.49, 18.20. ¹⁹F NMR (282 MHz, D₂O) δ −112.9 (hept, J=4.6 Hz, 1F); [α]_{D}=−12.7° (c=0.0045 in water), ES-MS 260 (M−1).

### Preparation of 3-{1-[1-hydroxyl-(4-fluorobenzyl)]cyclohexyl}amino-1-propanesulfonic acid

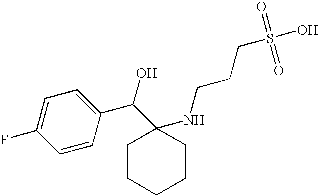

To a cooled solution of sodium methoxide (0.5 M in methanol, 80 mL, 40 mmol) was added nitrocyclohexane (4.7 mL, 38.7 mmol) via syringe over a 10 minutes period. The reaction mixture was stirred at room temperature for 30 minutes. The mixture was then cooled and 4-fluorobenzaldehyde (4.1 mL, 38.7 mmol) was added. The reaction mixture was stirred at room temperature overnight. The mixture was neutralized with Amberlite IR-120 (strongly acidic). The resin was removed by filtration and washed with MeOH (2×20 mL). The filtrate was evaporated. The resulting oil was purified by flash chromatography: 98% Hexanes/EtOAc to 95% Hexanes/EtOAc, affording the desired nitro compound (1.02 g, 11%).

To a solution of the nitro compound (1.02 g, 4.0 mmol)) in methanol (15 mL) was added 6M HCl (4 mL). After cooling to 5° C., zinc powder (1.28 g, 20.0 mmol) was added. The suspension was stirred at room temperature overnight. The mixture was filtered on a celite pad. The filter cake was washed with methanol (2×15 mL). The combined filtrates were evaporated under reduced pressure to afford the corresponding amine. The amine (0.813 g, 91%) was used without further purification.

To a solution of amine (0.813 g, 3.6 mmol) in pinacolone (5 mL) and toluene (5 mL) was added 1,3-propane sultone (415 mg, 3.4 mmol). The solution was stirred at reflux overnight. The reaction mixture was cooled to room temperature. The solid was collected by filtration, was washed with acetone (2×10 mL). The solid was suspended in EtOH (20 mL). The suspension was stirred at reflux for 1 hour. The mixture was cooled to room temperature, the solid material was collected by filtration, washed with acetone (2×10 mL) and dried in a vacuum oven at 50° C., affording the title compound, 0.690 g (61%). ¹H NMR (DMSO, 500 MHz) δ ppm 8.19 (s (broad), 1H), 7.39 (m, 2H), 7.19 (t, 2H, J=8.8 Hz), 6.32 (d, 1H, J=4.1 Hz), 4.82 (d, 1H, J=4.1 Hz), 3.17 (m, 2H), 2.68 (m, 2H), 2.07 (m, 2H), 1.87 (m, 2H), 1.53 (m, 5H), 1.18 (m, 2H), 0.92 (m, 1H); ¹³C (DMSO, 125 MHz) δ ppm 163.81, 160.59, 136.73, 130.82, 130.72, 115.44, 115.17, 72.63, 64.62, 50.27, 41.66, 28.29, 27.88, 25.47, 22.98, 20.16, 19.87; ¹⁹F NMR (282 MHz, DMSO-d6) δ −115.1 (s, 1F); ES-MS 344 (M−1).

### Preparation of 3-(2-hydroxy-1,1-dimethyl-2-(pentafluorophenyl)ethylamino)-1-propanesulfonic acid

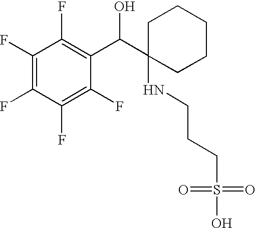

To a cooled solution of sodium methoxide (0.5 M in MeOH, 20 mmol), 2-nitropropane (4.9 mL, 51 mmol) was added via syringe over 10 minutes. The reaction mixture was stirred at room temperature for 30 minutes and recooled before pentafluorobenzaldehyde (10 g, 51 mmol) was added. The reaction mixture was stirred at room temperature over the weekend. The mixture was neutralized with Amberlite IR-120 (strongly acidic). The resin was removed by filtration and washed with MeOH (2×20 mL). The filtrate was evaporated. The resulting oil was purified by flash chromatography: 98% Hexanes/EtOAc to 95% Hexanes/EtOAc, affording the desired nitro compound (4.92 g, 34%).

To a solution of the nitro compound (4.92 g, 17.2 mmol) in MeOH (25 mL) was added 6M HCl (25 mL). After cooling to 5° C., zinc powder (8.2 g, 125 mmol) was added. The suspension was stirred at room temperature overnight. The mixture was filtered on a celite pad. The filter cake was washed with MeOH (2×20 mL). The combined filtrates were evaporated under reduced pressure. The residue was dissolved in EtOAc (40 mL). The mixture was extracted with 5% NaOH (3×40 mL). The organic phase was dried with Na₂SO₄, filtered, evaporated and dried in vacuo to afford the corresponding amine. The amine (3.14 g, 72%) was used without further purification.

To a solution of amine (1.50 g, 5.9 mmol) in pinacolone (5 mL) and toluene (5 mL) was added 1,3-propane sultone (683 mg, 5.6 mmol). The solution was stirred at reflux overnight. The reaction mixture was cooled to room temperature. The solid was collected by filtration, was washed with acetone (2×10 mL) and dried in a vacuum oven at 50° C., affording the title compound, 0.286 g (13%). ¹H NMR (DMSO, 500 MHz) δ ppm 8.69 (s (broad), 1H), 6.81 (s (broad), 1H), 5.09 (s, 1H), 3.11 (m, 2H), 2.63 (m, 2H), 1.99 (m, 2H), 1.24 (s, 3H), 1.12 (s, 3H); ¹³C (DMSO, 125 MHz) δ ppm 146.13, 144.25, 141.80, 139.79, 138.77, 136.83, 114.18, 68.92, 62.78, 49.88, 22.93, 19.95, 19.04; ¹⁹F NMR (282 MHz, DMSO-d6) δ −162.5 (s, 2F), −155.2 (t, J=21.6 Hz, 1F), −139.9 (br s, 1F), −137.4 (br s, 1F); ES-MS 376 (M−1).

### Preparation of 3-[1,1-dimethyl-2-(4-fluorophenyl)-2-hydroxyethylamino]-1-propanesulfonic acid

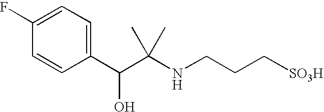

A mixture of 2-niropropane (2.3 g, 26.21 mmol), aldehyde (2.5 g, 20.16 mmol) and sodium methoxide (0.5 M, 112 mL) was stirred for 2 days. The reaction mixture was acidified with HCl (1M) and diluted with EtOAc. The organic layer was washed with HCl (1M) dried (Na₂SO₄) and concentrated. The crude was purified by column using Hexanes:EtOAc 90:10 to afford 1.3 g (23%) of the Henry-aldol product as a colorless solid.

To a stirred solution of the obtained nitro compound (1 g, 4.32 mmol) in EtOAc (40 mL) was added a spatula of Pd/C. The suspension was hydrogenated under one atmospheric pressure of hydrogen for 15 hours (TLC indicates complete consumption of the starting material) then filtered on celite and concentrated under reduced pressure. The corresponding amine was used as such in the next step.

To a stirred solution of the amine (680 mg, 3.71 mmol) in THF (10 mL) was added 1,3-propane sultone (453 mg, 3.71 mmol). The reaction mixture was stirred at reflux for 4 hours then cooled to room temperature. The solid was collected by filtration and was washed with THF. The solid was suspended in EtOH (10 mL) and stirred at reflux for 1 hour. The suspension was then cooled to room temperature. The solid was collected by filtration, washed with ethanol and dried under high vacuum to afford the title compound, 850 mg (75%). ¹H NMR (500 MHz, DMSO-d₆) δ 1.13 (s, 6H), 2.00 (m, 2H), 2.66 (dd, J=7.0 & 7.0 Hz, 2H), 2.75 (s, 2H), 3.10 (dd, J=7.0 & 7.0 Hz, 2H), 6.72 (d, J=8.3 Hz, 2H), 7.00 (d, J=8.3 Hz, 2H), 8.60 (bs, 2H), 9.36 (s, 1H); ¹³NMR (125 MHz, DMSO-d₆) δ 23.1, 41.2, 43.2, 49.8, 59.4, 115.7, 125.8, 132.3, 157.1; ES-MS 304 (M−1).

### Preparation of 3-({2-hydroxy-1,1-dimethyl-2-(pentafluorophenyl)ethyl]amino)-1-propanesulfonic acid

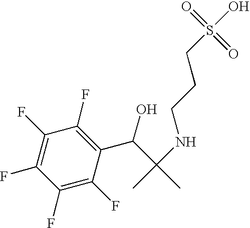

To a cooled solution of sodium methoxide (0.5 M in MeOH, 20 mLl) was added via syringe over a 10 minute period 2-nitropropane (4.9 mL, 51 mmol). The reaction mixture was stirred at room temperature for 30 minutes and recooled before Pentafluorobenzaldehyde (10 g, 51 mmol) was added. The reaction mixture was stirred at room temperature over the weekend. The mixture was neutralized with Amberlite IR-120 (strongly acidic). The resin was removed by filtration and washed with MeOH (2×20 mL). The filtrate was evaporated. The resulting oil was purified by flash chromatography: 98% Hexanes/EtOAc to 95% Hexanes/EtOAc, affording the desired nitro compound (4.92 g, 34%).

To a solution of the nitro compound (4.92 g, 17.2 mmol)) in MeOH (25 mL) was added 6M HCl (25 mL). After cooling to 5° C., zinc powder (8.2 g, 125 mmol) was added. The suspension was stirred at room temperature overnight. The mixture was filtered on a celite pad. The filter cake was washed with MeOH (2×20 mL). The combined filtrates were evaporated under reduced pressure. The residue was dissolved in EtOAc (40 mL). The mixture was extracted with 5% NaOH (3×40 mL). The organic phase was dried with Na₂SO₄, filtered, evaporated and dried in vacuo to afford the corresponding amine. The amine (3.14 g, 72%) was used without further purification.

To a solution of amine (1.50 g, 5.9 mmol) in pinacolone (5 mL) and toluene (5 mL) was added 1,3-propane sultone (683 mg, 5.6 mmol). The solution was stirred at reflux overnight. The reaction mixture was cooled to room temperature. The solid was collected by filtration, was washed with acetone (2×10 mL) and dried in a vacuum oven at 50° C., affording the title compound, 0.286 g (13%). ¹H NMR (DMSO, 500 MHz) δ ppm 8.69 (s (broad), 1H), 6.81 (s (broad), 1H), 5.09 (s, 1H), 3.11 (m, 2H), 2.63 (m, 2H), 1.99 (m, 2H), 1.24 (s, 3H), 1.12 (s, 3H). ¹³C (DMSO, 125 MHz) δ ppm 146.13, 144.25, 141.80, 139.79, 138.77, 136.83, 114.18, 68.92, 62.78, 49.88, 22.93, 19.95, 19.04; ¹⁹F NMR (282 MHz, DMSO-d6) δ ppm −162.5 (s, 2F), −155.2 (t, J=21.6 Hz, 1F), −139.9 (br s, 1F), −137.4 (br s, 1F); ES-MS 376 (M−1).

### Preparation of 3-{[2-(4-fluorophenyl)-1,1-dimethylethyl]amino}-1-propanesulfonic acid

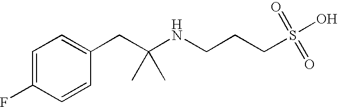

To a solution of 1-(4-fluorophenyl)-2-methyl-2-propylamine (2.30 g, 13.8 mmol) in acetone (8 mL) and toluene (8 mL) was added 1,3-propane sultone (1.61 g, 13.2 mmol). The solution was stirred at reflux for 8 hours. The reaction mixture was cooled to room temperature. The solid material was collected by filtration and washed with acetone (2×20 mL). The solid was suspended in EtOH (30 mL). The suspension was stirred at reflux for 1 hour. The mixture was cooled to room temperature. The solid material was collected by filtration, washed with acetone (2×20 mL) and dried in a vacuum oven at 50° C., affording the title compound, 2.97 g (78%). ¹H NMR (DMSO, 300 MHz) δ ppm 8.61 (s (broad), 1H), 7.26 (m, 2H), 7.16 (t, 2H, J=8.9 Hz), 3.13 (m, 1H), 2.87 (s, 2H), 2.67 (t, 2H, J=6.7 Hz), 1.98 (m, 2H), 1.15 (s, 6H). ¹³C (DMSO, 75 MHz) δ ppm 163.52, 160.31, 133.22, 133.11, 131.83, 115.88, 115.59, 59.35, 50.03, 43.17, 41.53, 23.46, 23.39. ¹⁹F (DMSO, 282 MHz) −114.46. ES-MS 288 (M−1).

### Preparation of 3-({1-[(4-fluorophenyl)(hydroxy)methyl]cyclopentyl}amino)-1-propanesulfonic acid

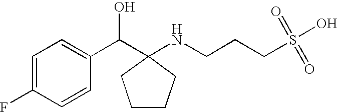

To a cooled solution of sodium methoxide (0.5 M in MeOH, 20 mL) was added via syringe over a 10 minutes period 2-nitrocyclopentane (2.99 g, 26 mmol). The reaction mixture was stirred at room temperature for 30 minutes and recooled before 4-fluorobenzaldehyde (2.7 mL, 26 mmol) was added. The reaction mixture was stirred at room temperature overnight. The mixture was neutralized with Amberlite IR-120 (strongly acidic). The resin was removed by filtration and washed with MeOH (2×20 mL). The filtrate was evaporated. The resulting oil was purified by flash chromatography: 98% Hexanes/EtOAc to 93% Hexanes/EtOAc, affording the desired nitro compound (1.7 g, 27%).

To a solution of the nitro compound (1.70 g, 7.1 mmol)) in MeOH (15 mL) was added 6M HCl (8 mL). After cooling to 5° C., zinc powder (2.35 g, 36.0 mmol) was added. The suspension was stirred at 0-5° C. for 30 minutes and at room temperature overnight. The mixture was filtered on a celite pad. The filter cake was washed with MeOH (2×10 mL). The combined filtrates were evaporated under reduced pressure. The residue was dissolved in EtOAc (35 mL). The mixture was extracted with 5% NaOH (1×35 mL). The aqueous phase was extracted with EtOAc (2×35 mL). The combined organic extracts were dried with Na₂SO₄, filtered, evaporated and dried in vacuo to afford the corresponding amine. The amine (1.31 g, 88%) was used without further purification.

To a solution of amine (1.31 g, 6.3 mmol) in acetonitrile (6 mL) and acetone (8 mL) was added 1,3-propane sultone (731 mg, 6.0 mmol). The solution was stirred at reflux overnight. The reaction mixture was cooled to room temperature. The solid material was collected by filtration, was washed with acetone (2×15 mL). The solid was suspended in EtOH (20 mL). The suspension was stirred at reflux for 1 hour. The mixture was cooled to room temperature. The white solid was filtered, washed with acetone (2×15 mL) and dried in a vacuum oven at 50° C., affording the title compound, 1.33 g (67%). ¹H NMR (DMSO, 500 MHz) δ ppm 8.55 (s (broad), 2H), 7.50 (m, 2H), 7.19 (t, 1H, J=8.8 Hz), 6.39 (d, 1H, J=4.1 Hz), 4.89 (d, 1H, J=3.8 Hz), 3.21 (m, 1H), 3.11 (m, 1H), 2.64 (m, 2H), 2.05 (m, 3H), 1.78 (m, 2H), 1.52 (m, 3H), 0.86 (m, 1H), 0.70 (m, 1H). ¹³C (DMSO, 125 MHz) δ ppm 163.43, 161.48, 136.67, 130.71, 130.65, 115.54, 115.37, 110.00, 72.69, 71.80, 49.88, 42.55, 31.65, 31.15, 25.03, 24.91, 22.98. ¹⁹F (DMSO, 282 MHz) −115.02. ES-MS 330 (M−1).

### Preparation of 3-[(1-{hydroxy[3-(trifluoromethyl)phenyl]methyl}cyclohexyl)amino]-1-propane-sulfonic acid

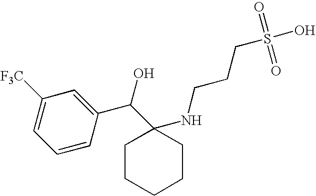

To a cooled solution of sodium methoxide (0.5 M in MeOH, 20 mL) was added via syringe over a 10 minute period nitrocyclohexane (4.7 mL, 38.7 mmol). The reaction mixture was stirred at room temperature for 30 minutes and recooled before α,α,α-trifluoro-m-tolualdehyde (5.1 mL, 38.7 mmol) was added. The reaction mixture was stirred at room temperature overnight. The mixture was neutralized with Amberlite IR-120 (strongly acidic). The resin was removed by filtration and washed with MeOH (2×20 mL). The filtrate was evaporated. The resulting oil was purified by flash chromatography: 98% Hexanes/EtOAc to 93% Hexanes/EtOAc, affording the desired nitro compound (4.47 g, 38%).

To a solution of the nitro compound (4.47 g, 14.8 mmol)) in MeOH (30 mL) was added 6M HCl (16 mL). After cooling to 5° C., zinc powder (4.82 g, 73.7 mmol) was added. The suspension was stirred at 0-5° C. for 30 minutes and at room temperature overnight. The mixture was filtered on a celite pad. The filter cake was washed with MeOH (2×20 mL). The combined filtrates were evaporated under reduced pressure. The residue was dissolved in EtOAc (80 mL). The mixture was extracted with 5% NaOH (1×80 mL). The aqueous phase was extracted with EtOAc (2×80 mL). The combined organic extracts were dried with Na₂SO₄, filtered, evaporated and dried in vacuo to afford the corresponding amine. The amine (3.98 g, 99%) was used without further purification.

To a solution of amine (3.98 g, 14.6 mmol) in toluene (9 mL) and acetone (9 mL) was added 1,3-propane sultone (1.62 g, 13.2 mmol). The solution was stirred at reflux over the weekend. The reaction mixture was cooled to room temperature. The solid material was collected by filtration, was washed with acetone (2×15 mL). The solid was suspended in EtOH (25 mL). The suspension was stirred at reflux for 1 h. The mixture was cooled to room temperature. The white solid was filtered, washed with acetone (2×15 mL) and dried in a vacuum oven (50° C.), affording the title compound, 3.73 g (71%). ¹H NMR (DMSO, 500 MHz) δ ppm 8.35 (s (broad), 1H), 8.24 (s (broad), 7.69 (m, 3H), 7.61 (t, 1H, J=7.6 Hz), 6.46 (d, 1H, J=4.4 Hz), 4.96 (d, 1H, J=3.9 Hz), 3.24 (m, 1H), 3.13 (m, 1H), 2.68 (m, 2H), 2.08 (m, 2H), 1.93 (m, 2H), 1.50 (m, 5H), 1.14 (m, 2H), 0.91 (m, 1H). ¹³C (DMSO, 125 MHz) δ ppm 142.22, 133.12, 129.66, 129.37, 129.13, 125.31, 72.52, 64.43, 50.11, 41.54, 28.02, 27.60, 25.10, 22.65, 19.79, 19.50. ¹⁹F (DMSO, 282 MHz) δ ppm −61.62. ES-MS 394 (M−1).

### Preparation of 3-[(1-{hydroxy[3-(trifluoromethyl)phenyl]methyl}cyclopentyl)amino]-1-propanesulfonic acid

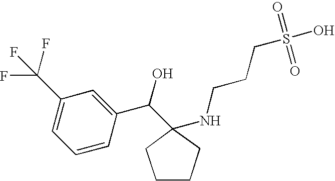

To a cooled solution of sodium methoxide (0.5 M in MeOH, 20 mL) was added via syringe over a 10 minutes period 2-nitrocyclopentane (3.00 g, 26 mmol). The reaction mixture was stirred at room temperature for 30 minutes and recooled before α,α,α-trifluoro-m-tolualdehyde (3.5 mL, 26 mmol) was added. The reaction mixture was stirred at room temperature overnight. The mixture was neutralized with Amberlite IR-120 (strongly acidic). The resin was removed by filtration and washed with MeOH (2×15 mL). The filtrate was evaporated. The product crystallized while drying on the pump. The solid material was filtered, washed with 98% Hexanes/EtOAc (2×15 mL) and dried in vacuo, affording the desired nitro compound (3.18 g, 42%).

To a solution of the nitro compound (3.18 g, 11.0 mmol)) in MeOH (20 mL) was added 6M HCl (14 mL). After cooling to 5° C., zinc powder (3.59 g, 55.02 mmol) was added. The suspension was stirred at 0-5° C. for 30 minutes and at room temperature overnight. The mixture was filtered on a celite pad. The filter cake was washed with MeOH (2×20 mL). The combined filtrates were evaporated under reduced pressure. The residue was dissolved in EtOAc (80 mL). The mixture was extracted with 5% NaOH (1×80 mL). The aqueous phase was extracted with EtOAc (2×80 mL). The combined organic extracts were dried with Na₂SO₄, filtered, evaporated and dried in vacuo to afford the corresponding amine. The amine (2.48 g, 89%) was used without further purification.

To a solution of amine (2.48 g, 9.8 mmol) in acetone (5 mL) and toluene (5 mL) was added 1,3-propane sultone (1.09 g, 8.9 mmol). The solution was stirred at reflux overnight. The reaction mixture was cooled to room temperature. The solid material was collected by filtration, was washed with acetone (2×10 mL). The solid was suspended in EtOH (15 mL). The suspension was stirred at reflux for 1 hour. The mixture was cooled to room temperature. The white solid was filtered, washed with acetone (2×15 mL) and dried in a vacuum oven at 50° C., affording the title compound, 0.448 g (12%). ¹H NMR (DMSO, 300 MHz) δ ppm 8.60 (s (broad), 2H), 7.79 (m, 2H), 7.69 (m, 1H), 7.60 (t, 1H, J=7.3 Hz), 6.49 (d, 1H, J=3.8 Hz), 5.03 (d, 1H, J=2.9 Hz), 3.17 (m, 2H), 2.64 (t, 2H, J=6.9 Hz), 2.13 (m, 1H), 2.04 (m, 2H), 1.80 (m, 2H), 1.56 (m, 3H), 0.82 (m, 1H), 0.64 (m, 1H). ¹³C (DMSO, 75 MHz) δ ppm 141.98, 132.83, 129.74, 129.51, 129.10, 126.64, 125.35, 125.10, 72.74, 71.75, 50.06, 42.84, 31.91, 31.58, 25.23, 25.03, 23.30. ¹⁹F (DMSO, 282 MHz) δ ppm −61.69. ES-MS 80 (M−1).

### Preparation of 3-{[4-(trifluoromethyl)benzyl]amino}-1-propanesulfonic acid

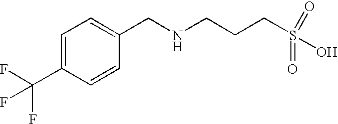

To a solution of 4-(trifluoromethyl)benzyl]amine (4.95 g, 28.3 mmol) in acetone (16 mL) and toluene (16 mL) was added 1,3-propane sultone (3.29 g, 26.9 mmol). The solution was stirred at reflux for 4 hours. The reaction mixture was cooled to room temperature. The solid material was collected by filtration and washed with acetone (2×15 mL). The solid was suspended in EtOH (30 mL). The suspension was stirred at reflux for 1 hour. The mixture was cooled to room temperature, the solid material was collected by filtration, washed with acetone (2×15 mL) and dried in a vacuum oven at 50° C., affording the title compound, 3.87 g (48%). ¹H NMR (D₂O, 300 MHz) δ ppm 7.63, (d, 2H, J=8.2 Hz), 7.47 (d, 2H, J=7.9 Hz), 4.17 (s, 2H), 3.09 (t, 2H, J=7.9 Hz), 2.83 (t, 2H, J=7.3 Hz), 1.99 (m, 2H). ¹³C (D₂O, 75 MHz) δ ppm 134.81, 131.45, 131.19, 130.94, 130.77, 130.68, 130.50, 130.40, 130.26, 130.05, 127.28, 126.31, 126.28, 125.12, 122.96, 120.80, 50.59, 49.00, 47.99, 46.13, 21.38. ¹⁹F (D₂O, 282 MHz) δ ppm −63.43. ES-MS 296 (M−1).

### Preparation of 3-[2-(2-chloro-6-fluorobenzylthio)ethylamino]-1-propanesulfonic acid

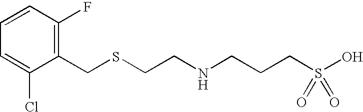

A solution of 1,3-propane sultone (1.0 M in MeCN, 4.55 mL) was added to a solution of 2-(2-chloro-6-fluorobenzylthio)ethylamine (1 g, 4.55 mmol) in MeCN (10 mL, solution was filtered). The mixture was heated to 85° C. for 3 hours on the Radley caroussel. The suspension was cooled to room temperature. The solid was collected by suction filtration, rinsed with acetone (2×5 mL). The solid was dried 18 hours at 60° C. in the vacuum oven. The title compound was obtained as a fine white solid (1.20 g, 3.51 mmol, 77%). ¹H NMR (500 MHz, DMSO-d6) δ 1.94 (qt, J=6.7 Hz, 2H), 2.61 (t, J=6.6 Hz, 2H), 2.77 (t, J=7.6 Hz, 2H), 3.09 (t, J=6.8 Hz, 2H), 3.16 (t, J=7.6 Hz, 2H), 3.92 (d, J=0.98 Hz, 2H), 7.26-7.29 (m, 1H), 7.36-7.41 (m, 2H), 8.64 (br s, 2H); ¹³C NMR (125 MHz, DMSO-d6) δ 21.8, 26.0, 25.4, 29.0, 45.9, 46.8, 48.8, 114.6 (d, J=23 Hz), 124.4 (d, J=18.2 Hz), 125.8, 129.9 (d, J=9.6 Hz), 134.2, 160.6 (d, J=248 Hz) ¹⁹F NMR (282 MHz, DMSO-d6) δ −113.24-−1113.46 (m, 1F); ES-MS 340, 342 (M−H).

### Preparation of 3-[(4-fluorobenzyl)amino]-1-propanesulfonic acid

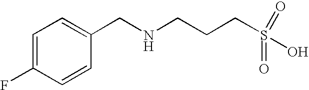

To a solution of 4-Fluorobenzy]amine (1.0 g, 8.0 mmol) in Acetone (5 mL) and Toluene (5 mL) was added 1,3-propane sultone solution (1.5 M in THF, 5.1 mL, 7.6 mmol). The solution was stirred at reflux for 4 hours. The reaction mixture was cooled to room temperature. The solid material was collected by filtration and washed with acetone (2×10 mL) and dried in a vacuum oven at 50° C., affording the title compound, 0.616 g (31%). ¹H NMR (D₂O, 300 MHz) δ ppm 7.34, (m, 2H), 7.06 (m, 2H), 4.11 (s, 2H), 3.09 (t, 2H, J=7.8 Hz), 2.83 (t, 2H, J=7.3 Hz), 2.00 (m, 2H). ¹³C (D₂O, 75 MHz) δ ppm 164.74, 161.49, 132.09, 131.97, 126.72, 116.33, 116.04, 50.70, 48.21, 46.01, 21.70. ¹⁹F (D₂O, 282 MHz) δ ppm −112.88. ES-MS 246 (M−1).

### Preparation of 3-{[2-(2-fluorophenyl)ethyl]amino}-1-propanesulfonic acid

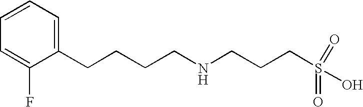

To a solution of 2-Fluorophenethylamine (1.0 g, 7.2 mmol) in acetone (4.5 mL) and toluene (4.5 mL) was added 1,3-propane sultone solution (1.5 M in THF, 4.6 mL, 6.8 mmol). The solution was stirred at reflux for 4 hours. The reaction mixture was cooled to room temperature. The solid material was collected by filtration and washed with acetone (2×10 mL) and dried in a vacuum oven at 50° C., affording the title compound, 0.774 g (41%). ¹H NMR (D₂O, 300 MHz) δ ppm 7.18, (m, 2H), 7.01 (m, 2H), 3.19 (t, 2H, J=7.3 Hz), 3.06 (t, 2H, J=7.9 Hz), 2.92 (t, 2H, J=7.5 Hz), 2.83 (t, 2H, J=7.3 Hz), 1.96 (m, 2H). ¹³C (D₂O, 75 MHz) δ ppm 162.63, 159.41, 131.74, 131.68, 129.77, 129.67, 125.35, 124.20, 116.17, 115.90, 49.68, 47.59, 47.16, 26.14, 22.85. ¹⁹F (D₂O, 282 MHz) δ ppm −119.48. ES-MS 260 (M−1).

### Preparation of 3-[(3-fluorobenzyl)amino]-1-propanesulfonic acid

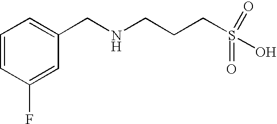

To a solution of 3-Fluorobenzy]amine (1.0 g, 8.0 mmol) in acetone (5 mL) and toluene (5 mL) was added 1,3-propane sultone solution (1.5 M in THF, 5.1 mL, 7.6 mmol). The solution was stirred at reflux for 4 hours. The reaction mixture was cooled to room temperature. The solid material was collected by filtration and washed with acetone (2×10 mL) and dried in a vacuum oven at 50° C., affording the title compound, 0.616 g (31%). ¹H NMR (D₂O, 300 MHz) δ ppm 7.33, (m, 1H), 7.13 (d, 1H, J=7.3 Hz), 7.08 (m, 2H), 4.12 (s, 2H), 3.09 (t, 2H, J=7.6 Hz), 2.84 (t, 2H, J=6.8 Hz), 1.99 (m, 2H). ¹³C (D₂O, 75 MHz) δ ppm 164.14, 160.91, 132.89, 131.22, 131.12, 125.71, 116.78, 116.51, 50.80, 48.18, 46.18, 21.69. ¹⁹F (DMSO, 282 MHz) δ ppm −113.03. ES-MS 246 (M−1).

### Preparation of 3-{[4-(trifluoromethoxy)benzyl]amino}-1-propanesulfonic acid

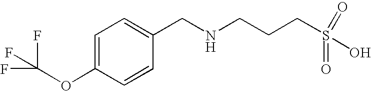

To a solution of 4-(Trifluoromethoxy)benzylamine (1.0 g, 5.2 mmol) in Acetone (3.25 mL) and Toluene (3.25 mL) was added 1,3-propane sultone solution (1.5 M in THF, 3.3 mL, 5.0 mmol). The solution was stirred at reflux overnight. The reaction mixture was cooled to room temperature. The solid material was collected by filtration and washed with acetone (2×10 mL) and dried in a vacuum oven at 50° C., affording the title compound, 0.432 g (27%). ¹H NMR (D₂O, 300 MHz) δ ppm 7.38, (d, 2H, J=8.4 Hz), 7.23 (d, 2H, J=8.4 Hz), 4.11 (s, 2H), 3.07 (t, 2H, J=7.8 Hz), 2.83 (t, 2H, J=7.3 Hz), 1.99 (m, 2H). ¹³C (D₂O, 75 MHz) δ ppm 149.67, 131.68, 129.51, 125.38, 121.97, 121.67, 118.59, 115.20, 50.56, 48.20, 46.12, 21.69. ¹⁹F (D₂O, 282 MHz) δ ppm −58.63. ES-MS 246 (M−1).

### Preparation of 3-[(3-fluoro-4-methylbenzyl)amino]-1-propanesulfonic acid

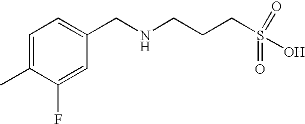

To a solution of 3-fluoro-4-methylbenzylamine (1.0 g, 7.2 mmol) in acetone (4.5 mL) and toluene (4.5 mL) was added 1,3-propane sultone solution (1.5 M in THF, 4.6 mL, 6.8 mmol). The solution was stirred at reflux for 4 hours. The reaction mixture was cooled to room temperature. The solid material was collected by filtration and washed with acetone (2×10 mL) and dried in a vacuum oven at 50° C., affording the title compound, 0.601 g (32%). ¹H NMR (D₂O, 300 MHz) δ ppm 7.20, (t, 1H, J=7.8 Hz), 7.02 (m, 2H), 4.06 (s, 2H), 3.07 (t, 2H, J=7.8 Hz), 2.84 (t, 2H, J=7.4 Hz), 2.21 (s, 3H), 1.98 (m, 2H). ¹³C (D₂O, 75 MHz) δ ppm 162.13, 160.19, 132.50, 132.46, 130.05, 129.98, 127.05, 126.91, 125.57, 125.55, 116.38, 116.19, 50.49, 48.00, 45.85, 21.38, 13.62. ¹⁹F (DMSO, 282 MHz) δ ppm −117.42. ES-MS 260 (M−1).

### Preparation of 3-[(3-chloro-4-fluorobenzyl)amino]-1-propanesulfonic acid

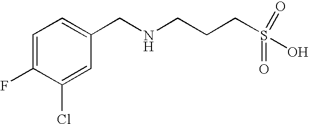

To a solution of 3-chloro-4-fluorobenzy]amine (1.0 g, 7.8 mmol) in Acetone (2 mL) and Toluene (2 mL) was added 1,3-propane sultone solution (1.5 M in THF, 4.0 mL, 6.0 mmol). The solution was stirred at reflux for 4 hours. The reaction mixture was cooled to room temperature. The solid material was collected by filtration and washed with acetone (2×5 mL) and dried in a vacuum oven at 50° C., affording the title compound, 0.041 g (2%). ¹H NMR (D₂O, 300 MHz) δ ppm 7.44 (dd, 1H, J=2.2 Hz, 6.9 Hz), 7.25 (m, 1H), 7.15 (t, 2H, J=8.6 Hz), 4.07 (s, 2H), 3.07 (t, 2H, J=7.8 Hz), 2.83 (t, 2H, J=7.5 Hz), 1.98 (m, 2H). ¹³C (D₂O, 75 MHz) δ ppm 160.07, 156.77, 132.18, 130.43, 130.22, 127.90, 120.94, 117.58, 117.29, 50.20, 48.17, 46.09, 21.67. ¹⁹F (D₂O, 282 MHz) δ ppm −115.33. ES-MS 280 (M−1).

### Preparation of 3-({1-[hydroxy(pentafluorophenyl)methyl]cyclopentyl}amino)-1-propanesulfonic acid

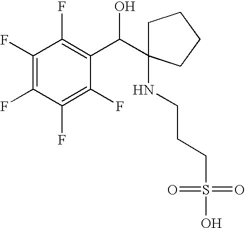

To a cooled solution of sodium methoxide (0.5 M in MeOH, 15 mL) was added via syringe over a 10 minute period 2-nitrocyclopentane (5.0 mL, 40.9 mmol). The reaction mixture was stirred at room temperature for 30 minutes and recooled before pentafluorobenzaldehyde (2.1 mL, 17.4 mmol) was added. The reaction mixture was stirred at room temperature overnight. The solvent was evaporated. The product was purified by flash chromatography (98% Hexanes/EtOAc to 90% Hex/EtOAc), affording the desired nitro compound (1.47 g, 26%).

To a solution of the nitro compound (1.47 g, 4.5 mmol)) in MeOH (10 mL) was added 6M HCl (6 mL). After cooling to 5° C., zinc powder (1.47 g, 22.5 mmol) was added. The suspension was stirred at 0-5° C. for 30 minutes and at room temperature overnight. The mixture was filtered on a celite pad. The filter cake was washed with MeOH (2×10 mL). The combined filtrates were evaporated under reduced pressure. The residue was dissolved in EtOAc (35 mL). The mixture was extracted with 5% NaOH (1×35 mL). The aqueous phase was extracted with EtOAc (2×35 mL). The combined organic extracts were dried with Na₂SO₄, filtered, evaporated and dried in vacuo to afford the corresponding amine. The amine (1.23 g, 92%) was used without further purification.

To a solution of amine (1.23 g, 4.2 mmol) in Pinacolone (5 mL) and Toluene (5 mL) was added 1,3-propane sultone (1.71 g, 14.0 mmol). The solution was stirred at reflux overnight. The reaction mixture was cooled to room temperature. The solid material was collected by filtration, was washed with acetone (2×10 mL). The solid was suspended in EtOH (15 mL). The suspension was stirred at reflux for 1 hour. The mixture was cooled to room temperature. The white solid was filtered, washed with acetone (2×10 mL) and dried in a vacuum oven at 50° C., affording the title compound, 0.527 g (33%). ¹H NMR (DMSO, 500 MHz) δ ppm 8.70 (s (broad), 1H), 6.82 (s, 1H), 5.21 (s, 1H), 3.18 (m, 2H), 2.65 (t, 2H, J=6.6 Hz), 2.02 (m, 3H), 1.88 (m, 1H), 1.79 (m, 1H), 1.63 (m, 3H), 1.32 (m, 2H). ¹³C (DMSO, 125 MHz) δ ppm 146.31, 144.36, 141.83, 138.74, 136.79, 114.20, 73.26, 67.66, 50.03, 42.93, 31.72, 31.09, 24.65, 22.77. ¹⁹F (DMSO, 282 MHz) δ ppm −138.48, −154.30, −154.38, −154.46, −161.96. ES-MS 402 (M−1).

### Preparation of 3-[(4-bromo-2-fluorobenzyl)amino]-1-propanesulfonic acid

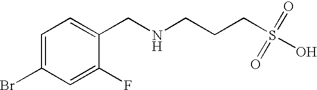

4-Bromo-2-fluorobenzylamine hydrochloride (5.0 g, 20.8 mmol) was treated with a saturated solution of K₂CO₃ (80 mL) and EtOAc (3×80 mL) was added. The organic extracts were combined, dried with Na₂SO₄, filtered, evaporated under reduced pressure and dried in vacuo.

To a solution of 4-Bromo-2-fluorobenzylamine (20.8 mmol) in 50% Pinacolone/Toluene (25 mL) was added 1,3-propane sultone solution (2.3 g, 18.9 mmol). The solution was stirred at reflux for 4 hours. The reaction mixture was cooled to room temperature. The solid material was collected by filtration and washed with acetone (2×20 mL). The solid was suspended in EtOH (40 mL). The suspension was stirred at reflux for 1 hour. The mixture was cooled to room temperature, the solid material was collected by filtration, washed with acetone (2×20 mL) and dried in a vacuum oven (50° C.), affording the title compound, 4.42 g (65%). ¹H NMR (D₂O, 500 MHz) δ ppm 7.35 (m, 2H), 7.26 (t, 2H, J=7.8 Hz), 4.16 (m, 2H), 3.11 (t, 2H, J=7.8 Hz), 2.85 (t, 2H, J=7.6 Hz), 2.00 (m, 2H). ¹³C (D₂O, 125 MHz) δ ppm 161.98, 159.97, 133.37, 128.50, 124.39, 124.31, 119.79, 119.60, 117.32, 117.19, 48.00, 46.15, 44.40, 21.34. ¹⁹F (D₂O, 282 MHz) δ ppm −114.64. ES-MS 325 (M−1).

### Preparation of 3-[(5-bromo-2-fluorobenzyl)amino]-1-propanesulfonic acid

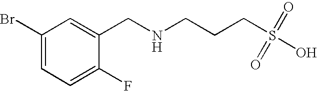

5-Bromo-2-fluorobenzylamine hydrochloride (5.0 g, 20.8 mmol) was treated with a saturated solution of K₂CO₃ (60 mL) and EtOAc (3×60 mL) was added. The organic extracts were combined, dried with Na₂SO₄, filtered, evaporated under reduced pressure and dried in vacuo.

To a solution of 4-bromo-2-fluorobenzylamine (20.8 mmol) in 25% Toluene/Acetonitrile (25 mL) was added 1,3-propane sultone solution (2.3 g, 18.9 mmol). The solution was stirred at reflux for 4 hours. The reaction mixture was cooled to room temperature. The solid material was collected by filtration and washed with acetone (2×20 mL). The solid was suspended in EtOH (40 mL). The suspension was stirred at reflux for 1 hour. The mixture was cooled to room temperature, the solid material was collected by filtration, washed with acetone (2×20 mL) and dried in a vacuum oven (50° C.), affording the title compound, 5.55 g (65%). ¹H NMR (D₂O, 500 MHz) δ ppm 7.52 (m, 2H), 7.04 (t, 2H, J=8.5 Hz), 4.16 (m, 2H), 3.12 (t, 2H, J=7.8 Hz), 2.85 (t, 2H, J=7.3 Hz), 2.01 (m, 2H). ¹³C (D₂O, 125 MHz) δ ppm 161.49 159.52, 135.29, 135.22, 134.79, 120.20, 120.07, 118.17, 117.98, 116.80, 48.04, 46.23, 44.45, 21.39. ¹⁹F (D₂O, 282 MHz) δ ppm −126.22. ES-MS 325 (M−1).

### Preparation of 3-[(2-fluorobenzyl)amino]-1-propanesulfonic acid

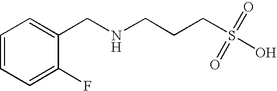

To a solution of 2-Fluorobenzylamine (5.0 g, 40.0 mmol) in 25% Toluene/Acetonitrile (40 mL) was added 1,3-propane sultone solution (4.65 g, 38.1 mmol). The solution was stirred at reflux for 4 hours. The reaction mixture was cooled to room temperature. The solid material was collected by filtration and washed with acetone (2×20 mL). The solid was suspended in EtOH (40 mL). The suspension was stirred at reflux for 1 hour. The mixture was cooled to room temperature, the solid material was collected by filtration, washed with acetone (2×20 mL) and dried in a vacuum oven (50° C.), affording the title compound, 7.76 g (82%). ¹H NMR (D₂O, 300 MHz) δ ppm 7.33 (m, 2H), 7.09 (m, 2H), 4.17 (m, 2H), 3.10 (t, 2H, J=7.9 Hz), 2.83 (t, 2H, J=7.3 Hz), 1.99 (m, 2H). ¹³C (D₂O, 75 MHz) δ ppm 162.65, 159.38, 132.40, 132.28, 132.18, 132.14, 125.33, 117.87, 117.67, 116, 13, 115.84, 48.21, 46.26, 45.13, 45.07, 21.65. ¹⁹F (D₂O, 282 MHz) δ ppm −117.66. ES-MS 246 (M−1).

### Preparation of 3-[(5-fluoro-2-methylbenzyl)amino]-1-propanesulfonic acid

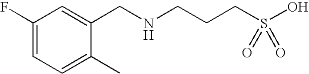

A mixture of 1,3-propane sultone (0.90 g, 7.3 mmol), 5-fluoro-2-methylbenzylamine (1.00 g, 7.2 mmol), acetonitrile (7.5 mL) and toluene (7.5 mL) was heated to reflux for 2 hours then was cooled to room temperature. The solid was collected by suction filtration, rinsed with acetone (2×5 mL). The solid was dried 18 hours at 60° C. in the vacuum oven. The title compound was obtained as a white solid (1.53 g, 5.85 mmol, 81%). ¹H NMR (500 MHz, DMSO-d6) δ 2.01 (qt, J=6.5 Hz, 2H), 2.33 (s, 3H), 2.68 (t, J=6.3 Hz, 2H), 3.18 (t, J=6.3 Hz, 2H), 4.14 (s, 2H), 7.14-7.18 (m, 1H), 7.28-7.32 (m, 2H), 9.001 (br s, 2H); ¹³C NMR (75 MHz, DMSO-d6) δ 18.1, 21.7, 47.1, 47.4, 49.3, 115.3 (d, J=20.7 Hz), 116.4 (d, J=23.0 Hz), 131.9 (d, J=8.1 Hz), 132.4 (d, J=6.9 Hz), 133.1 (d, J=2.3 Hz), 159.8 (d, J=241 Hz); ¹⁹F NMR (280 MHz, DMSO-d6) δ −117.391 (dd, J=15.4 and 8.8 Hz, 1F); ES-MS 260 (M−H).

### Preparation of 3-({1,1-dimethyl-2-[2-(trifluoromethoxy)phenyl]ethyl}amino)-1-propanesulfonic acid

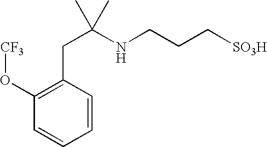

NaOMe (0.5M, 27.2 mL) was added to 2-nitropropane (1.2 g, 13.6 mmol) and the solution was stirred for 30 min then concentrated to afford a white solid. To this solid was added 2-trifluromethoxybenzylpyrridinium (3.88 g, 6.8 mmol) and DMSO (15 mL). The mixture was heated at 100° C. for 15 hours then cooled to room temperature and diluted with HCl (1M) and EtOAc. After separation of the two phases, the organic layer was washed twice with HCl (1M) then concentrated to obtain an oily crude, mixed with some solid. Methanol was added to precipitate the pyridinium byproduct which was filtered off, and the filtrate was concentrated and purified by column using Hex:EtOAc 90:10 to obtain the desired nitro but still contaminated with the pyridinium salt.

To a stirred solution of the nitro (600 mg) in methanol (20 mL) was added a spatula of Raney-Ni in water. The suspension was hydrogenated under atmospheric pressure of hydrogen for 15 hours (TLC indicates complete consumption of the starting material) then filtered on celite and concentrated under reduced pressure. The crude was purified by column using CH₂Cl₂:MeOH 80:10 to afford 400 mg of the corresponding amine.

To a stirred solution of the amine (400 mg, 1.7 mmol) in THF/pinacolone (2 mL/2 mL) was added 1,3-propane sultone (230 mg, 1.89 mmol). The reaction mixture was stirred at reflux for 15 hours then cooled to room temperature. The solid was collected by filtration and was washed with THF. The solid was suspended in EtOH (10 mL) and stirred at reflux for 1 hour. The suspension was then cooled to room temperature. The solid was collected by filtration, washed with ethanol and dried under high vacuum to afford the title compound, 590 mg (97%). ¹H NMR (500 MHz, DMSO-d₆) δ 1.15 (s, 6H), 2.00 (m, 2H), 2.69 (m, 2H), 2.99 (s, 2H), 3.15 (m, 2H), 7.40 (m, 4H), 8.80 (bs, 2H). ¹⁹F (282 MHz, DMSO-d₆) δ −56.46 (d, J=2.5 Hz, 3F). ¹³C NMR (125 MHz, DMSO-d₆) δ 23.24, 23.47, 37.62, 41.42, 49.84, 60.01, 120.83, 127.76, 128.05, 130.04, 134.29, 148.01. ES-MS 354 (M−1).

### Preparation of 3-{[1-(4-fluorophenyl)propyl]amino}-1-propanesulfonic acid

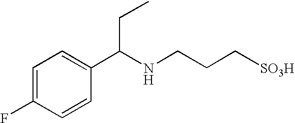

To a stirred solution of 4-fluorobenzaldehyde (1.24 g, 10 mmol) in THF (15 mL) at 0° C. was added dropwise LHMDS (2.0 g, 12 mmol) and the resulting solution was stirred for 20 min. EtMgBr was added dropwise and the mixture was refluxed for 24 hours. The mixture was cooled to room temperature, poured into saturated NH₄Cl (aq) and then extracted with EtOAc. The organic layers were combined and concentrated under reduced pressure. The crude residue was stirred with 3N HCl (25 mL) for 30 minutes and the aqueous layer extracted with EtOAc while the organic layer was discarded. The aqueous layer was cooled to 0° C. and treated with solid NaOH pellets until pH˜10 was attained. The aqueous layer was extracted with EtOAc and the organic layer was concentrated. The crude was purified by column using CH₂Cl₂/MeOH 95:05 to afford 700 mg of the desired amine (45% yield).

To a stirred solution of the amine (612 mg, 4.0 mmol) in THF (6 mL) was added 1,3-propane sultone (490 mg, 4.0 mmol). The reaction mixture was stirred at reflux for 15 hours then cooled to room temperature. The solid was collected by filtration and was washed with THF. The solid was suspended in EtOH (10 mL) and stirred at reflux for 1 hour. The suspension was then cooled to room temperature. The solid was collected by filtration, washed with ethanol and dried under high vacuum to afford the title compound, 800 mg (73% yield). ¹H NMR (300 MHz, D₂O) δ 0.59 (t, J=7.0 Hz, 3H), 1.80-2.01 (m, 4H), 2.72 (t, J=7.0 Hz, 2H), 2.75 (m, 1H), 2.95 (m, 1H), 4.00 (dd, J=10.0 and 7.0 Hz, 1H), 7.09 (m, 2H), 7.30 (m, 2H). ¹³NMR (125 MHz, D₂O) 89.82, 21.71, 26.01, 44.62, 48.21, 63.87, 116.19, 116.47, 129.62, 130.35, 130.45, 161.41, 164.66. ¹⁹F (282 MHz, D₂O) δ −112.82 (ht, J=5.0 Hz, 1F). ES-MS 274 (M−1).

### Preparation of 3-{[1-(4-fluorophenyl)propyl]amino}-1-propanesulfonic acid

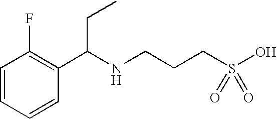

To a stirred solution of 2-fluorobenzaldehyde (1.24 g, 10 mmol) in THF (15 mL) at 0° C. was added dropwise LHMDS (2.0 g, 12 mmol) and the resulting solution was stirred for 20 min. EtMgBr was added dropwise and the mixture was refluxed for 24 hours. The mixture was cooled to room temperature, poured into saturated NH₄Cl (aq) and then extracted with EtOAc. The organic layers were combined and concentrated under reduced pressure. The crude residue was stirred with 3N HCl (25 mL) for 30 min and the aqueous layer extracted with EtOAc while the organic layer was discarded. The aqueous layer was cooled to 0° C. and treated with solid NaOH pellets until pH˜10 was attained. The aqueous layer was extracted with EtOAc and the organic layer was concentrated. The crude was purified by column using CH₂Cl₂/MeOH 95:05 to afford 800 mg of the desired amine (52% yield).

To a stirred solution of the amine (612 mg, 4.0 mmol) in THF (6 mL) was added 1,3-propane sultone (490 mg, 4.0 mmol). The reaction mixture was stirred at reflux for 15 hours then cooled to room temperature. The solid was collected by filtration and was washed with THF. The solid was suspended in EtOH (10 mL) and stirred at reflux for 1 hour. The suspension was then cooled to room temperature. The solid was collected by filtration, washed with ethanol and dried under high vacuum to afford the title compound, 430 mg (39% yield). ¹H NMR (300 MHz, D₂O) δ 0.65 (t, J=7.0 Hz, 3H), 1.82-2.01 (m, 4H), 2.76 (t, J=7.0 Hz, 2H), 2.86 (m, 1H), 3.00 (m, 1H), 4.36 (dd, J=10.0 and 5.0 Hz, 1H), 7.10 (m, 1H), 7.19 (m, 1H), 7.31-7.40 (m, 2H). ¹³NMR (125 MHz, D₂O) δ 9.46, 21.44, 25.00, 44.82, 48.10, 58.53, 116.33, 116.52, 125.55, 129.55, 132.09. ¹⁹F (282 MHz, D₂O) δ −118.12 (qt, J=5.0 Hz, 1F). ES-MS 274 (M−1).

### Preparation of 3-{[1-(3-fluorophenyl)propyl]amino}-1-propanesulfonic acid

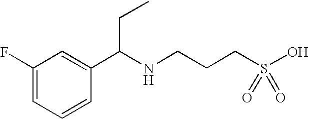

To a stirred solution of 2-fluorobenzaldehyde (1.24 g, 10 mmol) in THF (15 mL) at 0° C. was added dropwise LHMDS (2.0 g, 12 mmol) and the resulting solution was stirred for 20 min. EtMgBr was added dropwise and the mixture was refluxed for 6 hours. The mixture was cooled to room temperature, poured into satNH4Cl (aq) and then extracted with EtOAc. The organic layers were combined and concentrated under reduced pressure. The crude residue was stirred with 3N HCl (25 mL) for 30 minutes and the aqueous layer extracted with EtOAc while the organic layer was discarded. The aqueous layer was cooled to 0° C. and treated with solid NaOH pellets until pH˜10 was attained. The aqueous layer was extracted with EtOAc and the organic layer was concentrated. The crude was purified by column using CH₂Cl₂/MeOH 95:05 to afford 320 mg of the desired amine (21% yield).

To a stirred solution of the amine (306 mg, 2.0 mmol) in THF (4 mL) was added 1,3-propane sultone (270 mg, 2.2 mmol). The reaction mixture was stirred at reflux for 15 hours then cooled to room temperature. The solid was collected by filtration and was washed with THF. The solid was suspended in EtOH (10 mL) and stirred at reflux for 1 hour. The suspension was then cooled to room temperature. The solid was collected by filtration, washed with ethanol and dried under high vacuum to afford the title compound, 350 mg (63% yield). ¹H NMR (300 MHz, DMSO-d₆) δ 0.65 (t, J=7.0 Hz, 3H), 1.75-2.01 (m, 4H), 2.60 (t, J=7.0 Hz, 2H), 2.75 (m, 1H), 2.95 (m, 1H), 4.20 (m, 1H), 7.10-7.60 (m, 4H), 9.00-9.40 (bd, 2H). ¹³NMR (125 MHz, DMSO-d₆) δ 13.03, 24.96, 29.01, 48.30, 52.19, 65.09, 117.66, 117.99, 118.66, 118.93, 127.34, 133.95, 140.63. ¹⁹F (282 MHz, DMSO-d₆) δ −110.25 (m, 1F). ES-MS 274 (M−1).

### Preparation of 3-[2-(4-fluorophenyl)-2-hydroxy-1,1-dimethylethyl)amino]-1-propanesulfonic acid

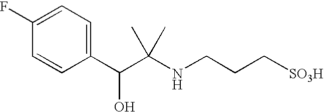

14 g of washed Amberlyst A21 ion exchange resin were placed in a round bottom flask to which was added nitropropane (14 mL, 120 mmol) and 4F-fluorobenzaldehyde (7.45 g, 60 mmol). The reaction mixture was stirred overnight then diluted with Et₂O and filtered. The filtrate was concentrated under rotavap vaccuo then pump vaccuo by heating at 120° C. to remove the excess of aldehyde. The crude was purified by column using Hex:EA 90:10 to afford 3.3 g (25%) of the Henry-aldol product as a colorless solid.

To a solution of the nitro compound (5 g, 23.5 mmol) in MeOH (100 mL) was added 6M HCl (25 mL). After cooling to 5° C., zinc powder (7.6 g, 117 mmol) was added. The suspension was stirred at room temperature for 3 hours then filtered on a celite pad. The filter cake was washed with MeOH (2×20 mL). The combined filtrates were evaporated under reduced pressure. The residue was dissolved in EtOAc (40 mL), then K₂CO₃ (1M) was added until basic pH. The organic phase was dried with Na₂SO₄, filtered, evaporated and dried in vacuo to afford 3.5 g (83% yield) of the corresponding amine. The amine was used without further purification.

To a stirred solution of the amine (3.3 g, 18.0 mmol) in THF (20 mL) was added 1,3-propane sultone (2.2 g, 18.0 mmol). The reaction mixture was stirred at reflux for 15 hours then cooled to room temperature. The solid was collected by filtration, washed with ethanol and with Et₂O then dried under high vacuum to afford the title compound, 4.25 g (77% yield). ¹H NMR (500 MHz, DMSO-d₆) δ 1.13 (s, 6H), 2.00 (m, 2H), 2.66 (dd, J=7.0 & 7.0 Hz, 2H), 2.75 (s, 2H), 3.10 (dd, J=7.0 & 7.0 Hz, 2H), 6.72 (d, J=8.3 Hz, 2H), 7.00 (d, J=8.3 Hz, 2H), 8.60 (bs, 2H), 9.36 (s, 1H). ¹³NMR (125 MHz, DMSO-d₆) δ 23.1, 41.2, 43.2, 49.8, 59.4, 115.7, 125.8, 132.3, 157.1. ¹⁹F (282 MHz, DMSO-d₆) δ −115.15 (m, 1F). ES-MS 304 (M−1).

### Preparation of 3-[(4-fluoro-3-methylbenzyl)amino]-1-propanesulfonic acid

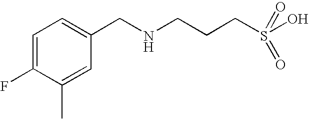

To a solution of 4-fluoro-3-methylbenzylamine (1.14 g, 8.2 mmol) in a solvent mixture of toluene and acetonitrile (12 mL, v/v=3/1) was added 1,3-propane sultone (0.953 g, 7.8 mmol). The solution was stirred at reflux for 3 hours. The reaction mixture was cooled to room temperature. The solid was collected by filtration and washed with acetone (2×10 mL). The solid was suspended in EtOH (15 mL). The suspension was stirred at reflux for 1 hour. The mixture was cooled to room temperature. The solid material was then collected by filtration, washed with acetone (2×10 mL) and dried in a vacuum oven (50° C.), affording the title compound (1.85 g, 91%). ¹H NMR (D₂O, 300 MHz) δ ppm 7.12 (m, 2H), 6.94 (t, 1H, J=9.1 Hz), 4.01 (s, 2H), 3.03 (t, 2H, J=7.3 Hz), 2.80 (t, 2H, J=7.3 Hz), 2.09 (s, 3H), 1.96 (m, 2H); ¹³C NMR (D₂O, 75 MHz) δ ppm 163.21, 159.98, 133.19, 133.12, 129.12, 128.99, 126.29, 115.84, 115.53, 50.70, 48.18, 45.86, 21.65, 13.99; ¹⁹F NMR (D₂O, 282 MHz) δ ppm −117.24; ES-MS 262 (M+1).

### Preparation of 3-{[3-fluoro-4-(trifluoromethyl)benzyl]amino}-1-propanesulfonic acid

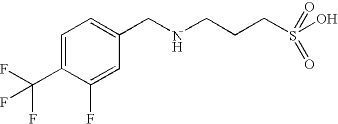

To a solution of 3-fluoro-4-(trifluoromethyl)benzylamine (1.38 g, 7.1 mmol) in a solvent mixture of toluene and acetonitrile (12 mL, v/v=1:3) was added 1,3-propane sultone (0.831 g, 6.8 mmol). The solution was stirred at reflux for 3 hours and then cooled to room temperature. The solid was collected by filtration and washed with acetone (2×10 mL). The solid was suspended in EtOH (15 mL) and stirred at reflux for 1 hour. After the mixture cooled to room temperature, the solid material was collected by filtration, washed with acetone (2×10 mL) and dried in a vacuum oven (50° C.), affording the title compound (1.54 g, 72%). ¹H NMR (D₂O, 300 MHz) δ ppm 7.62 (t, 1H, J=7.9 Hz), 7.27 (m, 2H), 4.16 (s, 2H), 3.10 (t, 2H, J=7.8 Hz), 2.83 (t, 2H, J=7.3 Hz), 1.99 (m, 2H); ¹³C NMR (D₂O, 75 MHz) δ ppm 161.05, 157.67, 137.51, 137.89, 128.35, 128.29, 125.76, 125.71, 124.22, 120.65, 118.91, 118.30, 118.02, 50.26, 48.13, 46.44, 21.65; ¹⁹F NMR (D₂O, 282 MHz) δ ppm −62.28, −114.76; ES-MS 316 (M+1).

### Preparation of 3-amino-2-(4-fluorobenzyl)-1-propanesulfonic acid

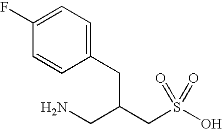

To a cold (−78° C.) solution of 3-hydroxypropionitrile (3.5 g, 50 mmol) in THF (100 mL), was added a solution of lithium bis(trimethylsilyl)amide (1M in THF, 100 mL). After stirring for 1 hour at this temperature, 4-fluorobenzyl bromide (6.13 mL, 50 mml) was added drop wise and the reaction was left warming to reach −10° C. over a period of 4 hours. The reaction was quenched with 1N HCl and extracted with EtOAc. The organic layer was washed with 1N HCl, dried over Na₂SO₄ and concentrated. The crude product was purified by column chromatography to afford 6 g (67%) of the desired monoalkylated product. The dialkylated product was isolated in 10% yield (1.5 g).

To a solution of 2-(4-fluorobenzyl)-3-hydroxypropionitrile (6 g, 33.48 mmol) in EtOH (100 mL) was added an aqueous solution of NH₄OH (30% in water, 40 mL) followed by Ra—Ni (3 g). The suspension was stirred under H₂ pressure of 50 psi for 5 hours. The catalyst was removed by filtration and the filtrate was concentrated under high vacuum for use in the next step.

To the crude 3-amino-2-fluorobenzyl-1-propanol was added HBr (48% in water, 75 mL) and the reaction mixture was heated under reflux for 15 hours. The reaction was diluted with H₂O to dissolve the solid product and the impurities were removed by filtration. The filtrate was concentrated to produce a white solid in almost quantitative yield.

A solution of the crude bromide in water (30 mL) was added dropwise to a refluxed solution of Na₂SO₃ (7.56 g, 60 mmol) in water (30 mL). After the end of the addition, the reaction mixture was stirred at reflux for 3 hours. The reaction mixture was cooled to room temperature and concentrated under reduced pressure. Concentrated HCl (70 mL) was added to dissolve the desired and precipitate the inorganic salts which were removed by filtration. The filtrate was concentrated to afford a white solid probably contaminated with salts (NaCl and NaBr) which were eliminated by adding H₂O (15 mL). The suspension was filtered to obtain 3.5 g of title compound as a white solid (47% yield). ¹H NMR (500 MHz, DMSO-d₆) δ 2.35 (m, 1H), 2.55-2.75 (m, 4H), 2.95 (m, 1H), 2.98 (m, 1H), 7.12 (m, 2H), 7.22 (m, 2H), 7.90 (bs, 2H). ¹³C NMR (125 MHz, DMSO-d₆) δ 36.55, 38.24, 54.53, 115.62, 115.90, 135.72, ¹⁹F (282 MHz, DMSO-d₆) δ −117.22 (m, 1F). ES-MS 246 (M−1).

### Preparation of 3-(N,N-dibenzylamino)-2,2-difluoropropane-1-sulfonic acid

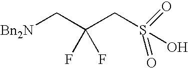

To a stirred solution of benzotriazol (6 g, 50 mmol) in MeOH (25 mL) was added dibenzylamine (10.57 mL, 55 mmol) and formaldehyde (37% in water, 4.9 mL). Two layers were formed after a few minutes. The mixture was made homogenous by adding Et₂O then the reaction mixture was heated at reflux overnight. After cooling, the reaction was diluted with H₂O (100 mL) then washed with brine. White solid was formed during the wash with brine. The aqueous solution was removed and the white solid was obtained by filtration. The product was washed with Et₂O and then dried under high vacuum to obtain 15 g (91% yield) of the desired product.

To a stirred suspension of Zinc dust (1.84 g, 27.6 mmol) in THF (40 mL) was added TMSCl (1.81 mL, 14.18 mmol) followed by the addition of methyl bromodifluoroacetate (3.16 g, 9.14 mmol). The mixture was stirred for 15 minutes, then a solution of benzotriazol reagent (obtained in step 1, 4.6 g, 14.16 mmol) in THF (20 mL) was added slowly. The reaction mixture was stirred for 2 hours. It was then diluted with aqueous solution of K₂CO₃ (1M, 25 mL) and EtOAC. The mixture was stirred vigorously. The organic layer was isolated and the aqueous layer was extracted with EtOAc. The combined organic layers were dried over Na₂SO₄ and concentrated. The pure product was obtained using column chromatography, yield 2.1 g (69%). ¹H NMR (500 MHz, CDCl₃) δ1.03 (t, J=7.0 Hz, 3H), 2.99 (t, J_{H-F}=11.5 Hz, 2H), 3.51 (s, 4H), 4.00 (q, J=7.0 Hz, 2H), 7.06-7.19 (m, 10H).

To a cold (−50° C.) solution of the methyl 3-(N,N-dibezylamino)-2,2-difluoropropionate (1 g, 3 mmol) in THF (60 mL) was added in four portions LiAlH₄ (228 mg, 6 mmol). The cooling bath was removed and the reaction was stirred at room temperature for 1 hour. The reaction was quenched by the addition of NaOH (1M) and diluted with Et₂O. The mixture was vigorously stirred for 1 hour before the two phases were separated. The organic layer washed with brine then dried (Na₂SO₄) and concentrated. The crude was applied to flash column chromatography (eluent: Hex:EtOAc 80:20) to afford 700 mg (80%) of the desired alcohol.

To a stirred solution of 3-(N,N-dibenzylamino)-2,2-difluoropropanol (1 g, 3.43 mmol) in CH₂Cl₂ (30 mL) was added NEt₃ (580 μL, 4.12 mmol) followed by MsCl (193 mL, 3.77 mmol). The reaction mixture was stirred for 2 hours, then H₂O was added and the reaction mixture was extracted with EtOAc. After evaporation of the organic layers the crude product obtained was used in the next step.

The crude mesylate (obtained in step 4) was dissolved in EtOH (15 mL) and was added slowly to a refluxed solution of Na₂SO₃ (760 mg, 6 mmol) in H₂O (15 mL). The reaction was stirred at reflux for 4 hours, then an additional 300 mg of Na₂SO₃ was added and the reaction was stirred for 2 more hours at reflux and overnight at room temperature. The solvent was evaporated and the mixture was diluted in a minimum of water (10 ml) to dissolve the salt. After filtration, the obtained white solid was suspended in EtOH and heated at reflux with stirring for 30 minutes. After cooling, the product was obtained as a white solid (1 g, yield 94%) after filtration. ¹H NMR (500 MHz, D₂O with a drop of NaOH (40% in D₂O)) δ2.71 (t, J_{H-F}=14.0 Hz, 2H), 3.20 (t, J_{H-F}=15.0 Hz, 2H), 3.58 (s, 4H), 7.19-7.30 (m, 10H). ¹⁹F NMR (282 MHz, D₂O with a drop of NaOH (40% in D₂O)) δ −110.38 (quintuplet, J=15.0 Hz, CF₂), ES-MS 354 (M−1).

### Preparation of 3-amino-2,2-difluoropropane-1-sulfonic acid

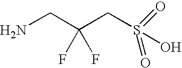

Pd(OH)₂/C (50 mg) was added to a solution of 3-(N,N-dibenzylamino)-2,2-difluoropropane-1-sulfonic acid (100 mg, 0.28 mmol) in EtOH/H₂O/AcOH (30 mL/30 mL/10 mL). The suspension was stirred overnight under atmospheric pressure of H₂. The suspension was filtered and the filtrate was concentrated to afford a white solid which was suspended in EtOH (5 mL). After stirring the suspension for 10 minutes, the product was collected by filtration to obtain 48 mg (98%) of a colorless solid. ¹H NMR (500 MHz, D₂O) δ3.60 (t, J_{F-H}=15.0 Hz, 2H). 3.62 (t, J=16.0 Hz, 2H). ¹³C NMR (125 MHz, D₂O) δ 43.01 (t, J_{F-C}=25.0 Hz), 53.96 (t, J_{F-C}=25.0 Hz) 117.86 (t, J=245 Hz). ¹⁹F NMR (282 MHz, D₂O) δ −102.04 (quintuplet, J_{H-F}=15.0 Hz, CF₂). ES+MS 176 (M+1).

### Preparation of 3-amino-3-(4-fluorophenyl)-1-propanesulfonic acid

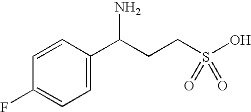

A solution of borane:tetrahydrofurane complex (1M, 100 mL) was added dropwise over 1 hour to a cold (0° C.) suspension of DL-3-amino-3-(4-fluorophenyl)propionoc acid (7.30 g, 39.9 mmol) in THF (40 mL). The mixture was heated to reflux for 22 hours. The mixture was then cooled to 0° C. and methanol (35 mL) was added over 15 minutes. The mixture was then heated to reflux for 30 minutes and concentrated to a thick oil. The oil was coevaporated 3 times with methanol (50 mL). The crude product was used directly in next step.

The oil that was obtained in the previous step was dissolved in water and added dropwise to concentrated HBr (44 mL) The solution was heated at reflux for 18 hours. It was then concentrated to dryness (11.58 g). The solid was suspended in hot heptane/2-butanone then cooled to room temperature. Ether was added and the mixture was stirred for 30 minutes. The solid was collected by filtration and rinsed with ether (9.97 g, about 66% for two steps).

The 3-bromo-1-(4-fluorophenyl)-1-propylamine hydrobromide (obtained in step 2, 32 mmol) was added to a solution of sodium sulfite (3.78 g, 30 mmol) in water (40 mL). The mixture was heated at 90° C. for 2.5 hours, and was then concentrated to a thick paste. Concentrated HCl (8 mL) was added to the paste. The resulting suspension was stirred for 20 minutes at room temperature. The solid was collected by filtration and rinsed with concentrated HCl (3×30 mL). The filtrated solid was concentrated to dryness. The solid was washed in ethanol/toluene then dried in vacuo (3.79 g). The solid was recrystallized in ethanol (25 mL) and water (6 mL). After cooling to room temperature, the solid was collected by filtration, rinsed with ethanol (2×5 mL) and dried overnight at 60° C. in a vacuum oven. The title compounds was obtained as a fine white crystalline solid, yield 2.37 g, 26% overall yield. ¹H NMR ¹H (500 MHz, D₂O) δ 2.22-2.36 (m, 2H), 2.54-2.60 (m, 1H), 2.65-2.71(m, 1H), 4.37-4.40 (m, 1H), 7.07 (t, J=8.5 Hz, 2H), 7.26-7.31 (dd, J=8.3, 5.4 Hz, 2H); ¹³C (125 MHz, D₂O) δ 28.82, 47.1, 53.5, 116.4 (d, J=22 Hz, 2C), 139.7 (d, J=11.6 Hz, 2C), 130.9, 163.2 (d, J=246 Hz, 2C); ¹⁹F (282 MHz, D₂O) −112.9 to −113.0 (m); ES-MS 232 (M−1).

### Preparation of 3-[2-(4-fluorophenyl)-2-propylamino]-1-propanesulfonic acid

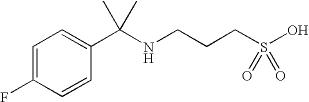

A mixture of 1-(4-fluorophenyl)-1-methylethylamine (5.09 mmol, 0.78 g), 1,3-propane sultone (5.10 mmol, 0.65 g), MeCN (7 mL) and toluene (3 mL) was heated under reflux for 6 h. After cooling to 5° C. (ice/water bath), tert-butyl methylether was added. The solid was collected by filtration, rinsed with tert-butyl methylether (3×4 mL) and dried overnight at 60° C. in a vacuum oven (1.20 g). The solid obtained was suspended in ethanol (7 mL) and the mixture was heated to reflux for 1 hour. After cooling to room temperature, the solid was collected by filtration, rinsed with ethanol (2×2 mL) and dried for 2 hours at 60° C. in the vacuum oven. The desired material was obtained as a white solid, yield 1.16 g, 83%. ¹H NMR (500 MHz, DMSO-d6) δ 1.65 (s, 6H), 1.91 (qt, J=6.3 Hz, 2H), 2.60 (t, J=6.3 Hz, 2H), 2.5 (br s, 2H), 7.30-7.33 (m, 2H), 7.60-7.63 (m, 2H), 9.21 (br s, 2H); ¹³C (125 MHz, DMSO-d6) δ 22.05, 25.22, 41.89, 49.46, 59.60, 115.63 (d, J=21.1 Hz), 128.56 (d, J=8.6 Hz), 135.83, 161.91 (d, J=245 Hz); ¹⁹F (282 MHz, DMSO-d6) −114.0 to −114.1 (m); ES-MS 274 (M−1).

### Preparation of 3-[(5-fluoro-2,3-dihydro-1H-inden-1-yl)amino]propane-1-sulfonic acid

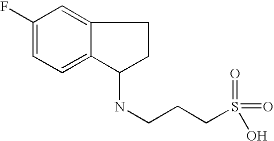

To a stirred solution of 5-fluoro-1-indanone (1 g, 6.7 mmol) in ethanol (20 ml) was added a suspension of hydroxylamine hydrochloride (1.11 g, 16 mmol, 2.4 eq.) in ethanol/water (2 mL/2 mL), followed by the addition of a suspension of sodium acetate (1.31 g, 16 mmol, 2.4 eq.) in ethanol/water (2 mL/2 mL). The reaction mixture was heated to reflux for 3.5 hours. Water was added and the white solid was collected by filtration (1.1 g, 99%).

Palladium on activated carbon 10% (200 mg) was added to a solution flushed with nitrogen of the 5-fluoroindan-1-one oxime obtained in Step 1 (1.1 g, 6.6 mmol) in methanol (90 mL) and acetic acid (10 mL). The reaction mixture was flushed with H₂ and left under H₂ atmosphere overnight. The reaction mixture was flushed with nitrogen and filtered through Celite. The filtrate was concentrated and azeotroped twice with toluene to yield the desired amine (100%).

To a solution of the amine (6.6 mmol, from Step 2) in a 25% acetonitrile/toluene solution (50 mL) was added 1,3-propanesultone (766 mg, 6.3 mmol, 0.95 eq.). The reaction mixture was stirred at reflux for 4 hours. The white solid obtained was collected by filtration, put into ethanol and reflux for 1 hour. After cooling the solid obtained was collected by filtration and dried under vacuum, to give a white solid product (1.16 g, 68%): ¹H NMR (DMSO, 500 MHz) δ ppm 9.16 (bs, 1H), 8.96 (bs, 1H), 7.60 (dd, 1H, J=8.3 and 5.4 Hz), 7.21 (dd, 1H, J=2 and 9 Hz), 7.17-7.13 (m, 1H), 4.73-4.70 (m, 1H), 3.18-3.13 (m, 2H), 3.11-3.06 (m, 1H), 2.92-2.86 (m, 1H), 2.71-2.65 (m, 2H), 2.47-2.41 (m, 1H), 2.21-2.14 (m, 1H), 2.03-1.96 (m, 2H); MS 272 (M−1).

### Preparation of 3-[2-(4-trifluoromethylphenyl)-2-propylamino]-1-propanesulfonic acid

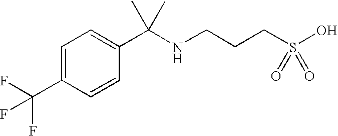

A mixture of 1-(4-trifluoromethylphenyl)-1-methylethylamine (8.30 mmol, 1.69 g), 1,3-propane sultone (8.5 mmol, 0.75 mL), MeCN (7 mL) and toluene (3 mL) was heated to reflux for 24 hours. After cooling to room temperature, the solid was collected by filtration, rinsed with ethanol (3×5 mL) and dried for 1 hour at 60° C. in a vacuum oven (2.47 g). The solid obtained was suspended in 95% ethanol (20 mL) and the mixture was heated to reflux for 1 hour. After cooling to room temperature, the solid was collected by filtration, rinsed with ethanol (2×5 mL) and dried for 2 hours at 60° C. in a vacuum oven. The desired material was obtained as a white solid (2.39 g, 89%). ¹H NMR (300 MHz, DMSO-d6) δ 1.69 (s, 6H), 1.94 (qt, J=6.4 Hz, 2H), 2.60 (t, J=6.4 Hz, 2H), 2.79 (br s, 2H), (q, J=8.5 Hz, 4H), 9.32 (br s, 2H); ¹³C (75 MHz, DMSO-d6) δ 22.25, 25.15, 41.97, 49.26, 59.93, 123.70 (q, J=271 Hz), 125.52 (d, J=3.5 Hz), 126.89, 128.71 (q, J=31.9 Hz)143.84; ¹⁹F (282 MHz, DMSO-d6) −61.87 (s); ES-MS 324 (M−1).

### Preparation of 4-{[(1S)-1-(4-fluorophenyl)ethyl]amino}-2-butanesulfonic acid

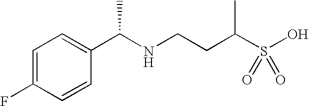

To a solution of (S)-(−)-1-(4-fluorophenyl)ethylamine (2.89 g, 20.7 mmol) in cosolvent of toluene and acetonitrile (20 mL, v/v=1:3) was added 2,4-butanesultone (2.69 g, 19.8 mmol). The solution was stirred under reflux for 4 hours. The reaction mixture was cooled to room temperature. The solid was collected by filtration and was washed with acetone (2×20 mL). The solid was suspended in EtOH (30 mL). The suspension was stirred at reflux for 1 hour. The mixture was cooled to room temperature. The solid was collected by filtration, washed with acetone (2×20 mL) and dried in a vacuum oven (50° C.), affording the title compound, 3.20 g (59%). ¹H NMR (D₂O, 500 MHz) δ ppm 7.34 (dd, 2H, J=5.4 Hz, 8.8 Hz), 7.08 (t, 2H, J=8.8 Hz), 4.29 (q, 1H, J=6.8 Hz), 2.96 (m, 1H), 2.97 (m, 1H), 2.79 (m, 2H), 1.97 (m, 1H), 1.73 (m, 1H), 1.52 (d, 3H, J=7.3 Hz), 1.08 (m, 3H); ¹³C (D₂O, 125 MHz) δ ppm 164.19, 162.23, 131.68, 129.91, 129.84, 116.43, 116.26, 57.84, 57.79, 53.20, 43.42, 28.14, 28.07, 18.26, 18.17, 14.67, 14.61. [α]_{D}=−19.9° (c=0.0100 in water), ES-MS 274 (M−1).

### Preparation of 4-{[(1S)-1-(4-fluorophenyl)ethyl]amino}-1-phenyl-2-butanesulfonic acid

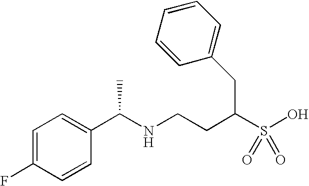

To a −78° C. solution of 1,3-propane sultone (5.0 g, 41 mmol) in anhydrous THF (150 mL) was added butyl lithium (2.5 M in hexanes, 18 mL, 41 mmol). The solution was stirred at −78° C. for 0.5 hour before benzyl bromide (4.9 mL, 41 mmol) was added via syringe pump over 0.5 hour period. The reaction mixture was stirred at −78° C. for 2 hours. The reaction mixture was warmed up to 0° C. before water (100 mL) was slowly added. The organic layer was recovered. The aqueous phase was extracted EtOAc (2×25 mL). The organic extracts were combined, dried over Na₂SO₄, filtered and evaporated under reduced pressure. The product was purified by flash chromatography (Rf=0.14, 80% Hex/EtOAc) affording the corresponding 1-benzyl-1,3-propanesultone (5.23 g, 60%).

To a solution of (S)-(−)-1-(4-fluorophenyl)ethylamine (1.0 g, 7.2 mmol) in cosolvent of toluene and acetonitrile (10 mL, v/v=1:3) was added a solution of 1-benzyl-1,3-propanesultone (1.45 g, 6.8 mmol). The solution was stirred at reflux for 4 hours. The reaction mixture was stirred overnight at room temperature. The solid was collected by filtration, washed with acetone (2×20 mL) and dried in a vacuum oven (50° C.), affording the title compound, 1.20 g (50%). ¹H NMR (500 MHz, D₂O) δ(ppm) 1.39 (m, 3H), 1.65 (m, 0.5H), 1.80 (m, 0.5H), 1.95 (m, 0.5H), 2.15 (m, 0.5H), 2.46 (m, 2H), 2.67 (m, 1H), 2.93 (m, 1H), 3.26 (m, 1H), 4.02 (m, 0.5H), 4.09 (m, 0.5H), 7.12 (m, 9H); ¹³C NMR (125 MHz, D₂O) δ(ppm) 18.26, 18.45, 25.51, 26.12, 35.82, 36.08, 43.26, 43.71, 57.20, 57.62, 59.24, 59.32, 116.26, 116.31, 116.44, 116.49, 127.17, 127.19, 129.01, 129.07, 129.10, 129.25, 129.67, 129.74, 129.81, 131.40, 137.73, 137.87, 162.17, 164.11; ¹⁹F NMR (D₂O, 282 MHz) −113.08; [α]_{D}=−20.1° (c=0.0026 in water); ES-MS 350 (M−1).

1. A compound of Formula I: 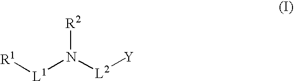
wherein:
R¹ is fluorine, hydrogen, a substituted or unsubstituted cycloalkyl, a substituted or unsubstituted aryl, a substituted or unsubstituted acyl, a substituted or unsubstituted arylcycloalkyl, a substituted or unsubstituted bicyclic or tricyclic ring, a bicyclic or tricyclic fused ring group, or a substituted or unsubstituted C₂-C₁₀ alkyl group;
R² is hydrogen, fluorine, a substituted or unsubstituted acyl, a substituted or unsubstituted alkyl, a substituted or unsubstituted mercaptoalkyl, a substituted or unsubstituted alkenyl, a substituted or unsubstituted alkynyl, a substituted or unsubstituted cycloalkyl, a substituted or unsubstituted aryl, a substituted or unsubstituted arylalkyl, a substituted or unsubstituted thiazolyl, a substituted or unsubstituted triazolyl, a substituted or unsubstituted imidazolyl, a substituted or unsubstituted benzothiazolyl, or a substituted or unsubstituted benzoimidazolyl;
Y is SO₃⁻X⁺, OSO₃⁻X⁺, SSO₃⁻X⁺, SO₂⁻X⁺, or CO₂⁻X⁺;
X⁺ is hydrogen or a cationic group; and
L¹ and L² are each independently a substituted or unsubstituted C₁-C₁₂ alkyl group or absent;
and pharmaceutically acceptable salts, esters, or prodrugs thereof, provided that at least one of R¹, R², L¹, or L² comprise one or more fluorine atoms, provided that when L² comprises one fluorine atom and Y is SO₂⁻X⁺, at least one of R¹ and R² is not hydrogen; and provided that when Y is CO₂⁻X⁺, and L² is C₂ substituted with an aryl group, then at least one of R¹ and R² are not hydrogen.

2.-43. (canceled)

44. The compound of formula (I), wherein said compound is selected from the group consisting of: 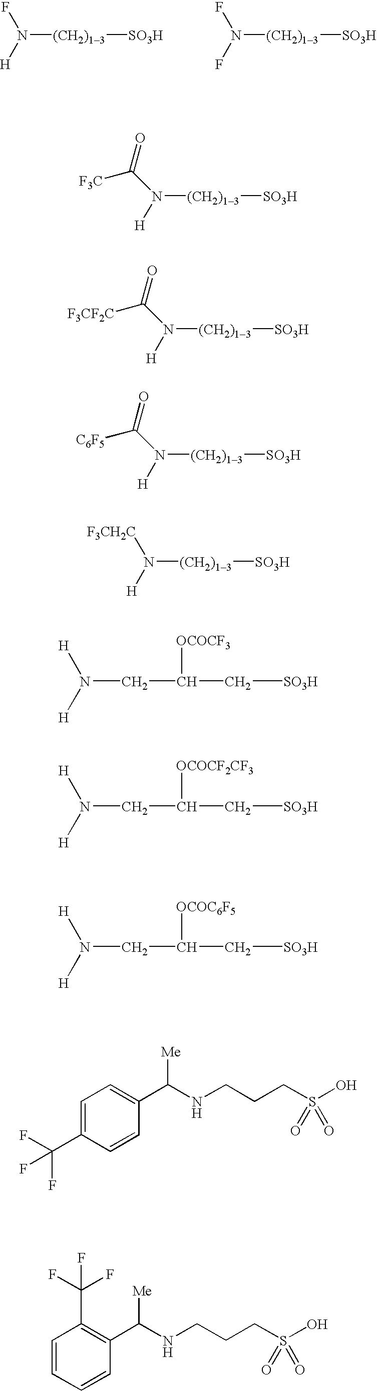 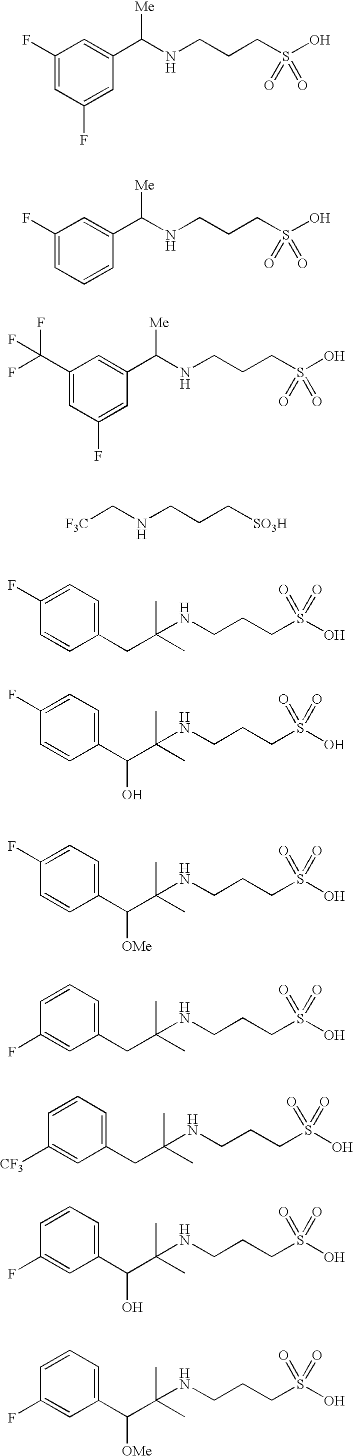 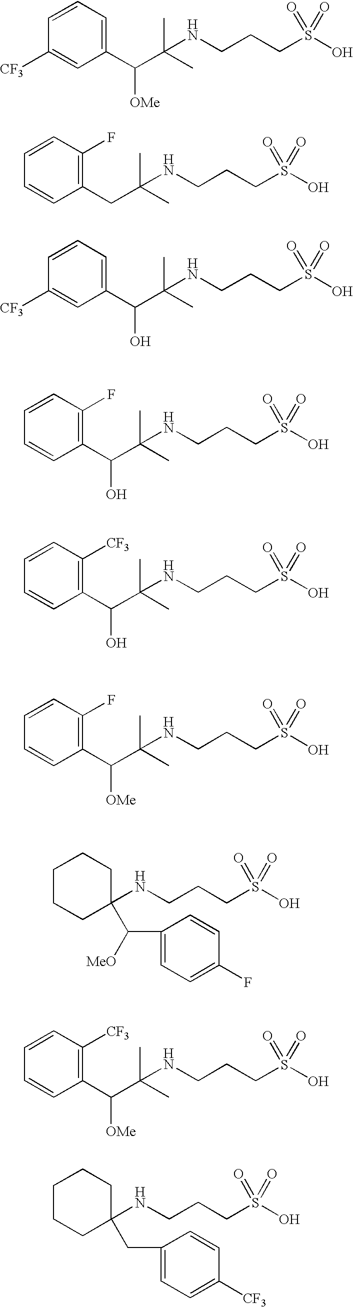 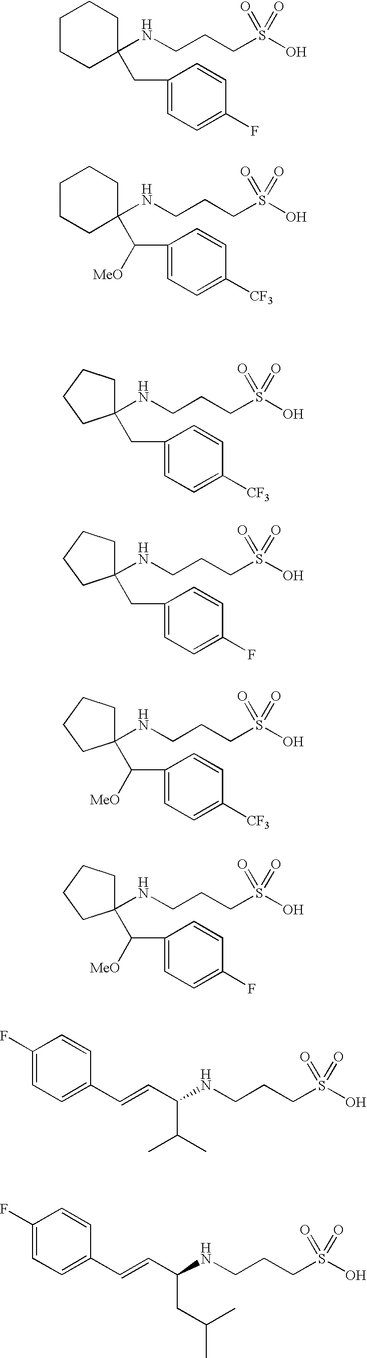 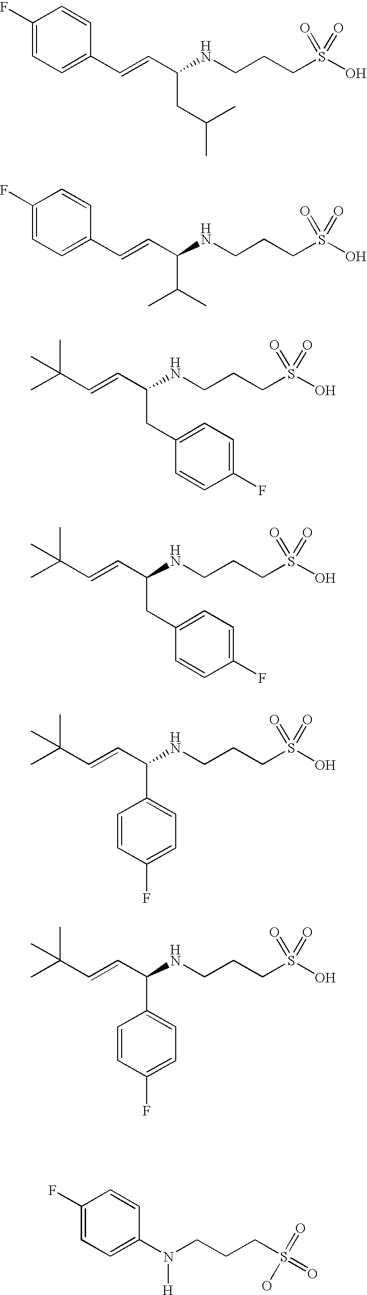 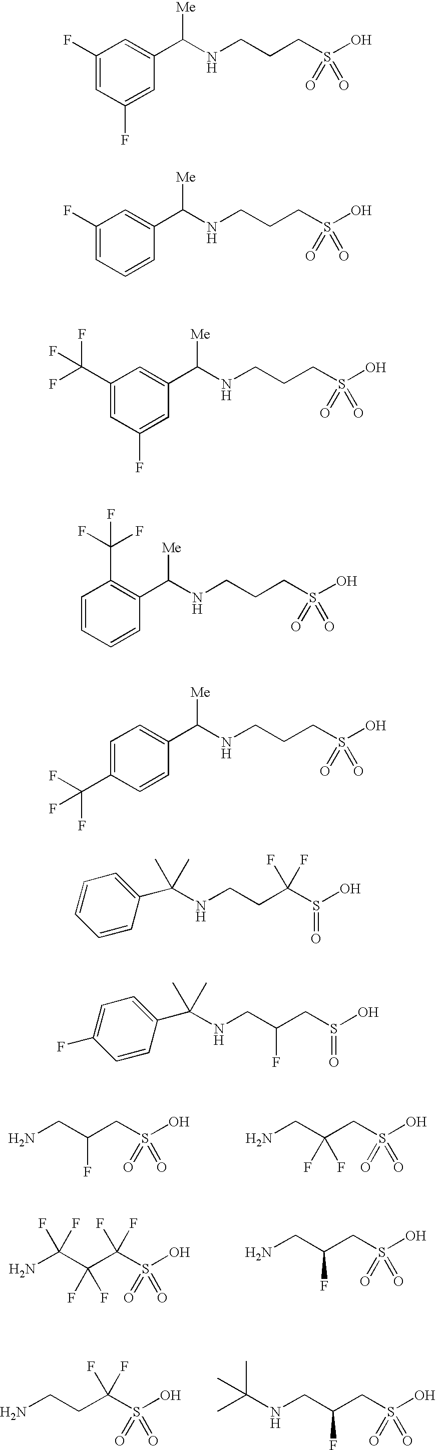 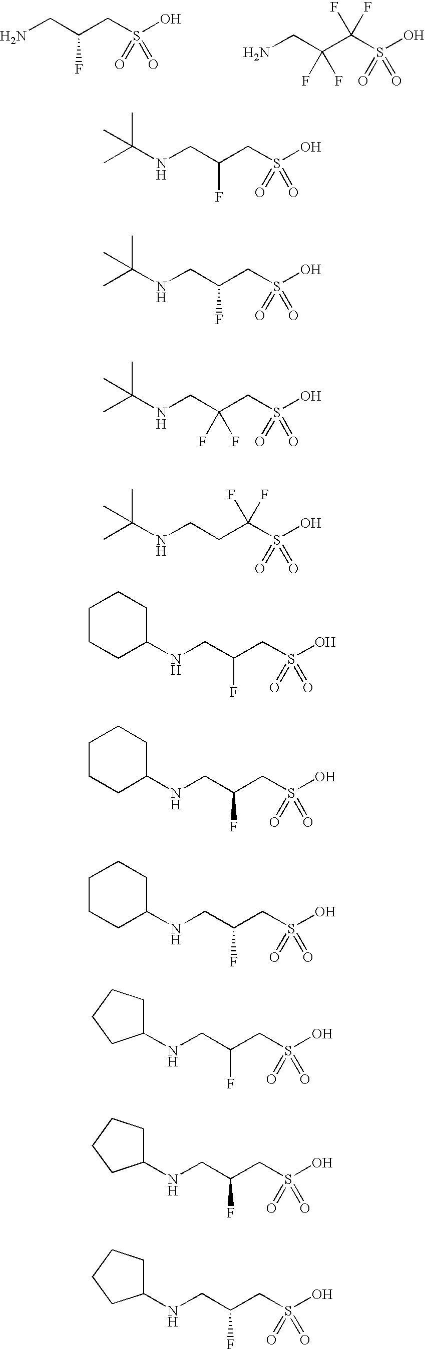 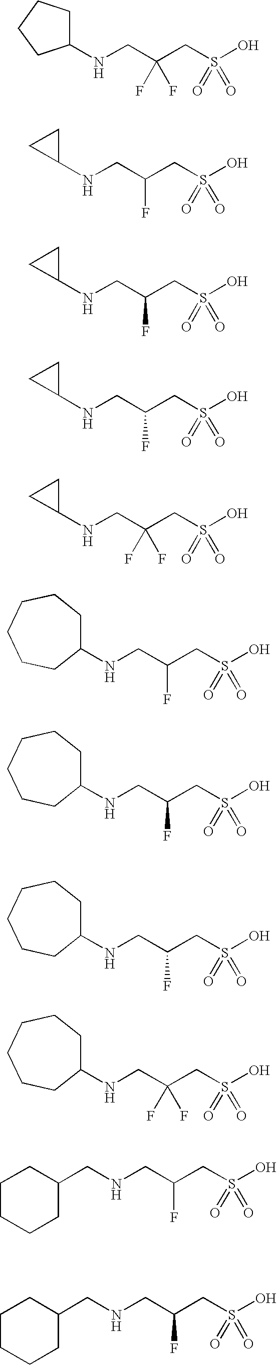 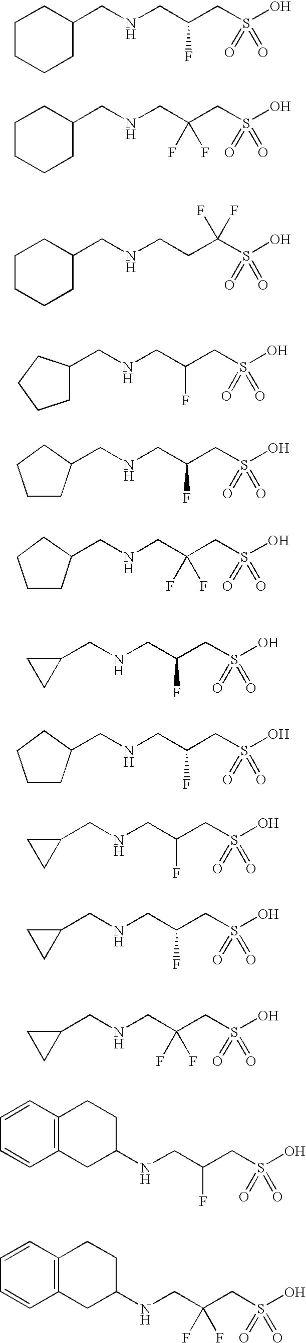 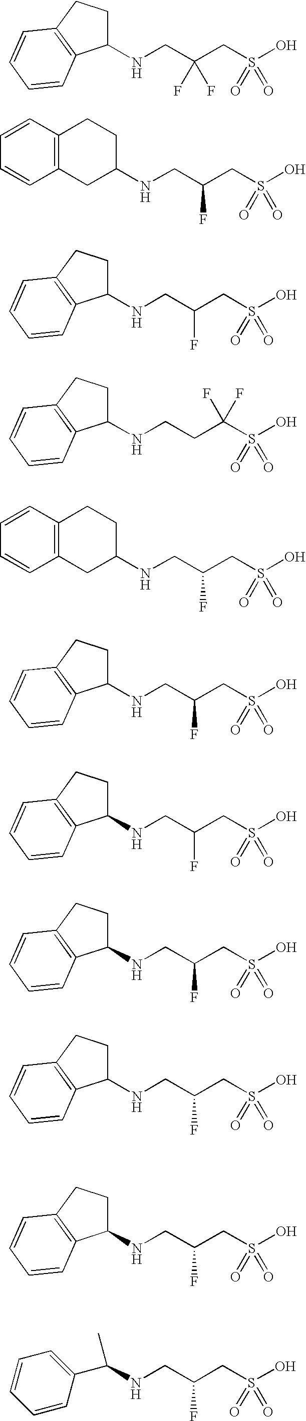 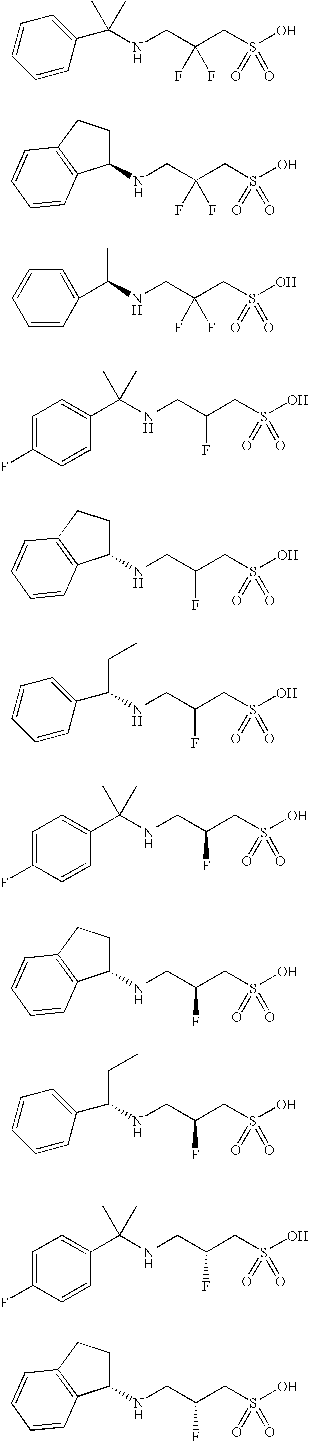 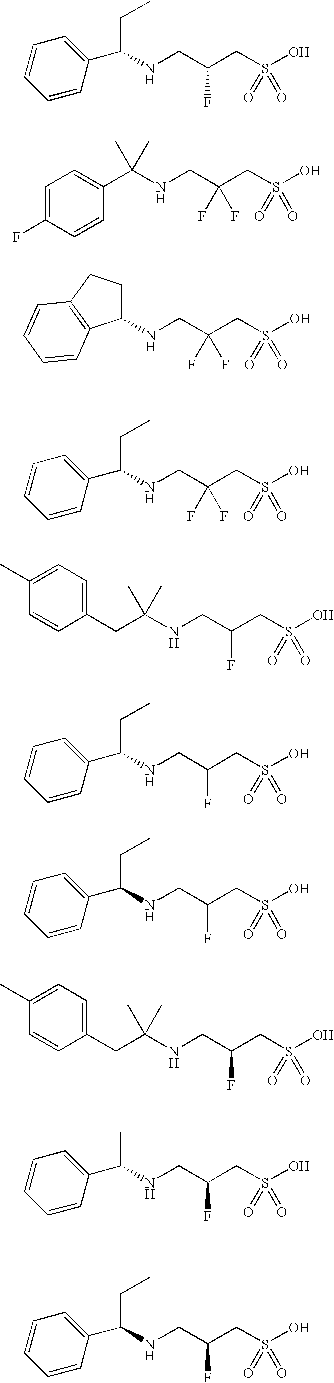 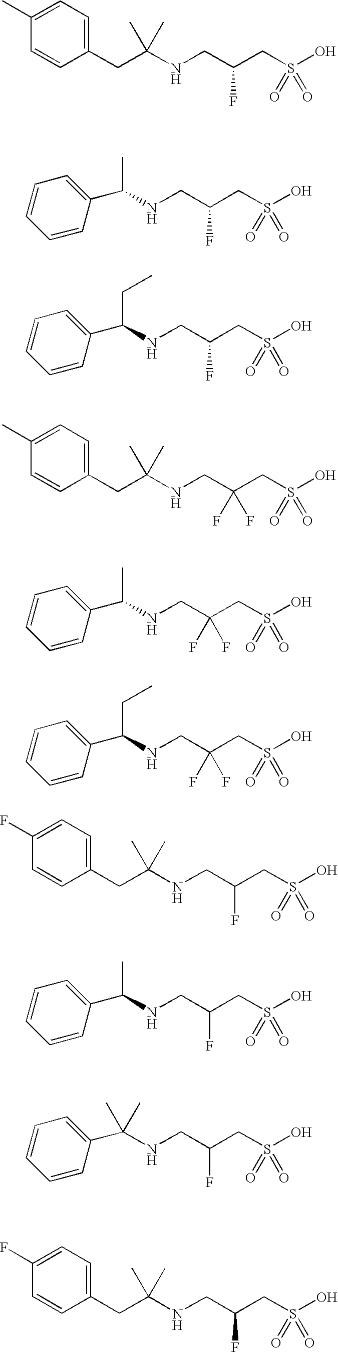 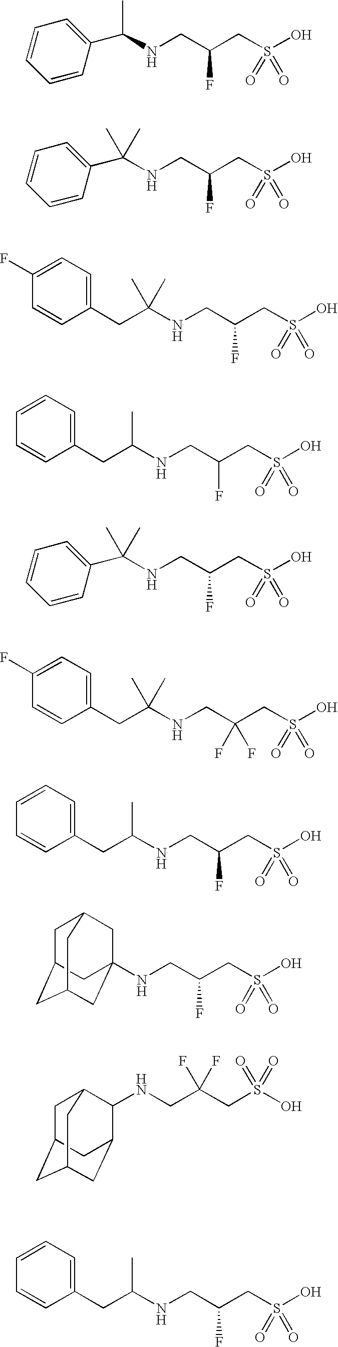 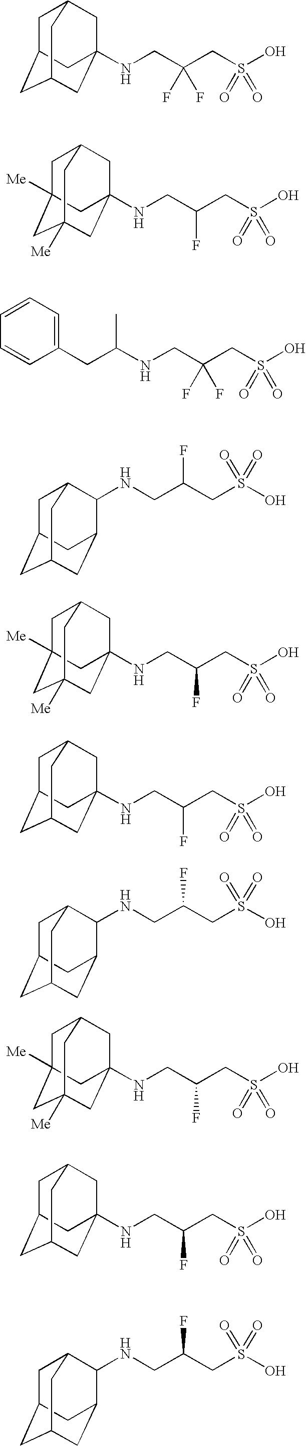 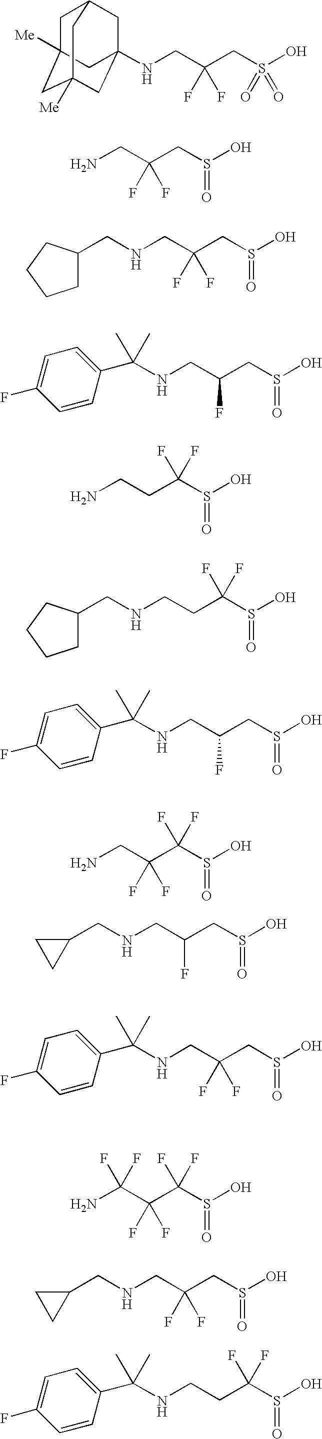 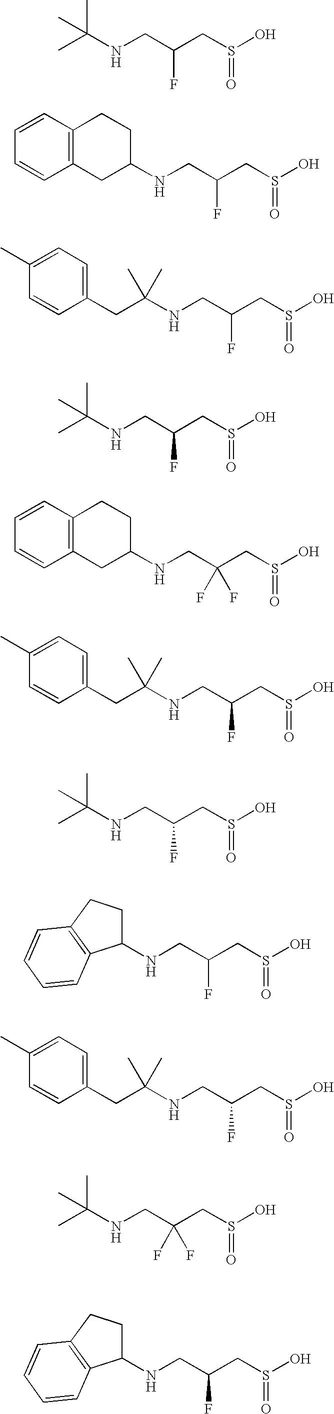 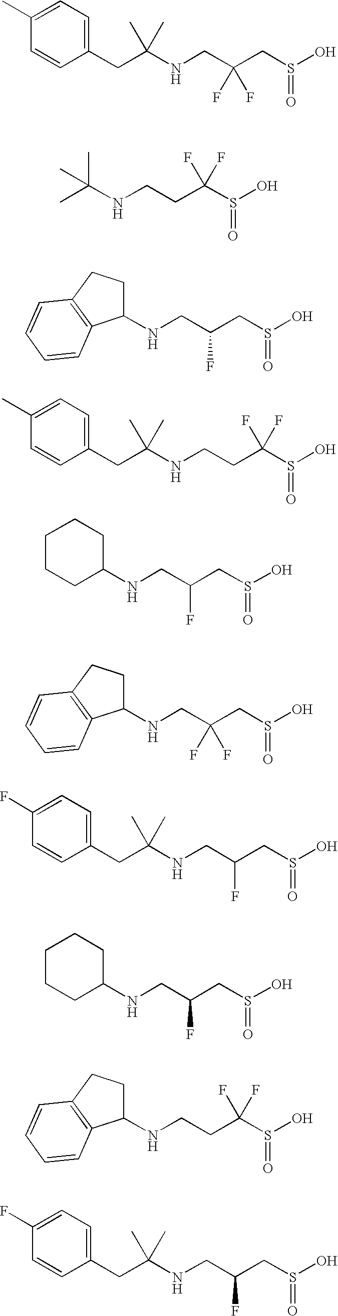 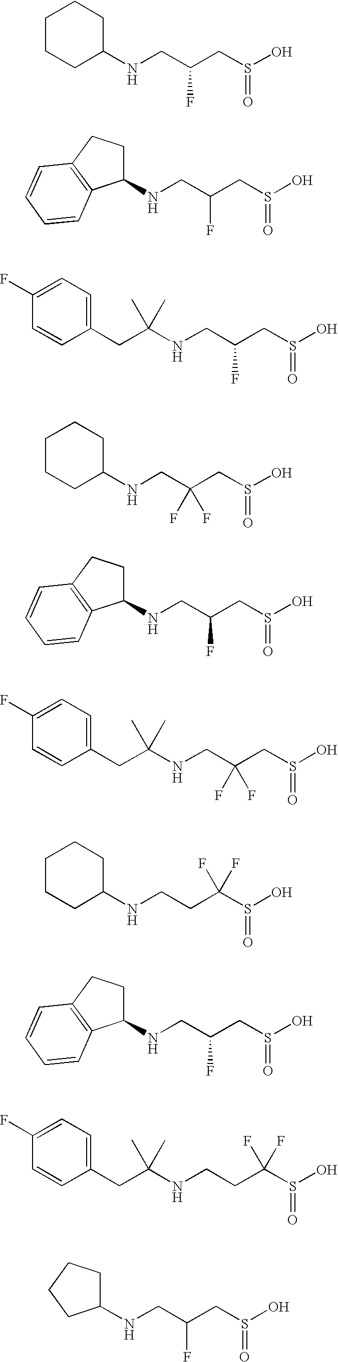 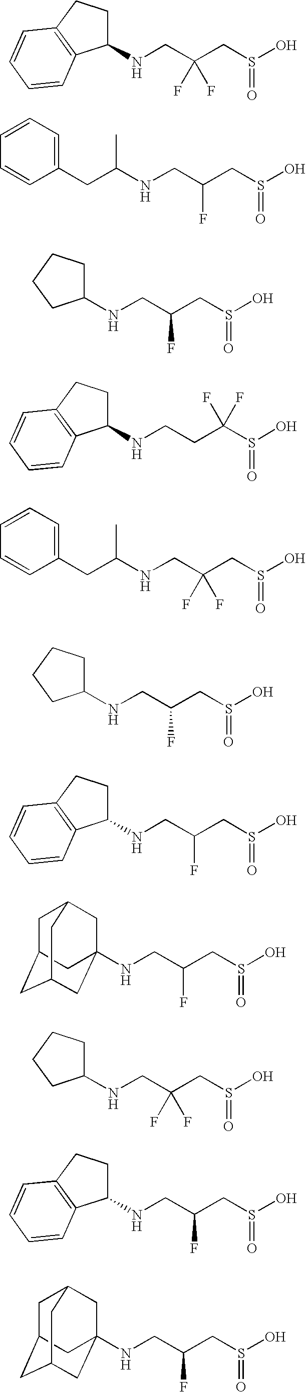 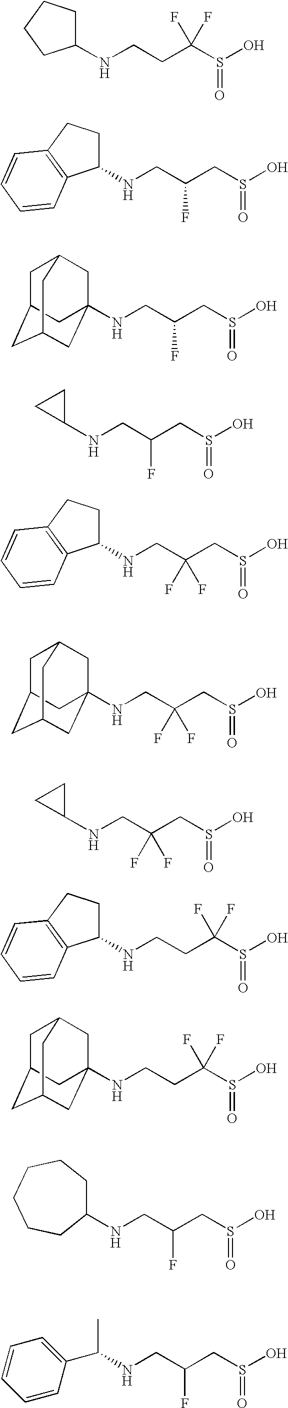 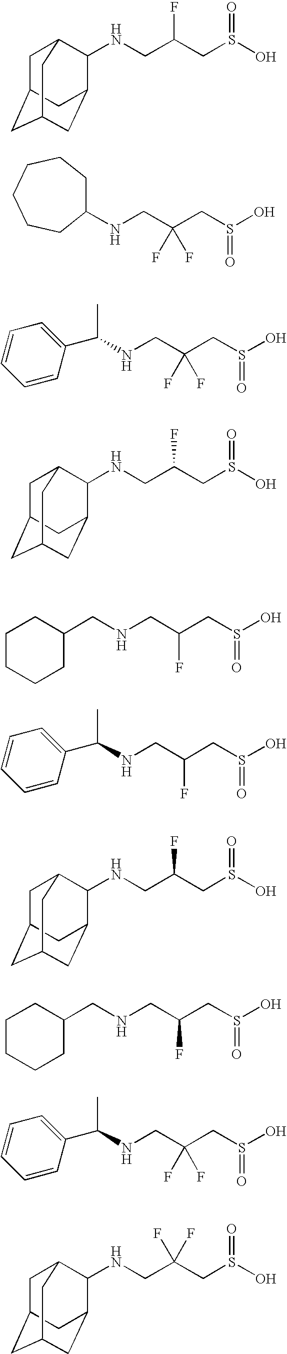 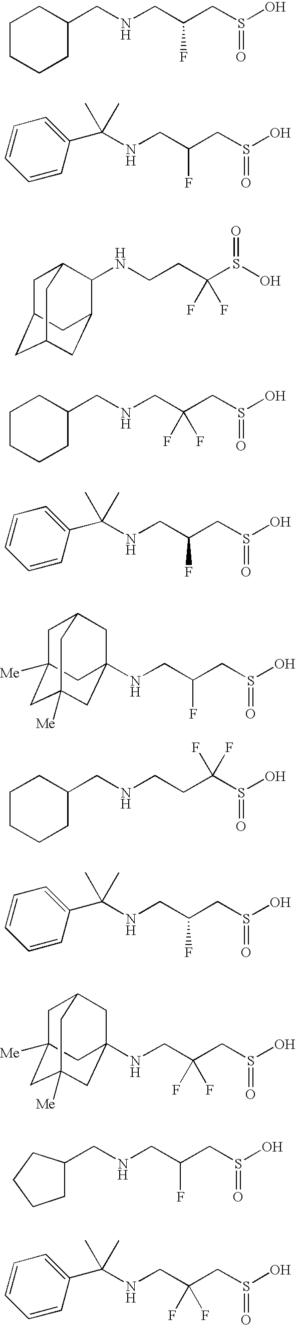 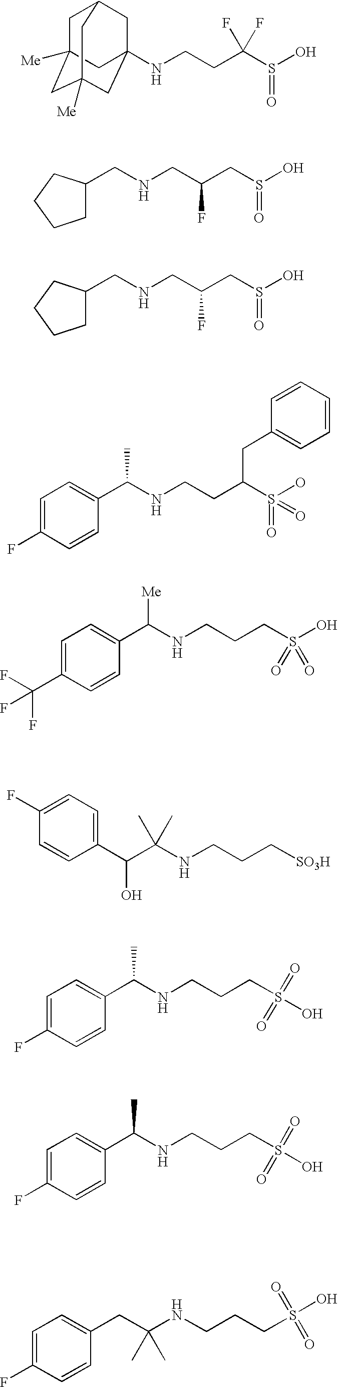 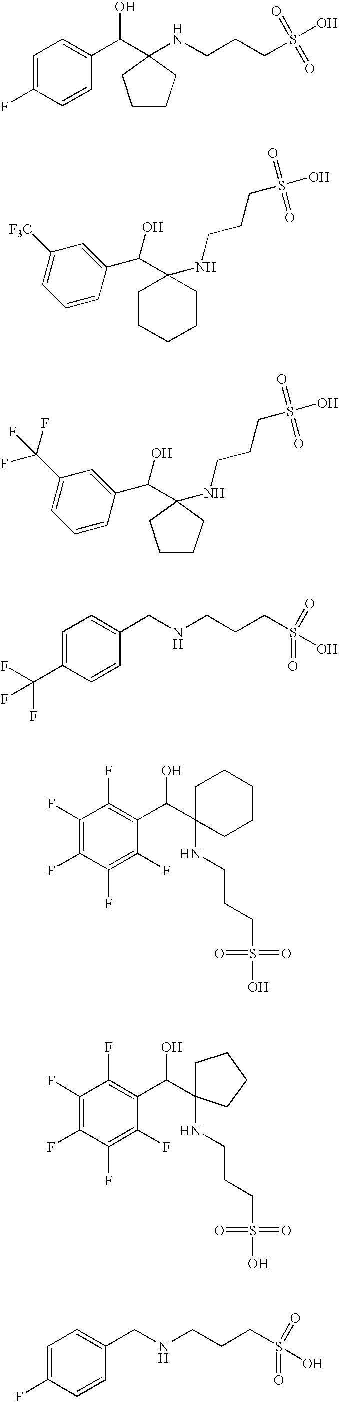 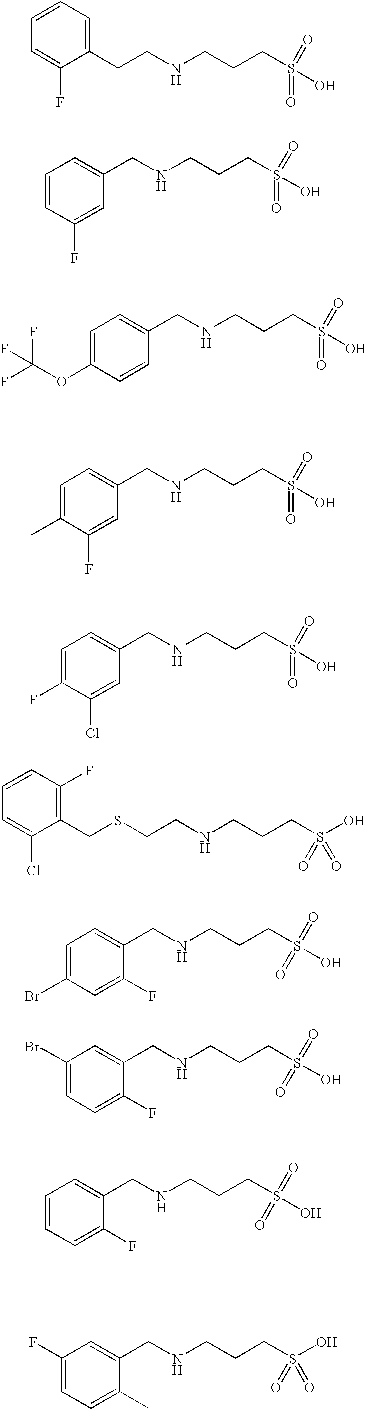 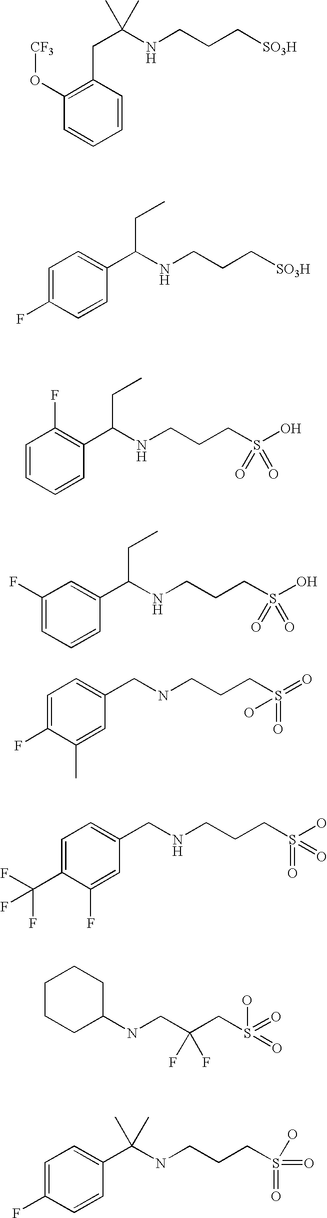 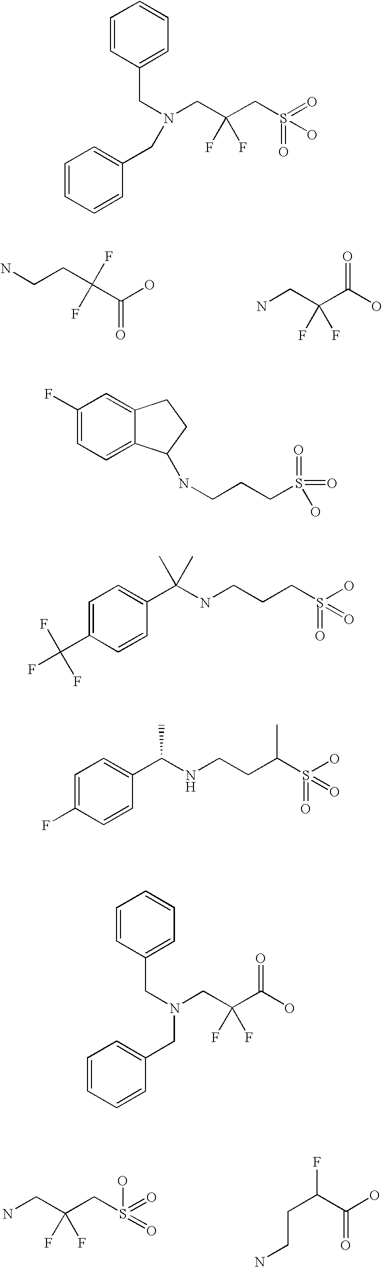
and pharmaceutically acceptable salts, esters, or prodrugs thereof.

45. A compound of formula (II): 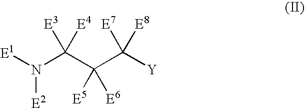
wherein:
E¹ and E² are each independently hydrogen or fluorine;
E³, E⁴, E⁵, E⁶, E⁷, and E⁸ are each independently is fluorine, hydrogen, a substituted or unsubstituted cycloalkyl, a substituted or unsubstituted aryl, a substituted or unsubstituted acyl, a substituted or unsubstituted arylcycloalkyl, a substituted or unsubstituted bicyclic or tricyclic ring, a bicyclic or tricyclic fused ring group, or a substituted or unsubstituted C₂-C₁₀ alkyl group;
Y is SO₃⁻X⁺, OSO₃⁻X⁺, SSO₃⁻X⁺, or SO₂⁻X⁺;
X⁺ is hydrogen or a cationic group; and pharmaceutically acceptable salts, esters, or prodrugs thereof, provided that at least one of E¹, E², E³, E⁴, E⁵, E⁶, E⁷, and E⁸ comprise one or more fluorine atoms.

46.-63. (canceled)

64. The compound of formula (II), wherein said compound is selected from the group consisting of: 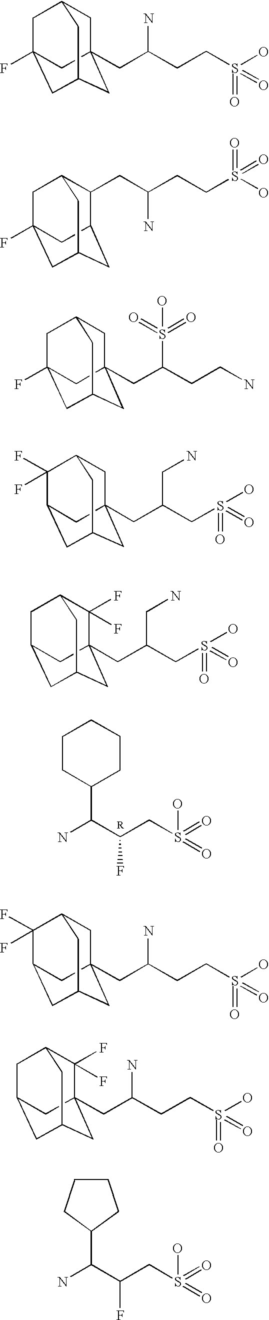 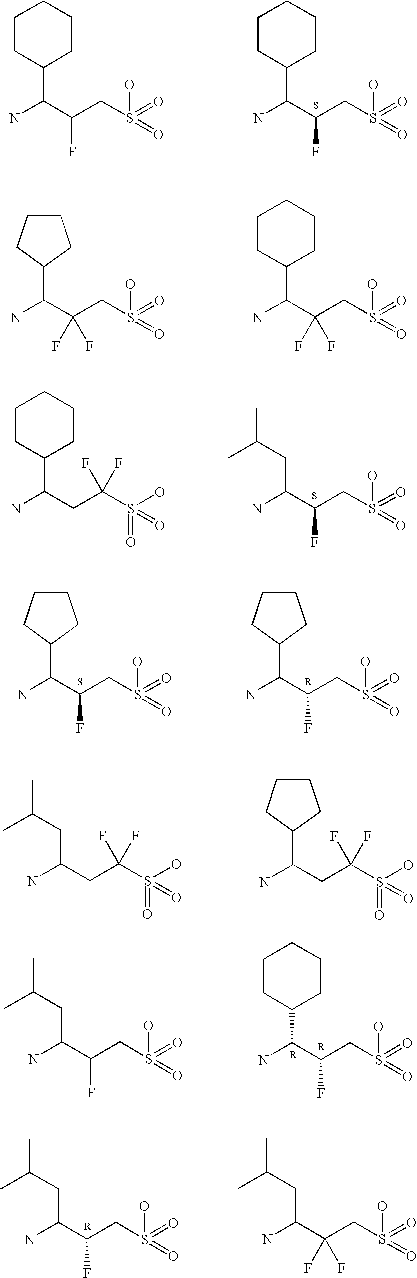 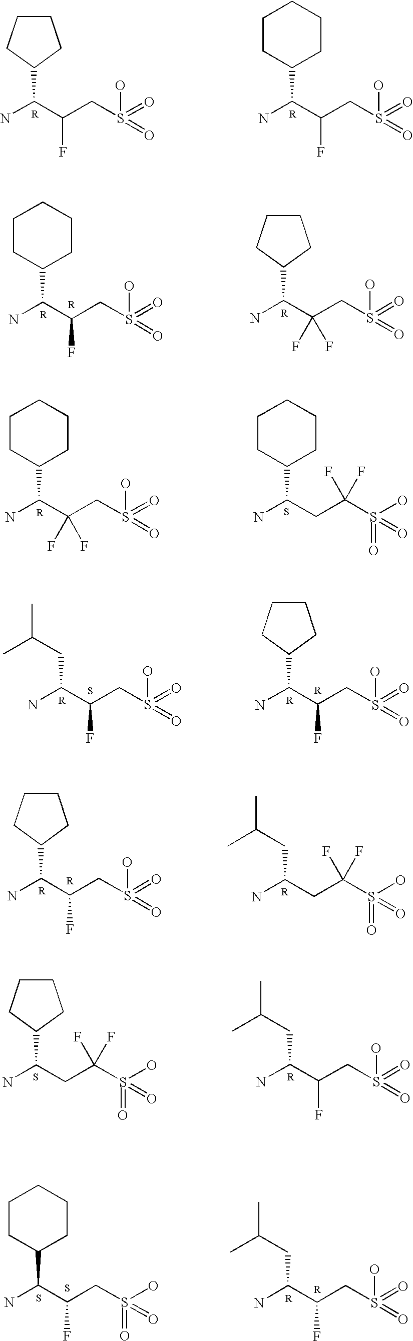 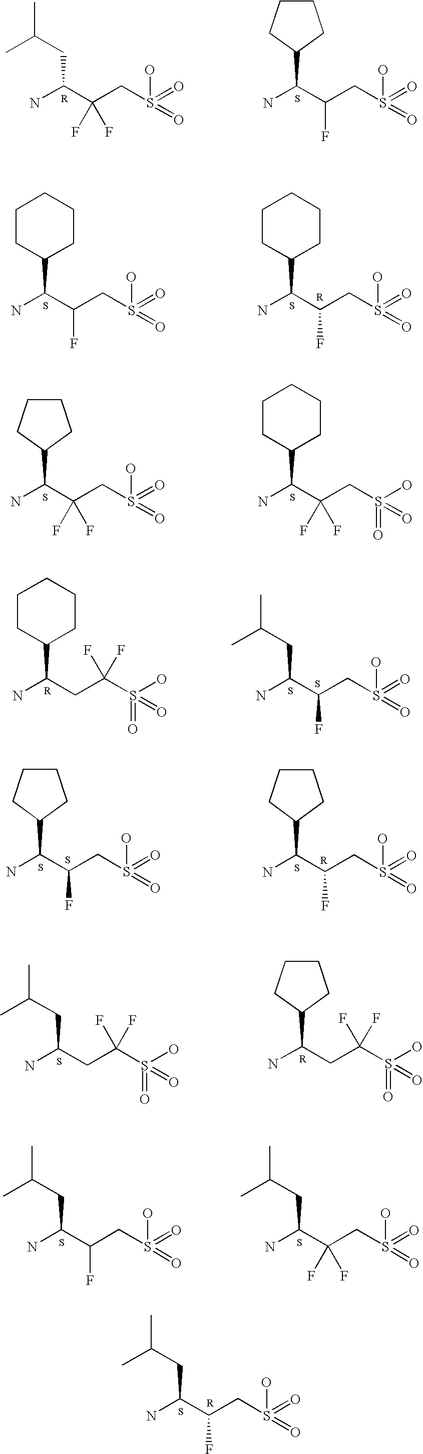 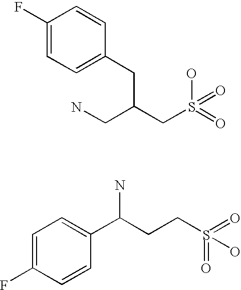
and pharmaceutically acceptable salts, esters, or prodrugs thereof.

65. A kit for use in treating amyloid related disease comprising a compound of claim 1, and instructions for use in the method of the instant invention.

66. A method of treating an amyloid-related disease in a subject, comprising administering to a subject in need thereof a compound of claim 1 or depicted in the Tables in an amount effective to treat an amyloid related disease.

67. The method according to claim 66, wherein said amyloid-related disease is Alzheimer's disease, cerebral amyloid angiopathy, inclusion body myositis, macular degeneration, MCI, or Down's syndrome.

68. The method of claim 66, according to claim 66, wherein amyloid fibril formation or deposition, neurodegeneration, microglial inflammatory response, cellular toxicity, or neuronal cell death is reduced or inhibited upon administration of said compound.

69. The method according to claim 66, wherein said amyloid-related disease is diabetes, AA amyloidosis, AL amyloidosis, or hemodialysis related amyloidosis (β₂M).

70. The method according to claim 66, wherein said subject is a human.

71. The method of claim 66, wherein said subject has Alzheimer's disease, Mild Cognitive Impairment, or cerebral amyloid angiopathy, and stabilization of cognitive function, prevention of a further decrease in cognitive function, or prevention, slowing, or stopping of disease progression occurs in said patient upon administration.

72. A pharmaceutical composition for the treatment or prevention of an amyloid-related disease comprising a compound of claim 1.

73. A pharmaceutical composition comprising a compound of claim 1.

74. A method of treating Alzheimer's Disease in a subject, comprising administering to a subject in need thereof a compound of claim 1 in an amount effective to treat Alzheimer's Disease.

75. A method of treating Mild Cognitive Impairment in a subject, comprising administering to a subject in need thereof a compound of claim 1 in an amount effective to treat Mild Cognitive Impairment.

76. A method of treating neurotoxicity associated with Aβ amyloid in a subject, comprising administering to a subject in need thereof a compound of claim 1 in an amount effective to treat neurotoxicity associated with Aβ amyloid.
